SEQUENCE LISTING (24 sequences)

SEQ ID NO: 1 - protein, 282 aa, synthetic construct
MGARASGSKSGSGSDSGSKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDLQSRPEPTAPPEESFRSGVETTTPPQKQEPIDKELYPLTSLRSLFGNDPSSQ

SEQ ID NO: 2 - protein, 411 aa, synthetic construct
MGARASGSKSGSGSDSGSKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDLQSRPEPTAPPEESFRSGVETTTPPQKQEPIDKELYPLTSLRSLFGNDPSSQASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 3 - protein, 411 aa, synthetic construct
MGARASGSKSGSGSDSGSKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYEQKLISEEDLQSRPEPTAPPEESFRSGVETTTPPQKQEPIDKELYPLTSLRSLFGNDPSSQ

SEQ ID NO: 4 - protein, 412 aa, synthetic construct
MGARASGSKSGSGSDSGSKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDLASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYLQSRPEPTAPPEESFRSGVETTTPPQKQEPIDKELYPLTSLRSLFGNDPSSQ

SEQ ID NO: 5 - protein, 278 aa, synthetic construct
MGCIKSKRKDNLNLQSRPEPTAPPEESFRSGVETTTPPQKQEPIDKELYPLTSLRSLFGNDPSSQKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDL

SEQ ID NO: 6 - protein, 407 aa, synthetic construct
MGCIKSKRKDNLNLQSRPEPTAPPEESFRSGVETTTPPQKQEPIDKELYPLTSLRSLFGNDPSSQKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDLASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 7 - protein, 407 aa, synthetic construct
MGCIKSKRKDNLNLQSRPEPTAPPEESFRSGVETTTPPQKQEPIDKELYPLTSLRSLFGNDPSSQKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYEQKLISEEDL

SEQ ID NO: 8 - protein, 407 aa, synthetic construct
MGCIKSKRKDNLNLQSRPEPTAPPEESFRSGVETTTPPQKQEPIDKELYPLTSLRSLFGNDPSSQASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDL

SEQ ID NO: 9 - protein, 396 aa, synthetic construct
MHGLQDDPDLQALLKGSQLLKVKSSSWRRERFYKLQEDCKTIWQESRKVMRSPESQLFSIEDIQEVRMGHRTEGLEKFARDIPEDRCFSIVFKDQRNTLDLIAPSPADAQHWVQGLRKIIHHSGSMDQRQKLQSRPEPTAPPEESFRSGVETTTPPQKQEPIDKELYPLTSLRSLFGNDPSSQKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDL

SEQ ID NO: 10 - protein, 525 aa, synthetic construct
MHGLQDDPDLQALLKGSQLLKVKSSSWRRERFYKLQEDCKTIWQESRKVMRSPESQLFSIEDIQEVRMGHRTEGLEKFARDIPEDRCFSIVFKDQRNTLDLIAPSPADAQHWVQGLRKIIHHSGSMDQRQKLQSRPEPTAPPEESFRSGVETTTPPQKQEPIDKELYPLTSLRSLFGNDPSSQKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDLASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 11 - protein, 525 aa, synthetic construct
MHGLQDDPDLQALLKGSQLLKVKSSSWRRERFYKLQEDCKTIWQESRKVMRSPESQLFSIEDIQEVRMGHRTEGLEKFARDIPEDRCFSIVFKDQRNTLDLIAPSPADAQHWVQGLRKIIHHSGSMDQRQKLQSRPEPTAPPEESFRSGVETTTPPQKQEPIDKELYPLTSLRSLFGNDPSSQKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYEQKLISEEDL

SEQ ID NO: 12 - protein, 525 aa, synthetic construct
MHGLQDDPDLQALLKGSQLLKVKSSSWRRERFYKLQEDCKTIWQESRKVMRSPESQLFSIEDIQEVRMGHRTEGLEKFARDIPEDRCFSIVFKDQRNTLDLIAPSPADAQHWVQGLRKIIHHSGSMDQRQKLQSRPEPTAPPEESFRSGVETTTPPQKQEPIDKELYPLTSLRSLFGNDPSSQASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDL

SEQ ID NO: 13 - protein, 500 aa, synthetic construct
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTERQANFLGKIWPSYKGRPGNFLQSRPEPTAPPEESFRSGVETTTPPQKQEPIDKELYPLTSLRSLFGNDPSSQ

SEQ ID NO: 14 - protein, 534 aa, synthetic construct
MGQTVTTPLSLTLGHWKDVERIAHNQSVDVKKRRWVTFCSAEWPTFNVGWPRDGTFNRDLITQVKIKVFSPGPHGHPDQVPYIVTWEALAFDPPPWVKPFVHPKPPPPLPPSAPSLPLEPPRSTPPRSSLYPALTPSLGAKPKPQVLSDSGGPLIDLLTEDPPPYRDPRPPPSDRDGNGGEATPAGEAPDPSPMASRLRGRREPPVADSTTSQAFPLRAGGNGQLQYWPFSSSDLYNWKNNNPSFSEDPGKLTALIESVLITHQPTWDDCQQLLGTLLTGEEKQRVLLEARKAVRGDDGRPTQLPNEVDAAFPLERPDWDYTTQAGRNHLVHYRQLLLAGLQNAGRSPTNLAKVKGITQGPNESPSAFLERLKEAYRRYTPYDPEDPGQETNVSMSFIWQSAPDIGRKLERLEDLKNKTLGDLVREAEKIFNKRETPEEREERIRRETEEKEERRRTEDEQKEKERDRRRHREMSKLLATVVSGQKQDRQGGERRRSQLDRDQCAYCKEKGHWAKDCPKKPRGPRGPRPQTSLL

SEQ ID NO: 15 - protein, 628 aa, synthetic construct
MGQTVTTPLSLTLGHWKDVERIAHNQSVDVKKRRWVTFCSAEWPTFNVGWPRDGTFNRDLITQVKIKVFSPGPHGHPDQVPYIVTWEALAFDPPPWVKPFVHPKPPPPLPPSAPSLPLEPPRSTPPRSSLYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTERQANFLGKIWPSYKGRPGNFLQSRPEPTAPPEESFRSGVETTTPPQKQEPIDKELYPLTSLRSLFGNDPSSQASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 16 - protein, 629 aa, synthetic construct
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTERQANFLGKIWPSYKGRPGNFLQSRPEPTAPPEESFRSGVETTTPPQKQEPIDKELYPLTSLRSLFGNDPSSQASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 17 - protein, 629 aa, synthetic construct
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTEASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYRQANFLGKIWPSYKGRPGNFLQSRPEPTAPPEESFRSGVETTTPPQKQEPIDKELYPLTSLRSLFGNDPSSQ

SEQ ID NO: 18 - protein, 616 aa, synthetic construct
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTHNPPIPVGEIYKRWIILGLNKIVRMYSPTSILDIRQGPKEPFRDYVDRFYKTLRAEQASQEVKNWMTETLLVQNANPDCKTILKALGPGATLEEMMTACQGVGGPGHKARVLAEAMSQVTNPATIMIQKGNFRNQRKTVKCFNCGKEGHIAKNCRAPRKKGVASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYTERQANFLGKIWPSHKGRPGNFLQSRPEPTAPPEESFRFGEETTTPSQKQEPIDKELYPLASLRSLFGSDPSSQ

SEQ ID NO: 19 - protein, 608 aa, synthetic construct
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMLQRMKQLEDKVEELLSKNYHLENEVARLKKLVGEFLGKIWPSYKGRPGNFLQSRPEPTAPPEESFRSGVETTTPPQKQEPIDKELYPLTSLRSLFGNDPSSQASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 20 - protein, 629 aa, synthetic construct; identical to SEQ ID NO: 16

SEQ ID NO: 21 - protein, 608 aa, synthetic construct
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMLQRMKQLEDKVEELLSKNYHLENEVARLKKLVGEASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYFLGKIWPSYKGRPGNFLQSRPEPTAPPEESFRSGVETTTPPQKQEPIDKELYPLTSLRSLFGNDPSSQ

SEQ ID NO: 22 - protein, 540 aa, synthetic construct
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMLQRMKQLEDKVEELLSKNYHLENEVARLKKLVGEASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 23 - protein, 322 aa, synthetic construct
MGARASVSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMLQRMKQLEDKVEELLSKNYHLENEVARLKKLVGELQSRPEPTAPPEESFRSGVETTTPPQKQEPIDKELYPLTSLRSLFGNDPSSQASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 24 - protein, 270 aa, synthetic construct
MGARASVSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMLQRMKQLEDKVEELLSKNYHLENEVARLKKLVGEASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

(19) United States
(12) Patent Application Publication
(10) Pub. No.: US 2023/0071834 A1
(43) Pub. Date: Mar. 9, 2023

(54) EXPORTED RNA REPORTERS FOR LIVE-CELL MEASUREMENT

(71) Applicant: California Institute of Technology, Pasadena, CA (US)

(72) Inventors: Richard F. Horns, Pasadena, CA (US); Michael B. Elowitz, Pasadena, CA (US)

(21) Appl. No.: 17/820,235

(22) Filed: Aug. 16, 2022

Related U.S. Application Data
(60) Provisional application No. 63/234,085, filed on Aug. 17, 2021.

(51) Int. Cl. C12N 15/10; C12N 15/11; C12N 15/62

(52) U.S. Cl. CPC C12N 15/1086; C12N 15/111; C12N 15/62; C12N 2320/10; C07K 2319/02; C07K 2319/03; C07K 2319/735; C07K 2319/85

(57) ABSTRACT

Disclosed herein include methods, compositions, and kits suitable for use in the measurement of the states of living cells across time. There are provided, in some embodiments, RNA exporter proteins comprising an RNA-binding domain, a membrane-binding domain, and an interaction domain capable of nucleating self-assembly. Disclosed herein include polynucleotides encoding reporter RNA molecule(s). In some embodiments, a plurality of RNA exporter proteins are capable of self-assembling into lipid-enveloped nanoparticles (LNs) secreted from a reporter cell in which the RNA exporter proteins are expressed, thereby generating a population of LNs comprising exported reporter RNA molecule(s).

### RELATED APPLICATIONS

This application claims the benefit under 35 U.S.C. § 119(e) of U.S. Provisional Patent Application Ser. No. 63/234,085, filed Aug. 17, 2021, the content of this related application is incorporated herein by reference in its entirety for all purposes.

### STATEMENT REGARDING FEDERALLY SPONSORED R&D

This invention was made with government support under Grant No. 2021552 awarded by the National Science Foundation. The government has certain rights in the invention.

### REFERENCE TO SEQUENCE LISTING

The present application is being filed along with a Sequence Listing in electronic format. The Sequence Listing is provided as a file entitled Sequence Listing 30KJ-302450-US, created Aug. 12, 2022, which is 33 kilobytes in size. The information in the electronic format of the Sequence Listing is incorporated herein by reference in its entirety.

### BACKGROUND

### Field

The present disclosure relates generally to the field of measurement of the states of living cells across time.

### Description of the Related Art

Living cells change over time. These dynamics of cell state underpin nearly every biological process, including development, homeostasis, disease, aging, and death. Therefore, measurement of the states of living cells across time is fundamental for biology and for addressing diverse biomedical and therapeutic challenges. Existing live-cell reporters suffer from substantial limitations. Fluorescent reporters require optical accessibility and cannot be multiplexed to simultaneously measure many aspects of cell state due to spectral overlap. Genomic recording technologies have been challenging to implement and only retain information about cells that survive to the measurement endpoint. Export and sequencing of cellular RNA could enable information-rich dynamic measurements of cell state, but previously proposed strategies rely on RNA export components of viral origin that may perturb cells, and lack barcoding schemes that provide single-cell resolution. Finally, most existing methods cannot detect the death or loss of specific cell populations over time. There is a need for compositions, methods, systems, and kits for measurement of the states of living cells across time.

### SUMMARY

Disclosed herein include compositions. In some embodiments, the composition is or comprises a nucleic acid composition. The nucleic acid composition can comprise: one or more first polynucleotide(s) encoding an RNA exporter protein; and/or one or more second polynucleotide(s) each encoding one or more reporter RNA molecule(s). In some embodiments, the RNA exporter protein comprises: an RNA-binding domain, a membrane-binding domain, and an interaction domain capable of nucleating self-assembly. In some embodiments, a plurality of RNA exporter proteins are capable of self-assembling into lipid-enveloped nanoparticles (LNs) secreted from a reporter cell in which the RNA exporter proteins are expressed, thereby generating a population of LNs comprising exported reporter RNA molecule(s).

Disclosed herein include compositions. In some embodiments, the composition is or comprises a population of reporter cells. In some embodiments, the composition comprises: a population of reporter cells comprising a nucleic acid composition disclosed herein. The population of reporter cells can comprise: one or more first polynucleotide(s) encoding an RNA exporter protein and/or one or more second polynucleotide(s) each encoding one or more reporter RNA molecule(s). In some embodiments, the RNA exporter protein comprises: an RNA-binding domain, a membrane-binding domain, and an interaction domain capable of nucleating self-assembly. In some embodiments, a plurality of RNA exporter proteins are capable of self-assembling into lipid-enveloped nanoparticles (LNs) secreted from a reporter cell in which the RNA exporter proteins are expressed, thereby generating a population of LNs comprising exported reporter RNA molecule(s).

In some embodiments, the reporter RNA molecule(s) comprise one or more barcode(s). In some embodiments, the one or more barcodes comprise reporter barcode(s) and/or cell barcode(s). In some embodiments, the reporter RNA molecule(s) each comprise packing signal(s), one or more cell barcode(s), and a reporter barcode. The composition can comprise: one or more third polynucleotide(s) each encoding one or more packing RNA molecule(s), wherein the LNs further comprise exported packing RNA molecule(s), wherein the packing RNA molecule(s) comprise a capture domain, and wherein the reporter RNA molecule(s) comprise a hybridization domain capable of hybridizing to the capture domain. In some embodiments, the reporter RNA molecule(s) each comprise a reporter barcode and one or more cell barcodes; and the packing RNA molecule(s) each comprise packing signal(s). In some embodiments, the reporter RNA molecule(s) each comprise a reporter barcode; and the packing RNA molecule(s) each comprise packing signal(s) and one or more cell barcode(s). In some embodiments, the reporter RNA molecule(s) each comprise packing signal(s) and a reporter barcode; and the packing RNA molecule(s) each comprise one or more cell barcode(s).

In some embodiments, the cell barcode(s) comprise: a clone barcode, where each reporter cell of a population of reporter cells has a single clone barcode, wherein the sequence of clone barcode is unique to each reporter cell of the population of reporter cells at an initial time point, and wherein progeny cells arising from cell division of the same reporter cell constitute a clonal population wherein each clone comprises the same clone barcode. In some embodiments, the clone barcode is static. In some embodiments, the clone barcode is selected from a library of at least about 10,000 different clone barcode sequences. In some embodiments, the cell barcode(s) comprise: a subpopulation barcode, wherein a population of reporter cells comprises one or more reporter cell subpopulations, where each reporter cell subpopulation has a single subpopulation barcode, wherein the sequence of the subpopulation barcode is unique to each reporter cell subpopulation of the population of reporter cells at an initial time point, and wherein progeny cells arising from cell division of the same reporter cell share the same subpopulation barcode. In some embodiments, the subpopulation barcode is static. In some embodiments, the subpopulation barcode is selected from a library of at least about 10,000 different subpopulation barcode sequences. In some embodiments, the cell barcode(s) comprise: a lineage barcode, where each reporter cell of a population of reporter cells has a single lineage barcode, wherein the lineage barcode is not static, wherein the lineage barcode is an editable barcode, wherein at least about 10 percent of progeny cells arising from cell division of a reporter cell have a lineage barcode different than progeny cells arising from cell division of the same reporter cell. In some embodiments, the sequence of the lineage barcode is unique to each reporter cell of the population of reporter cells at an initial time point.

In some embodiments, the clone barcode, the subpopulation barcode, and/or the lineage barcode is from about 4 nucleotides to about 30 nucleotides in length. In some embodiments, the packing RNA molecule(s) and/or reporter RNA molecule(s) are mRNA. In some embodiments, the barcode(s) and/or packing signal(s) are situated in the 5′UTR and/or 3′UTR. In some embodiments, the reporter RNA molecule(s) and/or packing RNA molecule(s) are transcribed from a RNA polymerase I promoter, a RNA polymerase II promoter, a RNA polymerase III promoter, a T7 promoter, a T3 promoter, or any combination thereof.

The composition can comprise: one or more fourth polynucleotide(s) encoding an editor and/or a targeting molecule. In some embodiments, the targeting molecule comprises single strand DNA or single strand RNA. In some embodiments, the targeting molecule comprises a guide RNA (gRNA). In some embodiments, the editor is selected from the group comprising CRISPR-Cas9, base editors, prime editors, integrases, and recombinases. In some embodiments, the editor is a base editor capable of base editing the lineage barcode. In some embodiments, said base editing comprises: adenine (A)-to-guanine (G) base editing and/or cytosine (C)- to-thymine (T) base editing. In some embodiments, the base editor comprises saCas9-KKH, Cas9-VQR, Cas9-VRQR, Cas9-VRER, Cas9-NG, ABE7.7, pNMG-624, ABE3.2, ABE5.3, pNMG-558, pNMG-576, pNMG-577, pNMG-586, ABE7.2, pNMG-620, pNMG-617, pNMG-618, pNMG-620, pNMG-621, pNGM-622, pNMG-623, ABE6.3, ABE6.4, ABE7.8, ABE7.9, ABE7.10, ABEMax, ABE8e, CP1028-ABE8e, ABE7.10-CP1041, CP1041-ABE8e, or any combination thereof. In some embodiments, the base editor comprises an adenine base editor (ABE) and/or a cytosine base editor (CBE). In some embodiments, the ABE comprises monomer and dimer versions of one or more of ABE8e, ABE8e-V106W, SaABE8e, SaKKH-ABE8e, NG-ABE8e, ABE-xCas9, ABE8e-NRTH, ABE8e-NRRH, ABE8e-NRCH, ABE8e-NG-CP1041, ABE8e-VRQR-CP1041, ABE8e-CP1041, ABE8e-CP1028, ABE8e-VRQR, ABE8e-LbCas12a (LbABE8e), ABE8e-AsCas12a (enAsABE8e), ABE8e-SpyMac, ABE8e (TadA-8e V106W), ABE8e (K20A,R21A), and ABE8e(TadA-8e V82G). In some embodiments, the lineage barcode comprises about 2 to about 40 editable units. In some embodiments, the editable units have the same length and/or sequence. In some embodiments, the lineage barcode comprises from about 2 tandem copies to about 40 tandem copies of an editable unit. In some embodiments, an editable unit is about 2 nucleotides to about 40 nucleotides in length. In some embodiments, an editable unit comprise at least 2 bases that can be converted from A to G or from C to T by the base editor, wherein the fraction of editable bases converted from A to G or from C to T by the base editor increases through time. In some embodiments, the lineage barcode comprises one or more gRNA targeting sequences, and wherein a gRNA is capable of targeting gRNA targeting sequence(s). In some embodiments, the gRNA targeting sequence is about 20 nucleotides in length. In some embodiments, each editable unit comprises a gRNA targeting sequence. In some embodiments, each editable unit comprises a Protospacer Adjacent Motif (PAM). In some embodiments, the PAM is downstream of the gRNA targeting sequence. In some embodiments, said base editing comprises base editing the lineage barcode in one reporter cell, at least one reporter cell, or each of a population of reporter cells at one or more predetermined time points. In some embodiments, said base editing comprises base editing the lineage barcode in one reporter cell, at least one reporter cell, or each of a population of reporter cells at an edit rate. In some embodiments, the edit rate is predetermined. In some embodiments, the edit rate is about 1% to about 100% edit per unit time. In some embodiments, the edit rate is about 1% to 100% edit per cell per cell division cycle.

In some embodiments, the RNA exporter protein is: a chimeric fusion protein; or a multi-subunit protein. In some embodiments, the RNA exporter protein comprises two or more components configured to dimerize via dimerization domain(s). In some embodiments, the RNA binding domain is capable of binding the packing signal(s), and wherein the packing RNA molecule(s) and/or reporter RNA molecule(s) is specifically packaged into the LNs via interaction of the packing signal(s) with the RNA-binding domain of the RNA exporter protein. In some embodiments, the abundance of reporter RNA molecule(s) exported to the exterior of a reporter cell is at least about 2-fold higher as compared to a reporter cell wherein (i) the packing signal(s) are absent from the reporter RNA molecule(s) and/or packing RNA molecule(s) and/or (ii) the RNA exporter protein does not comprise an RNA binding domain. In some embodiments, the packing signal(s) comprise an array of at least about 2, 3, 4, 5, 6, 7, 8, 9, 10, 11, 12, 13, or 14, tandem repeats of an aptamer (e.g., a MS2 stem-loop aptamer). In some embodiments, the rate of export of packing RNA molecule(s) and/or reporter RNA molecule(s) is capable of being configured by varying the number of tandem repeats.

In some embodiments, the RNA exporter protein is configured to be minimally perturbative to cellular physiology and/or minimally immunogenic. In some embodiments, the RNA exporter protein comprises or is derived from one or more components of viral origin, one or more components of a non-viral compartmentalization and secretion system, and/or one or more components of de novo designed proteins. In some embodiments, the RNA exporter protein comprises a capsid protein of viral origin, optionally fused with an RNA binding protein. In some embodiments, the one or more first polynucleotide(s) encoding the RNA exporter protein comprise packing signal(s), and wherein LNs thereby comprise RNA molecules encoding the RNA exporter protein. In some embodiments, the RNA binding domain comprises or is derived from an RNA binding protein.

In some embodiments, the packing signal(s) comprise a Ku binding hairpin and the RNA binding protein is Ku. In some embodiments, the packing signal(s) comprise a telomerase Sm7 binding motif and the RNA binding protein is Sm7. In some embodiments, the packing signal(s) comprise an MS2 phage operator stem-loop and the RNA binding protein is MS2 Coat Protein (MCP). In some embodiments, the packing signal(s) comprise a PP7 phage operator stem-loop and the RNA binding protein is PP7 Coat Protein (PCP). In some embodiments, the packing signal(s) comprise an SfMu phage Com stem-loop and the RNA binding protein is Com RNA binding protein. In some embodiments, the packing signal(s) comprise a PUF binding site (PBS) and the RNA binding protein is Pumilio/fem-3 mRNA binding factor (PUF). In some embodiments, the packing signal(s) comprise an MMLV packing signal (Psi) and the RNA binding protein is MMLV.

In some embodiments, the RNA binding domain comprises or is derived from a catalytically inactivated programmable nuclease configured to bind the packing signal(s) (e.g., catalytically inactivated CRISPR-Cas9 or -Cas13). In some embodiments, the RNA exporter protein comprises at least a portion of a viral capsid protein (e.g., a retroviral Gag protein). In some embodiments, at least a portion of the nucleocapsid domain, matrix domain, a zinc finger domain, a p1 domain, capsid domain and/or a p1-p6 domain is removed from said viral capsid protein. In some embodiments, the nucleocapsid domain is replaced with a leucine zipper (e.g., the leucine zipper of GCN4). In some embodiments, the interaction domain comprises or is derived from a viral capsid protein (e.g., a retroviral Gag protein). In some embodiments, the interaction domain comprises dimerization domain(s), e.g., leucine zipper dimerization domain from GCN4 (Zip). In some embodiments, the dimerization domain(s) comprises or is derived from SYNZIP1, SYNZIP2, SYNZIP3, SYNZIP4, SYNZIP5, SYNZIP6, SYNZIP7, SYNZIP8, SYNZIP9, SYNZIP10, SYNZIP11, SYNZIP12, SYNZIP13, SYNZIP14, SYNZIP15, SYNZIP16, SYNZIP17, SYNZIP18, SYNZIP19, SYNZIP20, SYNZIP21, SYNZIP22, SYNZIP23, BATF, FOS, ATF4, BACH1, JUND, NFE2L3, AZip, BZip, a PDZ domain ligand, an SH3 domain, a PDZ domain, a GTPase binding domain, a leucine zipper domain, an SH2 domain, a PTB domain, an FHA domain, a WW domain, a 14-3-3 domain, a death domain, a caspase recruitment domain, a bromodomain, a chromatin organization modifier, a shadow chromo domain, an F-box domain, a HECT domain, a RING finger domain, a sterile alpha motif domain, a glycine-tyrosine-phenylalanine domain, a SNAP domain, a VHS domain, an ANK repeat, an armadillo repeat, a WD40 repeat, an MH2 domain, a calponin homology domain, a Dbl homology domain, a gelsolin homology domain, a PB1 domain, a SOCS box, an RGS domain, a Toll/IL-1 receptor domain, a tetratricopeptide repeat, a TRAF domain, a Bcl-2 homology domain, a coiled-coil domain, a bZIP domain, portions thereof, variants thereof, or any combination thereof. In some embodiments, the dimerization domain(s) is a homodimerization domain or a multimerization domain (e.g., a homo- or hetero-dimerizing or multimerizing leucine zipper, a PDZ domains, a SH3 domain, aGBD domain, or any combination thereof).

In some embodiments, the RNA exporter protein self-assembles to form nanocages, and wherein the LNs comprise a plurality of said nanocages. In some embodiments, the interaction domain comprises I3-01 and/or wherein the membrane binding domain comprises rat phospholipase C delta Pleckstrin Homology domain. In some embodiments, the RNA exporter protein comprises enveloped protein nanocage domain EPN24 fused with an RNA binding protein. In some embodiments, the RNA exporter protein comprises: a myristoylation motif of HIV-1 NL4-3 Gag, optionally amino acid residues 2-6; a myristoylation/palmitoylation motif of Lyn kinase, optionally amino acid residues 2-13; a Pleckstrin Homology domain of rat phospholipase Cδ; and/or a p6 domain of HIV-1 NL4-3 Gag. In some embodiments, the RNA exporter protein is configured to operate in a cell type and/or species different than the cell type and/or species in which at least one parental component of said RNA exporter protein operates. In some embodiments, the RNA exporter protein is a species-chimeric RNA exporter protein. In some embodiments, the RNA exporter protein comprises a chimeric viral capsid protein wherein the matrix domain is replaced with the matrix domain of another virus. In some embodiments, the RNA exporter protein comprises an amino acid sequence that is at least 80%, 85%, 90%, 95%, 98%, 99%, or 100% identical to any one of SEQ ID NOS: 1-24. In some embodiments, the expression of the RNA exporter protein in the reporter cell(s) does not alter the endogenous transcriptome, morphology, and/or physiology of said reporter cell(s). In some embodiments, the RNA exporter protein is constitutively expressed by the reporter cell(s).

In some embodiments, the population of reporter cells comprises a plurality of reporter cells that differ with respect to cell type and/or cell state. In some embodiments, the one or more reporter RNA molecule(s) comprise a plurality of reporter RNA molecules. In some embodiments, each of the reporter RNA molecules comprises a unique reporter barcode indicating a unique cell type and/or a unique cell state of the reporter cell from which it is derived. In some embodiments, the plurality of reporter RNA molecules comprise at least about 2, about 10, about 20, about 30, about 40, or about 50, different reporter RNA molecules each comprising a unique reporter barcode. In some embodiments, the presence and/or amount of an exported reporter RNA molecule comprising a unique reporter barcode is correlated with the presence and/or amount of the unique cell type and/or a unique cell state in said reporter cell. In some embodiments, the degree of expression and/or degradation of the plurality of reporter RNA molecules is associated with the presence and/or amount of the unique cell type and/or a unique cell state. In some embodiments, one or more second promoter(s) are operably linked to each of the one or more second polynucleotide(s) each encoding one or more reporter RNA molecule(s). In some embodiments, a second promoter is capable of inducing transcription of a second polynucleotide to generate a reporter RNA molecule comprising a unique reporter barcode depending on the presence and/or amount of a unique cell type and/or a unique cell state associated with said unique reporter barcode. In some embodiments, second promoter(s) are transcription factor-dependent, signal-responsive, metabolic, and/or circadian promoters. In some embodiments, the one or more second polynucleotide(s) comprise one or more silencer effector binding sequences. In some embodiments, the silencer effector comprises a microRNA (miRNA), a precursor microRNA (pre-miRNA), a small interfering RNA (siRNA), a short-hairpin RNA (shRNA), precursors thereof, derivatives thereof, or a combination thereof. In some embodiments, said silencer effector is capable of binding the one or more silencer effector binding sequences, thereby reducing the stability of reporter RNA molecule(s). In some embodiments, the expression and/or activity of the silencer effector configured to be responsive to changes in cell state and/or cell type. In some embodiments, the reporter cell comprises a circuit configured to regulate the expression and/or stability of the plurality of reporter RNA molecules in response to the cell type and/or cell state of the reporter cell, optionally said circuit comprises one or more components encoded by one or more fifth polynucleotide(s).

In some embodiments, a unique cell type and/or a unique cell state comprises a unique gene expression pattern, e.g. the unique cell type and/or unique cell state comprises a unique anatomic location. In some embodiments, the unique cell type and/or the unique cell state comprises anatomically locally unique gene expression. In some embodiments, a unique cell type and/or a unique cell state is caused by hereditable, environmental, and/or idiopathic factors. In some embodiments, the unique cell type and/or the cell in the unique cell state (i) causes and/or aggravates a disease or disorder and/or (ii) is associated with the pathology of a disease or disorder. In some embodiments, the unique cell state comprises a senescent cell state induced by a tumor microenvironment. In some embodiments, the senescent cell state induced by a tumor microenvironment comprises expression of CD57, KRLG1, TIGIT, or any combination thereof. In some embodiments, the unique cell state and/or unique cell type is characterized by aberrant signaling of one or more signal transducer(s). In some embodiments, the unique cell state comprises: a physiological state (e.g., a cell cycle state, a differentiation state, a development state a metabolic state, or a combination thereof); and/or a pathological state (e.g., a disease state, a human disease state, a diabetic state, an immune disorder state, a neurodegenerative disorder state, an oncogenic state, or a combination thereof).

In some embodiments, the unique cell state and/or unique cell type is characterized by one or more of cell proliferation, stress pathways, oxidative stress, stress kinase activation, DNA damage, lipid metabolism, carbohydrate regulation, metabolic activation including Phase I and Phase II reactions, Cytochrome P-450 induction or inhibition, ammonia detoxification, mitochondrial function, peroxisome proliferation, organelle function, cell cycle state, morphology, apoptosis, DNA damage, metabolism, signal transduction, cell differentiation, cell-cell interaction and cell to non-cellular compartment.

In some embodiments, the unique cell state and/or unique cell type is characterized by one or more of acute phase stress, cell adhesion, AH-response, anti-apoptosis and apoptosis, antimetabolism, anti-proliferation, arachidonic acid release, ATP depletion, cell cycle disruption, cell matrix disruption, cell migration, cell proliferation, cell regeneration, cell-cell communication, cholestasis, differentiation, DNA damage, DNA replication, early response genes, endoplasmic reticulum stress, estogenicity, fatty liver, fibrosis, general cell stress, glucose deprivation, growth arrest, heat shock, hepatotoxicity, hypercholesterolemia, hypoxia, immunotox, inflammation, invasion, ion transport, liver regeneration, cell migration, mitochondrial function, mitogenesis, multidrug resistance, nephrotoxicity, oxidative stress, peroxisome damage, recombination, ribotoxic stress, sclerosis, steatosis, teratogenesis, transformation, disrupted translation, transport, and tumor suppression.

In some embodiments, the unique cell state and/or unique cell type is characterized by one or more of nutrient deprivation, hypoxia, oxidative stress, hyperproliferative signals, oncogenic stress, DNA damage, ribonucleotide depletion, replicative stress, and telomere attrition, promotion of cell cycle arrest, promotion of DNA-repair, promotion of apoptosis, promotion of genomic stability, promotion of senescence, and promotion of autophagy, regulation of cell metabolic reprogramming, regulation of tumor microenvironment signaling, inhibition of cell stemness, survival, and invasion.

In some embodiments, the cell type is: an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof. In some embodiments, the stem cell comprises an embryonic stem cell, an induced pluripotent stem cell (iPSC), a hematopoietic stem/progenitor cell (HSPC), or any combination thereof.

In some embodiments, the reporter cells comprise cells situated in an organ and/or tissue, e.g., an organ and/or tissue or a subject (e.g., different organs and/or tissues of a subject). In some embodiments, the LNs contacted with RNase are capable of protecting reporter RNA molecule(s) comprised therein from RNase-mediated degradation (e.g., in the absence of detergent). In some embodiments, the average diameter of the LNs of the population of LNs is about 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, 205 nm, 210 nm, 215 nm, 220 nm, 225 nm, 230 nm, 235 nm, 240 nm, 245 nm, 250 nm, 300 nm, 400 nm, or 500 nm. In some embodiments, the average is the mean (e.g., arithmetic mean, geometric mean, and/or harmonic mean), median or mode. In some embodiments, the LNs have a minimum diameter and/or a maximum diameter of about 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, 205 nm, 210 nm, 215 nm, 220 nm, 225 nm, 230 nm, 235 nm, 240 nm, 245 nm, 250 nm, 300 nm, 400 nm, or 500 nm. In some embodiments, the diameter is hydrodynamic diameter, e.g., as measured by dynamic light scattering (DLS).

In some embodiments, the population of LNs differ from each other with respect the RNA contents. In some embodiments, the LNs comprise a lipid bilayer (e.g., a lipid bilayer derived from the reporter cell from which the LN was secreted). In some embodiments, the reporter cells are situated in a tissue. In some embodiments, reporter RNA molecule(s) encode a dosage indicator protein. In some embodiments, the dosage indicator protein is detectable. In some embodiments, the dosage indicator protein comprises green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCherry, mruby3, rsCherry, rsCherryRev, derivatives thereof, or any combination thereof. In some embodiments, the LNs comprise exported endogenous RNA molecule(s). In some embodiments, the endogenous RNA molecule(s) comprise one or more of messenger RNAs (mRNAs), microRNAs, small interfering RNAs (siRNAs), and RNA degradation products. In some embodiments, the endogenous RNA molecule(s) are not mitochondrial RNA molecules. In some embodiments, the exported endogenous RNA molecule(s) comprise an unbiased sample of the non-mitochondrial transcriptome of the reporter cell. In some embodiments, the packing signal(s) comprise an array of at least about 2, 3, 4, 5, 6, 7, 8, 9, 10, 11, 12, 13, or 14, tandem repeats of an aptamer (e.g., a MS2 stem-loop aptamer). In some embodiments, the relative abundance of endogenous RNA molecules to reporter RNA molecule(s) in secreted LNs can be configured by varying the number of tandem repeats. Disclosed herein include pharmaceutical compositions. In some embodiments, the pharmaceutical composition comprises: a composition provided herein (e.g., a nucleic acid composition for generating a population of reporter cells), wherein the pharmaceutical composition further comprises one or more pharmaceutically acceptable carriers, diluents and/or excipients.

In some embodiments, one or more of the first polynucleotide(s), the second polynucleotide(s), the third polynucleotide(s), the fourth polynucleotide(s), and the fifth polynucleotide(s), are operably connected to a promoter selected from the group comprising: a minimal promoter, optionally TATA, miniCMV, and/or miniPromo; a ubiquitous promoter; a tissue-specific promoter and/or a lineage-specific promoter; and/or a ubiquitous promoter, optionally a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CASI promoter, a CBH promoter, or any combination thereof.

In some embodiments, the nucleic acid composition is complexed or associated with one or more lipids or lipid-based carriers, thereby forming liposomes, lipid nanoparticles (LNPs), lipoplexes, and/or nanoliposomes, optionally encapsulating the nucleic acid composition. In some embodiments, the nucleic acid composition is, comprises, or further comprises, one or more vectors. In some embodiments, at least one of the one or more vectors is a viral vector, a plasmid, a transposable element, a naked DNA vector, a lipid nanoparticle (LNP), or any combination thereof. In some embodiments, the viral vector is an AAV vector, a lentivirus vector, a retrovirus vector, an adenovirus vector, a herpesvirus vector, a herpes simplex virus vector, a cytomegalovirus vector, a vaccinia virus vector, a MVA vector, a baculovirus vector, a vesicular stomatitis virus vector, a human papillomavirus vector, an avipox virus vector, a Sindbis virus vector, a VEE vector, a Measles virus vector, an influenza virus vector, a hepatitis B virus vector, an integration-deficient lentivirus (IDLV) vector, or any combination thereof. In some embodiments, the transposable element is piggybac transposon or sleeping beauty transposon. In some embodiments, the polynucleotide(s) encoding the RNA exporter protein, the reporter RNA molecule(s), and/or the packing RNA molecule(s) are comprised in the one or more vectors. In some embodiments, the polynucleotide(s) encoding the RNA exporter protein, the reporter RNA molecule(s), and/or the packing RNA molecule(s) are comprised in the same vector and/or different vectors. In some embodiments, the polynucleotide(s) encoding the RNA exporter protein, the reporter RNA molecule(s), and/or the packing RNA molecule(s) are situated on the same nucleic acid and/or different nucleic acids. In some embodiments, the RNA exporter protein is configured to exhibit minimal immunogenicity in a subject, e.g., derived from commensal viruses or endogenous viruses, and/or novo designed proteins and/or humanized.

Disclosed herein include systems for export of reporter RNA molecules. In some embodiments, the kit comprises: an RNA exporter protein provided herein; and one or more reporter RNA molecule(s) disclosed herein. Disclosed herein include systems for non-destructive live continuous cell measurement of cell state and/or cell type. In some embodiments, the kit comprises: an RNA exporter protein provided herein; and one or more reporter RNA molecule(s) disclosed herein. The system can comprise: one or more packing RNA molecule(s) disclosed herein.

Disclosed herein include populations of lipid-enveloped nanoparticles (LNs). In some embodiments, the population of LNs comprise: an RNA exporter protein provided herein; and one or more reporter RNA molecule(s) disclosed herein. The population of LNs can comprise: one or more packing RNA molecule(s) disclosed herein. The population of LNs can be derived from expression of a nucleic acid composition disclosed herein.

Disclosed herein include methods for determining the cell type and/or cell state of one or more reporter cells. In some embodiments, the method comprises: providing a population of reporter cells provided herein; isolating a plurality of exported reporter RNA molecule(s) at one or more time points; and obtaining sequence information of the plurality of exported reporter RNA molecule(s), or products thereof, to determine the cell type and/or cell state of the reporter cell(s).

The method can comprise: isolating a plurality of exported packing RNA molecule(s) at one or more time points; and obtaining sequence information of the plurality of exported packing RNA molecule(s), or products thereof, to determine the cell type and/or cell state of the reporter cell(s).

In some embodiments, the providing step comprises incubation of the reporter cell(s). In some embodiments, one or more reporter cell(s) divide and/or die during said incubation. In some embodiments, the providing step comprises introducing into the reporter cell(s) nucleic acid composition(s) comprising the one or more first polynucleotide(s), the one or more second polynucleotide(s), the one or more third polynucleotide(s), the one or more fourth polynucleotide(s), and/or the one or more fifth polynucleotide(s). In some embodiments, the one or more first polynucleotide(s), the one or more second polynucleotide(s), the one or more third polynucleotide(s), the one or more fourth polynucleotide(s), and/or the one or more fifth polynucleotide(s) become integrated in the genome of the reporter cell(s) after the introducing step. In some embodiments, providing the reporter cells comprises transducing reporter cells with a library of vectors encoding one or more cell barcode(s) at a multiplicity of infection (MOI) configured to increase the likelihood that each cell is transduced with only a single vector, e.g., an MOI between about 0.3 and 0.75. In some embodiments, each of the library members of the library of vectors encodes cell barcode(s) of a different sequence.

In some embodiments, the method comprises exposing the reporter cell(s) to one or more agents before, during, and/or after the one or more time points. In some embodiments, the one or more agents comprise: (i) one or more of a chemical agent, a pharmaceutical, small molecule, a biologic, a CRISPR single-guide RNA (sgRNA), a small interfering RNA (siRNA), CRISPR RNA (crRNA), a small hairpin RNA (shRNA), a microRNA (miRNA), a piwi-interacting RNA (piRNA), an antisense oligonucleotide, a peptide or peptidomimetic inhibitor, an aptamer, an antibody, an intrabody, or any combination thereof; (ii) an expression vector, wherein the expression vector encodes one or more of the following: an mRNA, an antisense nucleic acid molecule, a RNAi molecule, a shRNA, a mature miRNA, a pre-miRNA, a pri-miRNA, an anti-miRNA, a ribozyme, any combination thereof; (iii) an infectious agent, an anti-infectious agent, or a mixture thereof; (iv) a cytotoxic agent (e.g., a chemotherapeutic agent, a biologic agent, a toxin, a radioactive isotope, or any combination thereof); (v) one or more of an epigenetic modifying agent, epigenetic enzyme, a bicyclic peptide, a transcription factor, a DNA or protein modification enzyme, a DNA-intercalating agent, an efflux pump inhibitor, a nuclear receptor activator or inhibitor, a proteasome inhibitor, a competitive inhibitor for an enzyme, a protein synthesis inhibitor, a nuclease, a protein fragment or domain, a tag or marker, an antigen, an antibody or antibody fragment, a ligand or a receptor, a synthetic or analog peptide from a naturally-bioactive peptide, an anti-microbial peptide, a pore-forming peptide, a targeting or cytotoxic peptide, a degradation or self-destruction peptide, a CRISPR component system or component thereof, DNA, RNA, artificial nucleic acids, a nanoparticle, an oligonucleotide aptamer, a peptide aptamer, or any combination thereof; and/or (vi) at least one effector activity selected from the group consisting of: modulating a biological activity, binding a regulatory protein, modulating enzymatic activity, modulating substrate binding, modulating receptor activation, modulating protein stability/degradation, modulating transcript stability/degradation, or any combination thereof.

In some embodiments, the isolating step comprises isolating LNs and/or extracting RNA to generate a plurality of RNA molecules. In some embodiments, the isolating step comprises: separating cells from the extracellular environment to generate a supernatant; clarifying the supernatant; and extracting RNA from the supernatant to generate a plurality of RNA molecules. In some embodiments, clarifying the supernatant comprises: centrifuging the supernatant (e.g., centrifugation at 3000 g for 5 minutes); and/or filtering the supernatant through a filter (e.g., a 0.45 um filter).

The method can comprise: contacting a first strand primer with the plurality of RNA molecules, optionally the first strand primer is a target-specific primer, further optionally the target-specific primer comprises oligo(dT) and/or a first universal sequence. The method can comprise: conducting a first strand synthesis reaction using a reverse transcriptase to generate a plurality of first strand synthesis products. In some embodiments, obtaining sequence information comprises obtaining sequence information of the plurality of first strand synthesis products, or products thereof. The method can comprise: amplifying the plurality of first strand synthesis products using a first amplification primer and a second amplification primer, thereby generating a plurality of amplicons. In some embodiments, obtaining sequence information comprises obtaining sequence information of the plurality of amplicons, or products thereof. In some embodiments, amplifying the plurality of first strand synthesis products comprises adding sequences of binding sites of sequencing primers and/or sequencing adaptors, complementary sequences thereof, and/or portions thereof, to the plurality of first strand synthesis products. In some embodiments, the sequencing adaptors comprise a P5 sequence, a P7 sequence, complementary sequences thereof, and/or portions thereof. In some embodiments, the sequencing primers comprise a Read 1 sequencing primer, a Read 2 sequencing primer, complementary sequences thereof, and/or portions thereof. In some embodiments, the first amplification primer and the second amplification primer are target-specific primers. In some embodiments, the first amplification primer is capable of hybridizing to the first universal sequence, or a complement thereof, and the second amplification primer is a target-specific primer. In some embodiments, the target-specific primers are configured to amplify the reporter barcode(s) and/or cell barcode(s).

The method can comprise: adding spike-in RNA molecules of known quantity to the supernatant, extracted RNA, or isolated LNs; and obtaining sequence information of the spike-in RNA molecules, or products thereof, to determine relative abundance of exported reporter RNA molecule(s) and/or endogenous RNA molecule(s) across samples of reporter cells. In some embodiments, the spike-in RNA molecules are not homologous to genomic sequences of the reporter cell(s). In some embodiments, spike-in RNA molecules are homologous to genomic sequences of a species, optionally the species is a non-mammalian species. In some embodiments, the non-mammalian species is a phage species, optionally said phage species is T7 phage, a PhiX phage, or any combination thereof.

In some embodiments, obtaining sequence information comprises: obtaining sequencing data comprising a plurality of sequencing reads of the plurality of exported reporter RNA molecule(s), the plurality of exported package RNA molecule(s), the plurality of first strand synthesis products, the plurality of amplicons, and/or products thereof, wherein each of the plurality of sequencing reads comprise at least one cell barcode sequence, and a reporter barcode sequence.

The method can comprise: for each unique lineage barcode sequence, which indicates a single reporter cell of the population of reporter cells: detecting the presence and/or amount of each reporter barcode of the plurality of reporter barcodes, thereby generating a profile of each reporter cell, wherein the profile comprises collective cell type(s) and cell state(s) of said reporter cell. In some embodiments, the method further comprises performing phylogenetic reconstruction to determine lineage dynamics.

The method can comprise: for each unique subpopulation barcode sequence, which indicates a single subpopulation of reporter cells of the population of reporter cells: detecting the presence and/or amount of each reporter barcode of the plurality of reporter barcodes, thereby generating a profile of each subpopulation, wherein the profile comprises collective cell type(s) and cell state(s) of said subpopulation.

The method can comprise: for each unique clone barcode sequence, which indicates a clonal population of the population of reporter cells: detecting the presence and/or amount of each reporter barcode of the plurality of reporter barcodes, thereby generating a profile of each clonal population, wherein the profile comprises collective cell type(s) and cell state(s) of said clonal population.

In some embodiments, the one or more time points comprise a plurality of time points. In some embodiments, the method comprises longitudinally monitoring single-cell state dynamics, subpopulation dynamics, clonal population dynamics, and/or lineage dynamics, optionally one or more lineages do not exist at the last time point. In some embodiments, the method comprises further identifying genetic or molecular perturbations which affect single-cell state dynamics, subpopulation dynamics, clonal population dynamics, and/or lineage dynamics. In some embodiments, the genetic or molecular perturbations are associated with a disease or disorder, or treatment thereof.

In some embodiments, the LNs comprise exported endogenous RNA molecule(s) and further comprise reporter RNA molecule(s) and/or packing RNA molecule(s) comprising cell barcode(s). In some embodiments, the method comprises physically linking said cell barcode(s) to said endogenous RNA molecule(s), e.g., via ligation, polymerization, primer extension, or physical co-compartmentalization. The method can comprise: obtaining sequence information of the endogenous RNA molecule(s), or product thereof. In some embodiments, obtaining sequence information comprises: immobilizing individual LNs of the population of LNs on a surface; and performing fluorescence in situ hybridization or in situ sequencing. In some embodiments, obtaining sequence information of the endogenous RNA molecule(s), or products thereof, comprises: obtaining sequencing data comprising a plurality of sequencing reads of the endogenous RNA molecule(s), or products thereof, wherein each of the plurality of sequencing reads comprise at least one cell barcode sequence, and a sequence of at least a portion of an endogenous RNA molecule.

The method can comprise: for each unique lineage barcode sequence, which indicates a single reporter cell of the population of reporter cells: detecting the presence and/or amount of endogenous RNA molecules, thereby generating a transcriptomic profile of each reporter cell. The method can comprise: for each unique subpopulation barcode sequence, which indicates a single subpopulation of reporter cells of the population of reporter cells: detecting the presence and/or amount of endogenous RNA molecules, thereby generating a transcriptomic profile of each subpopulation. The method can comprise: for each unique clone barcode sequence, which indicates a clonal population of the population of reporter cells: detecting the presence and/or amount of endogenous RNA molecules, thereby generating a transcriptomic profile of each clonal population. In some embodiments, the one or more time points comprise a plurality of time points, and the method comprises longitudinally monitoring single-cell transcriptome dynamics.

Disclosed herein include methods for generating an enriched population of lipid-enveloped nanoparticles (LNs). In some embodiments, the method comprises: providing a population of reporter cells provided herein, wherein the reporter cells secrete LNs comprising an affinity tag; and enriching the LNs using the affinity tag, optionally via a column, bead, and/or continuous flow. In some embodiments, the LNs comprise an affinity tag; and the method comprises enriching the LNs using the affinity tag, optionally via a column, bead, and/or continuous flow. In some embodiments, the affinity tag is present on the surface of the LNs, the affinity tag is fused to the RNA exporter protein or the affinity tag is separate from the RNA exporter protein, and/or the affinity tag is selected from the group comprising biotin, azido group, acetylene group, HIS-tag, Calmodulin-tag, CBP, CYD, Strep II, FLAG-tag, HA-tag, Myc-tag, S-tag, SBP-tag, Softag-1, Softag-3, V5-tag, Xpress-tag, Isopeptag, SpyTag, B, HPC peptide tags, GST, MBP, biotin carboxyl carrier protein, glutathione-S-transferase-tag, green fluorescent protein-tag, maltose binding protein-tag, Nus-tag, Strep-tag, and thioredoxin-tag.

### BRIEF DESCRIPTION OF THE DRAWINGS

FIGS. 1A-1H depict non-limiting exemplary schematics and data related to engineered viral RNA exporters efficiently and specifically packaging, secreting, and protecting RNA. FIG. 1A depicts a non-limiting exemplary schematic of non-destructive measurement cell population and state dynamics. FIG. 1B depicts non-limiting exemplary schematics of viral RNA exporter designs. Left, exporter based on Moloney Murine Leukemia Virus (MMLV) Gag capsid protein. FIG. 1C depicts data related to negative stain transmission electron microscopy showing secretion of enveloped particles from HEK293T cells transfected with plasmids encoding RNA exporters. FIG. 1D depicts data related to the efficiency and specificity of RNA export to supernatant determined by reverse transcription followed by quantitative PCR (RT-qPCR). FIG. 1E depicts a non-limiting exemplary schematic of an experiment to characterize RNA exporters by sequencing. FIG. 1F depicts data related to the specificity of RNA export determined by sequencing. FIG. 1G depicts data related to the quantification of endogenous cellular RNA among supernatant RNA. FIG. 1H depicts data related to RNA exporters packaging and protecting RNA from degradation.

FIGS. 2A-2D depict non-limiting exemplary schematics and data related to the design and characterization of engineered viral RNA exporters. FIG. 2A depicts non-limiting exemplary domain architectures of viral RNA exporters. Left to right, N- to C-terminus. Colors indicate distinct protein domains. FIG. 2B depicts data related to the quantification of RNA export by viral RNA exporters with various architectures using reverse transcription followed by quantitative PCR (RT-qPCR). FIG. 2C depicts data related to the quantification of RNA export system components in cellular RNA by RT-qPCR, using the same specimens as (B). FIG. 2D depicts data related to technical characteristics of RT-qPCR assay for RNA export to supernatant, indicating that assay faithfully and reproducibly measures RNA, not DNA, abundance.

FIGS. 3A-3G depict non-limiting exemplary schematics and data related to synthetic non-viral RNA exporters efficiently and specifically packaging, secreting, and protecting RNA. FIG. 3A depicts a non-limiting exemplary schematic of synthetic non-viral RNA exporter design. FIG. 3B depicts a non-limiting exemplary protein design model of an RNA-exporting nanocage. FIG. 3C depicts data related to negative stain transmission electron microscopy showing secretion of enveloped particles from HEK293T cells transfected with plasmids encoding RNA exporter. FIG. 3D depicts data related to the efficiency and specificity of RNA export to supernatant determined by reverse transcription followed by quantitative PCR (RT-qPCR). FIG. 3E depicts data related to the specificity of RNA export determined by sequencing. FIG. 3F depicts data related to the quantification of endogenous cellular RNA among supernatant RNA showing specific RNA export, similar to engineered viral RNA exporters. FIG. 3G depicts data related to the RNA exporters packaging and protecting RNA from degradation.

FIGS. 4A-4C depict non-limiting exemplary schematics and data related to the design and characterization of synthetic RNA exporters. FIG. 4A depicts non-limiting exemplary domain architectures of synthetic RNA exporters. FIG. 4B depicts a non-limiting exemplary schematic of assay for RNA export using stable reporter cell line. FIG. 4C depicts data related to the quantification of RNA export by synthetic RNA exporters with various architectures using RT-qPCR.

FIGS. 5A-5E depict data related to physical and functional features of RNA export systems. FIG. 5A depicts data related to the quantification of RNA export by RT-qPCR revealing that the rate of RNA export depends on copy number of the packaging signal, which is a tandem array of MS2 aptamers. FIG. 5B depicts data related to the quantification of RNA export by RT-qPCR reveals that RNA exporters can package their own RNA. FIG. 5C depicts data related to negative stain transmission electron microscopy showing the lack of enveloped particles in supernatant collected from HEK293T cells transfected with plasmids encoding the reporter, but not the exporter. FIG. 5D depicts data related to the physical size of RNA export particles, as determined by dynamic light scattering. FIG. 5E depicts data related to the quantification of RNA export by RT-qPCR from mouse embryonic stem cells (mESCs).

FIGS. 6A-6C depict data showing RNA exporters are non-toxic and do not perturb cellular morphology and transcriptome. FIG. 6A depicts non-limiting exemplary images of HEK293T cells transfected with RNA exporters and reporters. FIG. 6B depicts data related to the quantification of toxicity of RNA exporter expression in HEK293T cells using flow cytometry with dead stain (ethidium homodimer-1). FIG. 6C depicts data related to differential expression analysis of cellular transcriptomes of cells transfected with and without exporters.

FIGS. 7A-7F depict non-limiting exemplary schematics and data related to RNA export enabling continuous monitoring of mammalian cell population dynamics. FIG. 7A depicts a non-limiting exemplary schematic of monitoring cell population dynamics using exported RNA barcodes. FIG. 7B depicts a non-limiting exemplary design and construction of exportable RNA clone barcodes. FIG. 7C depicts a non-limiting exemplary schematic of an experiment to measure population dynamics. FIG. 7D depicts data related to the fidelity of clone abundance measured by exported RNA reporter system. FIG. 7E depicts data related to the reproducibility of clone abundance measured by exported RNA reporter system. FIG. 7F depicts data related to clone population dynamics revealed by exported RNA reporter system.

FIGS. 8A-8F depict non-limiting exemplary schematics and data related to RNA export enabling continuous monitoring of mammalian cell lineage dynamics. FIG. 8A depicts a non-limiting exemplary schematic of cell lineage illustrating changes across time, including birth and death events, and that measurements at the very end of an experiment (endpoint) can yield incomplete lineage histories. FIG. 8B depicts a non-limiting exemplary schematic of using RNA export of editable barcodes to continuously monitor cell lineage history. FIG. 8C depicts a non-limiting exemplary schematic of construction of cell lines for edit and export of lineage barcodes.

FIG. 8D depicts a non-limiting exemplary design of editable exportable barcode. FIG. 8E depicts a non-limiting exemplary schematic of an experiment to monitor cell lineage dynamics. FIG. 8F depicts data related to the quantification of lineage barcode editing across time.

FIG. 9 depicts a non-limiting exemplary schematic of a technique to non-destructively monitor single-cell transcriptome dynamics using export of RNA.

FIGS. 10A-10D depict data related to transcriptomic characterization of RNA export. FIG. 10A depicts data related to the quantification of the absolute abundance of reporter RNA in supernatant. FIG. 10B depicts data related to the quantification of the abundance of reporter and endogenous genes in supernatant of cells transfected with various exporters. FIG. 10C depicts data related to the abundance of endogenous genes and transgenes in supernatant of cells transfected with and without exporter. FIG. 10D depicts data related to the relative abundance of endogenous genes and transgenes in RNA from cells and supernatant.

FIGS. 11A-11C depict data related to characteristics of barcode library used for clone labeling. FIG. 11A depicts a non-limiting exemplary graph related to diversity of barcodes. FIG. 11B depicts a non-limiting exemplary graph related to estimation of total diversity of barcode libraries based on capture-recapture statistics. FIG. 11C depicts a non-limiting exemplary graph related to labeling capacity of barcode libraries based on the collision rate within samples of varying size.

FIGS. 12A-12D depict non-limiting exemplary schematics and data related to characteristics and performance of measurement of clone dynamics using RNA export and sequencing. FIG. 12A depicts a non-limiting exemplary schematic of experimental workflow for measuring clone abundances by RNA export and sequencing. FIG. 12B depicts data related to the quantification of clone abundances in replicate samples of cellular RNA. FIG. 12C depicts data related to rarefaction analysis of sequencing depth. Results show that clone discovery is saturated at <1M reads/sample. Related to experiment shown in FIG. 7F. FIG. 12D depicts data related to distributions of clone growth rates based on changes in abundance from one timepoint (t) to the next timepoint (t+1) in well containing puromycin.

FIG. 13 depicts a non-limiting exemplary schematic of the methods, compositions, and systems provided herein.

FIG. 14 depicts a non-limiting schematic related to measurement of cell barcodes by sequencing of exported RNA reporter.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include compositions. In some embodiments, the composition is or comprises a nucleic acid composition. The nucleic acid composition can comprise: one or more first polynucleotide(s) encoding an RNA exporter protein; and/or one or more second polynucleotide(s) each encoding one or more reporter RNA molecule(s). In some embodiments, the RNA exporter protein comprises: an RNA-binding domain, a membrane-binding domain, and an interaction domain capable of nucleating self-assembly. In some embodiments, a plurality of RNA exporter proteins are capable of self-assembling into lipid-enveloped nanoparticles (LNs) secreted from a reporter cell in which the RNA exporter proteins are expressed, thereby generating a population of LNs comprising exported reporter RNA molecule(s).

Disclosed herein include compositions. In some embodiments, the composition is or comprises a population of reporter cells. In some embodiments, the composition comprises: a population of reporter cells comprising a nucleic acid composition disclosed herein. The population of reporter cells can comprise: one or more first polynucleotide(s) encoding an RNA exporter protein and/or one or more second polynucleotide(s) each encoding one or more reporter RNA molecule(s). In some embodiments, the RNA exporter protein comprises: an RNA-binding domain, a membrane-binding domain, and an interaction domain capable of nucleating self-assembly. In some embodiments, a plurality of RNA exporter proteins are capable of self-assembling into lipid-enveloped nanoparticles (LNs) secreted from a reporter cell in which the RNA exporter proteins are expressed, thereby generating a population of LNs comprising exported reporter RNA molecule(s).

Disclosed herein include systems for export of reporter RNA molecules. In some embodiments, the kit comprises: an RNA exporter protein provided herein; and one or more reporter RNA molecule(s) disclosed herein. Disclosed herein include systems for non-destructive live continuous cell measurement of cell state and/or cell type. In some embodiments, the kit comprises: an RNA exporter protein provided herein; and one or more reporter RNA molecule(s) disclosed herein. The system can comprise: one or more packing RNA molecule(s) disclosed herein.

Disclosed herein include populations of lipid-enveloped nanoparticles (LNs). In some embodiments, the population of LNs comprise: an RNA exporter protein provided herein; and one or more reporter RNA molecule(s) disclosed herein. The population of LNs can comprise: one or more packing RNA molecule(s) disclosed herein. The population of LNs can be derived from expression of a nucleic acid composition disclosed herein.

Disclosed herein include methods for determining the cell type and/or cell state of one or more reporter cells. In some embodiments, the method comprises: providing a population of reporter cells provided herein; isolating a plurality of exported reporter RNA molecule(s) at one or more time points; and obtaining sequence information of the plurality of exported reporter RNA molecule(s), or products thereof, to determine the cell type and/or cell state of the reporter cell(s).

The method can comprise: isolating a plurality of exported packing RNA molecule(s) at one or more time points; and obtaining sequence information of the plurality of exported packing RNA molecule(s), or products thereof, to determine the cell type and/or cell state of the reporter cell(s).

Disclosed herein include methods for generating an enriched population of lipid-enveloped nanoparticles (LNs). In some embodiments, the method comprises: providing a population of reporter cells provided herein, wherein the reporter cells secrete LNs comprising an affinity tag; and enriching the LNs using the affinity tag, optionally via a column, bead, and/or continuous flow.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Spring Harbor Press (Cold Spring Harbor, N.Y. 1989). For purposes of the present disclosure, the following terms are defined below.

As used herein, the terms “nucleic acid” and “polynucleotide” are interchangeable and refer to any nucleic acid, whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sultone linkages, and combinations of such linkages. The terms “nucleic acid” and “polynucleotide” also specifically include nucleic acids composed of bases other than the five biologically occurring bases (adenine, guanine, thymine, cytosine and uracil).

The term “vector” as used herein, can refer to a vehicle for carrying or transferring a nucleic acid. Non-limiting examples of vectors include plasmids and viruses (for example, AAV viruses).

The term “construct,” as used herein, refers to a recombinant nucleic acid that has been generated for the purpose of the expression of a specific nucleotide sequence(s), or that is to be used in the construction of other recombinant nucleotide sequences.

As used herein, the term “plasmid” refers to a nucleic acid that can be used to replicate recombinant DNA sequences within a host organism. The sequence can be a double stranded DNA.

The term “element” refers to a separate or distinct part of something, for example, a nucleic acid sequence with a separate function within a longer nucleic acid sequence. The term “regulatory element” and “expression control element” are used interchangeably herein and refer to nucleic acid molecules that can influence the expression of an operably linked coding sequence in a particular host organism. These terms are used broadly to and cover all elements that promote or regulate transcription, including promoters, core elements required for basic interaction of RNA polymerase and transcription factors, upstream elements, enhancers, and response elements (see, e.g., Lewin, “Genes V” (Oxford University Press, Oxford) pages 847-873). Exemplary regulatory elements in prokaryotes include promoters, operator sequences and a ribosome binding sites. Regulatory elements that are used in eukaryotic cells can include, without limitation, transcriptional and translational control sequences, such as promoters, enhancers, splicing signals, polyadenylation signals, terminators, protein degradation signals, internal ribosome-entry element (IRES), 2A sequences, and the like, that provide for and/or regulate expression of a coding sequence and/or production of an encoded polypeptide in a host cell.

As used herein, the term “promoter” is a nucleotide sequence that permits binding of RNA polymerase and directs the transcription of a gene. Typically, a promoter is located in the 5′ non-coding region of a gene, proximal to the transcriptional start site of the gene. Sequence elements within promoters that function in the initiation of transcription are often characterized by consensus nucleotide sequences. Examples of promoters include, but are not limited to, promoters from bacteria, yeast, plants, viruses, and mammals (including humans). A promoter can be inducible, repressible, and/or constitutive. Inducible promoters initiate increased levels of transcription from DNA under their control in response to some change in culture conditions, such as a change in temperature.

As used herein, the term “enhancer” refers to a type of regulatory element that can increase the efficiency of transcription, regardless of the distance or orientation of the enhancer relative to the start site of transcription.

As used herein, the term “operably linked” is used to describe the connection between regulatory elements and a gene or its coding region. Typically, gene expression is placed under the control of one or more regulatory elements, for example, without limitation, constitutive or inducible promoters, tissue-specific regulatory elements, and enhancers. A gene or coding region is said to be “operably linked to” or “operatively linked to” or “operably associated with” the regulatory elements, meaning that the gene or coding region is controlled or influenced by the regulatory element. For instance, a promoter is operably linked to a coding sequence if the promoter effects transcription or expression of the coding sequence.

### Exported RNA Reporters for Live-Cell Measurement

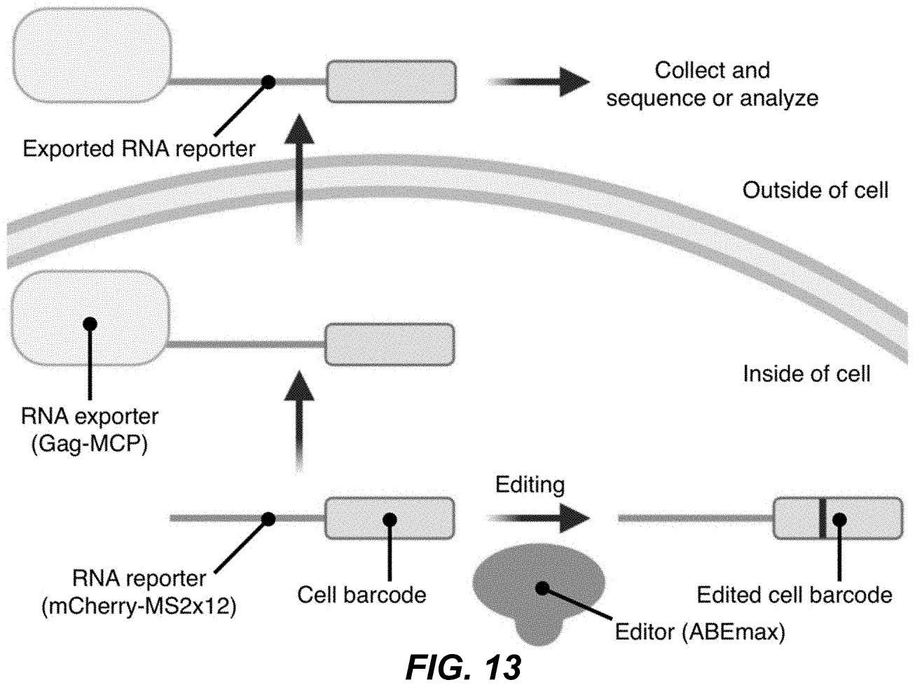

Provided herein include compositions, methods, systems, and kits for measurement of the states of living cells across time. Provided herein are live single-cell reporter systems that address the above-mentioned challenges in the art. In some embodiments, the system is based on the export and sequencing of cellular RNA using minimally perturbative engineered components and a barcoding strategy that enables single-cell resolution (See e.g., FIG. 13). The system can be composed of an RNA exporter and exported reporter RNA, which encodes information in its sequence about the state of the cell. The system can further include a barcode sequence that is associated with the reporter RNA, either directly encoded within the sequence of the reporter RNA itself, or bound to the reporter RNA. This barcode can uniquely identify the single cell from which the RNA originated. Collecting and sequencing or otherwise analyzing the reporter RNA can then yield information about the state of a cell. Further, in some embodiments, samples of exported RNA can be collected longitudinally (over time), enabling measurement of single-cell state dynamics via temporally-resolved readouts of molecules directly produced by the individual cells being analyzed. This type of clonal or single-cell longitudinal tracking is not possible in most tissue contexts using existing tools.

Systems provided herein can be implemented using an RNA exporter, which can be a capsid protein of viral origin, such as retroviral Gag protein, fused with an RNA binding protein, such as MS2 coat protein (See e.g., FIG. 13). The exported reporter RNA (cargo) can consist of an mRNA, such as that encoding the fluorescent protein mCherry, tagged with cognate RNA aptamers, such as MS2 stem-loops. The reporter RNA sequence can contain barcodes that are unique to individual cells, such as random nucleotide sequences introduced using high-diversity libraries. The reporter RNA can further contain an editable barcode region that can be altered (edited) by an editor, such as a base editor, to enable reconstruction of lineage relationships among distinct cells and to ensure that the barcode uniquely identifies an individual cell despite clonal inheritance during cell division. These components can be stably integrated into the genome of a mammalian cell to enable long-term reporting.

The methods, compositions, and systems provided herein encompass a number of different embodiments and applications, and include the following:

Engineered RNA Exporters

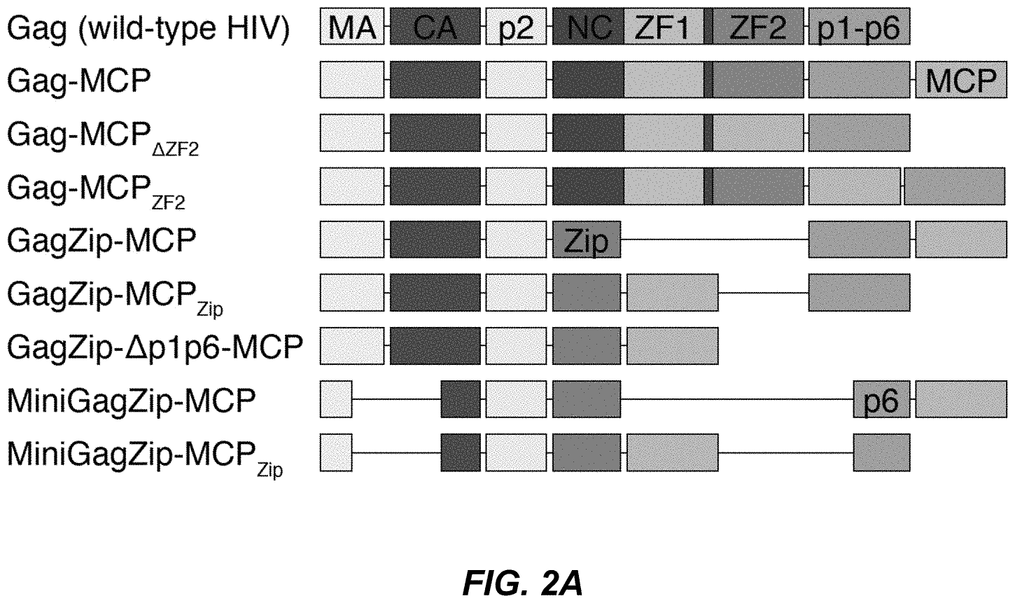

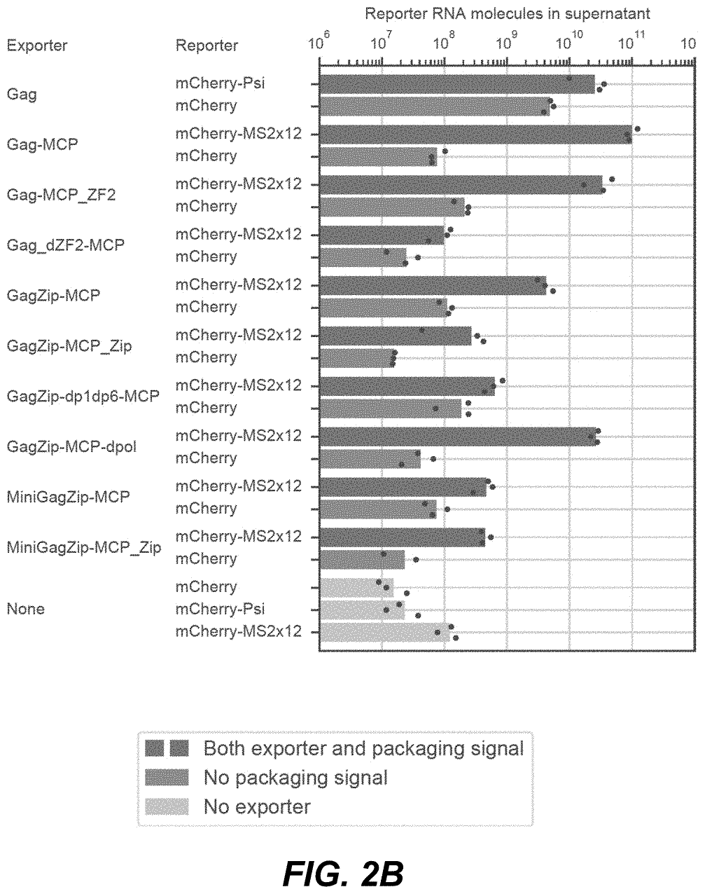

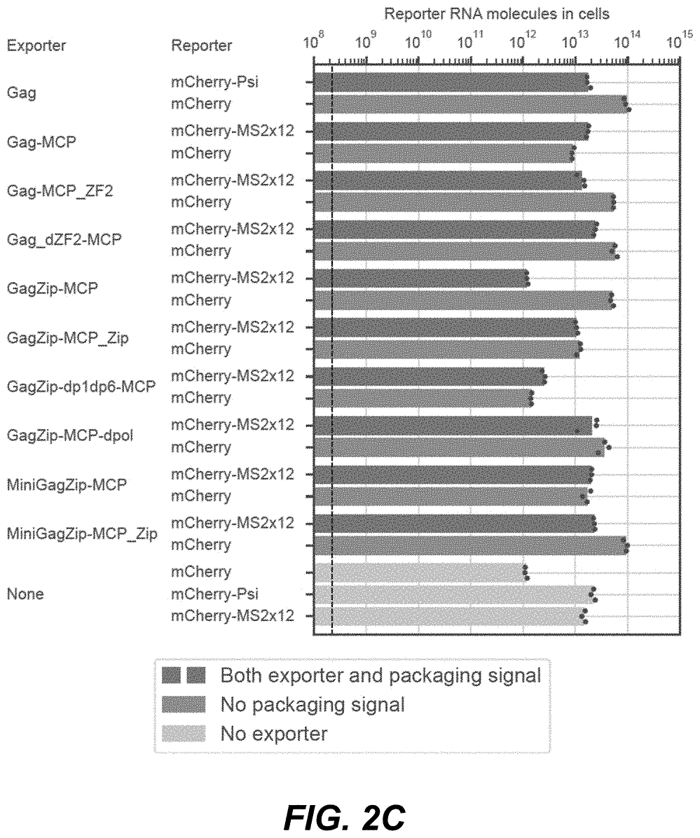

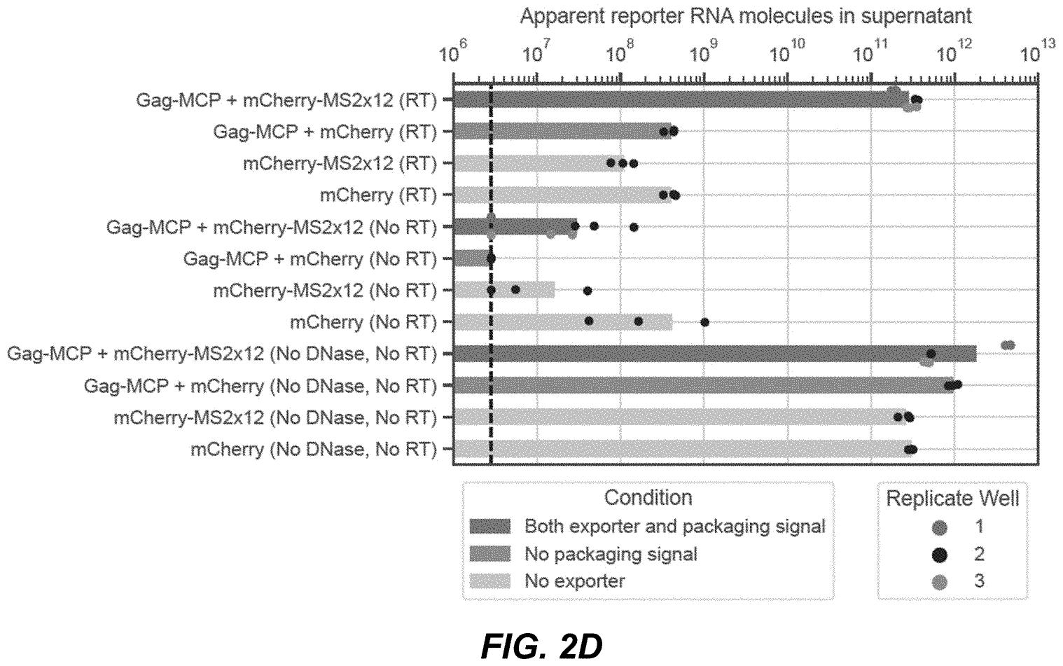

In some embodiments provided herein RNA exporters are further engineered to be minimally perturbative to cellular physiology or minimally immunogenic. RNA exporters can be engineered from components originating from viruses, including endogenous and commensal viruses, or non-viral compartmentalization and secretion systems (See e.g., FIG. 2).

De Novo Designed RNA Exporters

RNA export systems of viral origin are adapted for viral replication and therefore, in some embodiments, can suffer from the drawback that their components may perturb cellular function and be recognized by immune systems. Provided herein include RNA exporters based on de novo designed proteins, such as enveloped protein nanocages (Votteler et al. 2016), which can be designed or engineered to minimize perturbation or immunogenicity.

RNA Binding Domains

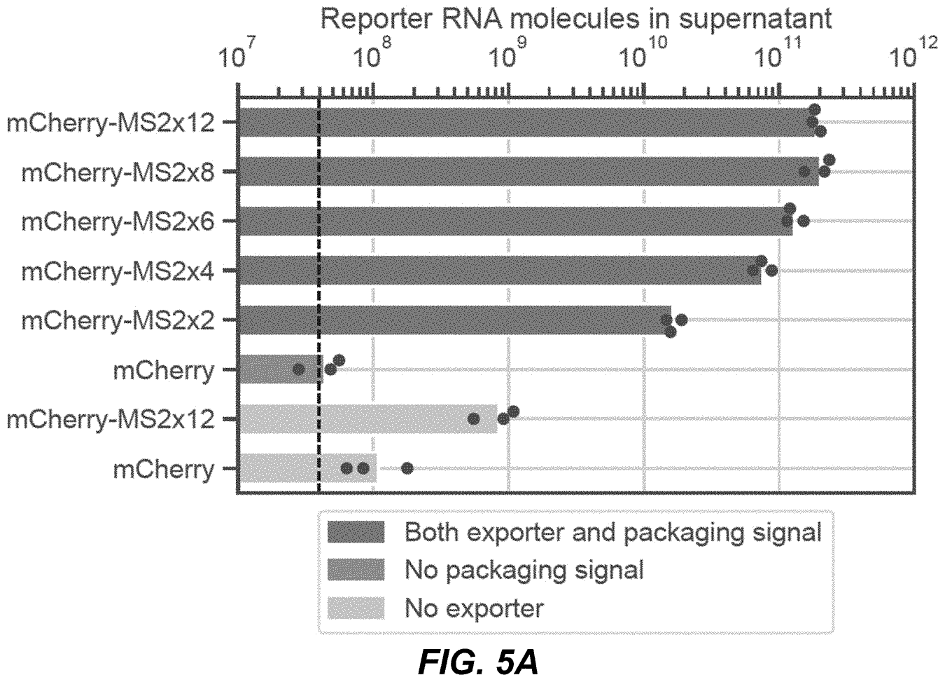

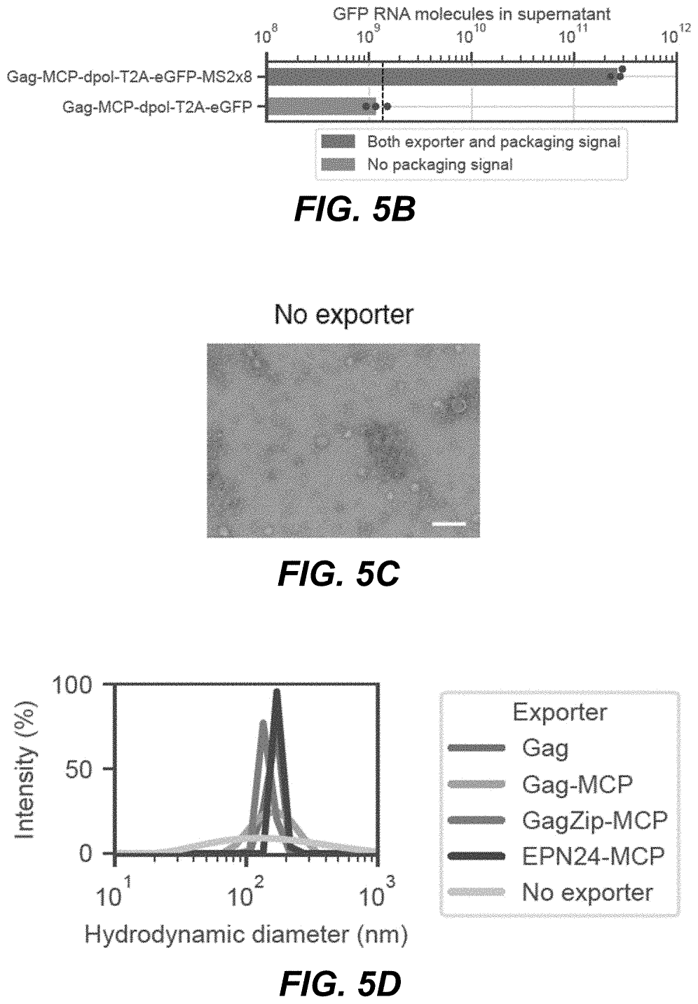

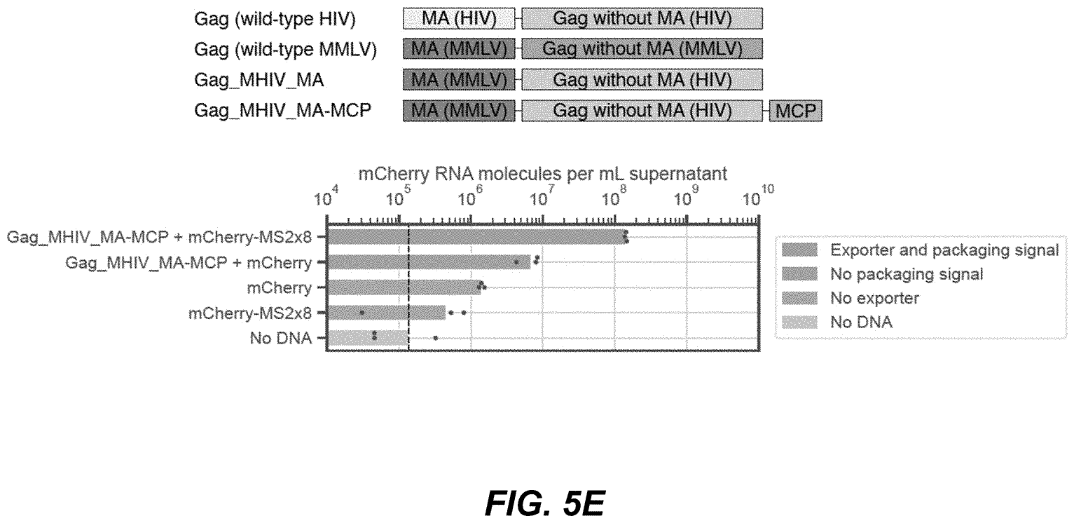

To enable specific and programmable export of RNA molecules, some embodiments of the methods, compositions, and systems provided herein include the use of RNA aptamers and cognate RNA binding domains, as well as fusion of such domains to compartmentalization and secretion components, as described above. These RNA binding systems can include natural or engineered viral packaging signals, the MS2/MS2 coat protein system, the PP7/PP7 coat protein system, catalytically inactivated CRISPR-Cas9 or -Cas13, Pumilio, and nonsequence-specific RNA-binding proteins (See e.g., FIG. 2, FIG. 5). RNA can also be bound in a sequence-specific manner by hybridization to an RNA probe.

Evolving Barcodes

In some embodiments, achieving single-cell resolution of live-cell RNA reporters requires barcodes that are unique to each individual cell. To enable unique single-cell barcoding that is robust to cell division and clonal expansion, some embodiments of the methods, compositions, and systems provided herein can include the use of DNA editors that dynamically and stochastically alter (edit) a cell's barcode, such that sister cells, which initially share the same cell barcode by inheritance from a common progenitor cell at cell division, acquire distinct and unique barcode sequences. These editors can include CRISPR-Cas9, base editors, prime editors, integrases, and recombinases.

Monitoring Cell Population Dynamics and Reconstructing Lineage

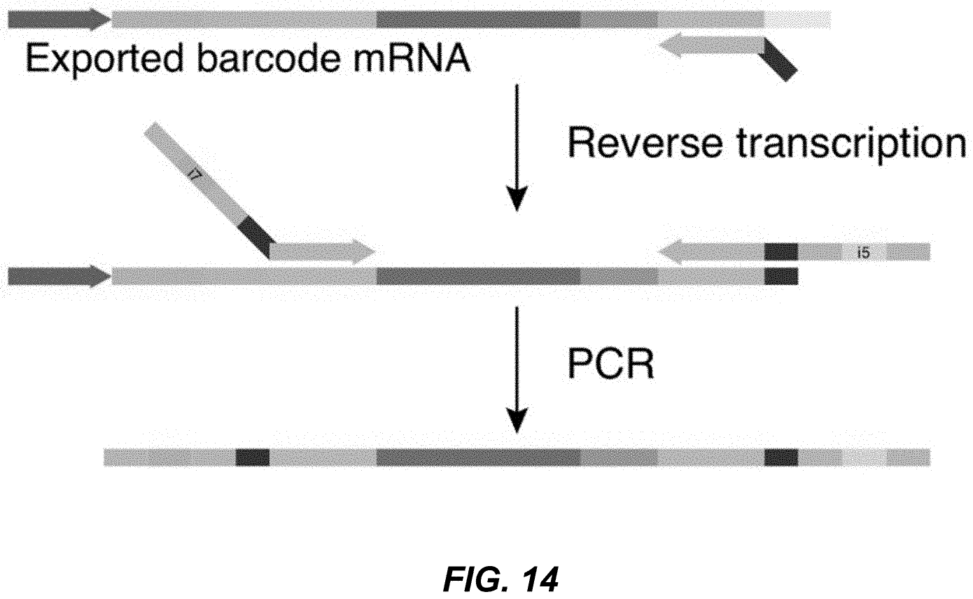

Cells form populations through cell division and death, giving rise to clonal population structure. Existing methods for measuring population structure require destructive sampling of cells, only detect cells that survive to the endpoint of measurement, or do not permit direct calibration to real time. The methods, compositions, and systems provided herein can enable non-destructive real-time monitoring of cell population dynamics by longitudinal sampling and sequencing of static clone barcodes and evolving lineage barcodes (See e.g., FIG. 14). These dynamics can be used to identify genetic or molecular perturbations which affect cellular growth, with applications in cancer, developmental disorders, and other diseases. The systems, methods, compositions, and kits provided herein can, in some embodiments, be employed in concert with the systems, methods, compositions, and kits described in U.S. Provisional Patent Application No. 63/396,537, entitled, “Regenerative Editing For Molecular Recording,” filed Aug. 9, 2022, the content of which is incorporated herein by reference in its entirety.

Transcriptional Reporters of Cell State

The methods, compositions, and systems provided herein can include RNA reporters that are driven in a cell state-responsive manner, such that the level of the transcript or the relative levels of different transcripts serves as an indicator of cell state. Examples include regulation by transcription factor-dependent, signal-responsive, metabolic, or circadian promoters. Many such reporters can be multiplexed by assigning them distinct sequence barcodes. Recovering longitudinal dynamics of different organs, or individual cells within specific organs, non-invasively from a single animal is difficult with other approaches.

Live-Cell Transcriptome Reporters

Single-cell transcriptomes can be used to determine cell type and state. Existing methods for measuring single-cell transcriptomes require destruction of the cell and only yield a snapshot of information from one timepoint. The methods, compositions, and systems provided herein can include systems to export samples of the transcriptome from cells in a non-destructive manner, enabling continuous measurement of transcriptome dynamics of single cells. Transcripts originating from the same cell can share the same barcode, enabling identification of multiple transcripts from each cell. These transcripts can be captured by RNA binding proteins, aptamers, or hybridization with RNA probes. The barcode can be physically linked to each transcript by ligation, polymerization, primer extension, or physical co-compartmentalization.

Disclosed herein include compositions. In some embodiments, the composition is or comprises a nucleic acid composition. The nucleic acid composition can comprise: one or more first polynucleotide(s) encoding an RNA exporter protein; and/or one or more second polynucleotide(s) each encoding one or more reporter RNA molecule(s). In some embodiments, the RNA exporter protein comprises: an RNA-binding domain, a membrane-binding domain, and an interaction domain capable of nucleating self-assembly. In some embodiments, a plurality of RNA exporter proteins are capable of self-assembling into lipid-enveloped nanoparticles (LNs) secreted from a reporter cell in which the RNA exporter proteins are expressed, thereby generating a population of LNs comprising exported reporter RNA molecule(s).

Disclosed herein include compositions. In some embodiments, the composition is or comprises a population of reporter cells. In some embodiments, the composition comprises: a population of reporter cells comprising a nucleic acid composition disclosed herein. The population of reporter cells can comprise: one or more first polynucleotide(s) encoding an RNA exporter protein and/or one or more second polynucleotide(s) each encoding one or more reporter RNA molecule(s). In some embodiments, the RNA exporter protein comprises: an RNA-binding domain, a membrane-binding domain, and an interaction domain capable of nucleating self-assembly. In some embodiments, a plurality of RNA exporter proteins are capable of self-assembling into lipid-enveloped nanoparticles (LNs) secreted from a reporter cell in which the RNA exporter proteins are expressed, thereby generating a population of LNs comprising exported reporter RNA molecule(s).

The reporter RNA molecule(s) can comprise one or more barcode(s). The one or more barcodes can comprise reporter barcode(s) and/or cell barcode(s). The reporter RNA molecule(s) each can comprise packing signal(s), one or more cell barcode(s), and a reporter barcode. The composition can comprise: one or more third polynucleotide(s) each encoding one or more packing RNA molecule(s), wherein the LNs further comprise exported packing RNA molecule(s), wherein the packing RNA molecule(s) comprise a capture domain, and wherein the reporter RNA molecule(s) comprise a hybridization domain capable of hybridizing to the capture domain. In some embodiments, the reporter RNA molecule(s) each comprise a reporter barcode and one or more cell barcodes; and the packing RNA molecule(s) each comprise packing signal(s). In some embodiments, the reporter RNA molecule(s) each comprise a reporter barcode; and the packing RNA molecule(s) each comprise packing signal(s) and one or more cell barcode(s). In some embodiments, the reporter RNA molecule(s) each comprise packing signal(s) and a reporter barcode; and the packing RNA molecule(s) each comprise one or more cell barcode(s).

In some embodiments, the cell barcode(s) comprise: a clone barcode. Each reporter cell of a population of reporter cells can have a single clone barcode. The sequence of clone barcode can be unique to each reporter cell of the population of reporter cells at an initial time point. Progeny cells arising from cell division of the same reporter cell can constitute a clonal population wherein each clone comprises the same clone barcode. The clone barcode can be static. The clone barcode can be selected from a library of at least about 1000, 1100, 1200, 1300, 1400, 1500, 1600, 1700, 1800, 1900, 2000, 2100, 2200, 2300, 2400, 2500, 2600, 2700, 2800, 2900, 3000, 3250, 3500, 3750, 4000, 4250, 4500, 4750, 5000, 5500, 6000, 6500, 7000, 7500, 8000, 8500, 9000, 9500, 10000, 15000, 20000, 25000, or 30000 different clone barcode sequences.

In some embodiments, the cell barcode(s) comprise: a subpopulation barcode. A population of reporter cells can comprise one or more reporter cell subpopulations. Each reporter cell subpopulation can have a single subpopulation barcode. The sequence of the subpopulation barcode can be unique to each reporter cell subpopulation of the population of reporter cells at an initial time point. Progeny cells arising from cell division of the same reporter cell can share the same subpopulation barcode. The subpopulation barcode can be static. The subpopulation barcode can be selected from a library of at least about 1000, 1100, 1200, 1300, 1400, 1500, 1600, 1700, 1800, 1900, 2000, 2100, 2200, 2300, 2400, 2500, 2600, 2700, 2800, 2900, 3000, 3250, 3500, 3750, 4000, 4250, 4500, 4750, 5000, 5500, 6000, 6500, 7000, 7500, 8000, 8500, 9000, 9500, 10000, 15000, 20000, 25000, or 30000 different subpopulation barcode sequences.

In some embodiments, the cell barcode(s) comprise: a lineage barcode. Each reporter cell of a population of reporter cells can have a single lineage barcode. In some embodiments, the lineage barcode is not static. The lineage barcode can be an editable barcode. At least about 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 75%, 100%, or a number or a range between any two of these values, of progeny cells arising from cell division of a reporter cell can have a lineage barcode different than progeny cells arising from cell division of the same reporter cell. The sequence of the lineage barcode can be unique to each reporter cell of the population of reporter cells at an initial time point.

The clone barcode, the subpopulation barcode, and/or the lineage barcode can be from about 4 nucleotides to about 30 nucleotides in length. The packing RNA molecule(s) and/or reporter RNA molecule(s) can be mRNA. The barcode(s) and/or packing signal(s) can be situated in the 5′UTR and/or 3′UTR. The reporter RNA molecule(s) and/or packing RNA molecule(s) can be transcribed from a RNA polymerase I promoter, a RNA polymerase II promoter, a RNA polymerase III promoter, a T7 promoter, a T3 promoter, or any combination thereof.

The composition can comprise: one or more fourth polynucleotide(s) encoding an editor and/or a targeting molecule. The targeting molecule can comprise single strand DNA or single strand RNA. The targeting molecule can comprise a guide RNA (gRNA). The editor can be selected from the group comprising CRISPR-Cas9, base editors, prime editors, integrases, and recombinases. The editor can be a base editor capable of base editing the lineage barcode. In some embodiments, said base editing comprises: adenine (A)-to-guanine (G) base editing and/or cytosine (C)- to-thymine (T) base editing. The base editor can comprise saCas9-KKH, Cas9-VQR, Cas9-VRQR, Cas9-VRER, Cas9-NG, ABE7.7, pNMG-624, ABE3.2, ABE5.3, pNMG-558, pNMG-576, pNMG-577, pNMG-586, ABE7.2, pNMG-620, pNMG-617, pNMG-618, pNMG-620, pNMG-621, pNGM-622, pNMG-623, ABE6.3, ABE6.4, ABE7.8, ABE7.9, ABE7.10, ABEMax, ABE8e, CP1028-ABE8e, ABE7.10-CP1041, CP1041-ABE8e, or any combination thereof. The base editor can comprise an adenine base editor (ABE) and/or a cytosine base editor (CBE). The ABE can comprise monomer and dimer versions of one or more of ABE8e, ABE8e-V106W, SaABE8e, SaKKH-ABE8e, NG-ABE8e, ABE-xCas9, ABE8e-NRTH, ABE8e-NRRH, ABE8e-NRCH, ABE8e-NG-CP1041, ABE8e-VRQR-CP1041, ABE8e-CP1041, ABE8e-CP1028, ABE8e-VRQR, ABE8e-LbCas12a (LbABE8e), ABE8e-AsCas12a (enAsABE8e), ABE8e-SpyMac, ABE8e (TadA-8e V106W), ABE8e (K20A,R21A), and ABE8e(TadA-8e V82G).

The lineage barcode can comprise about 2 to about 40 (e.g., 2, 3, 4, 5, 6, 7, 8, 9, 10, 11, 12, 13, 14, 15, 16, 17, 18, 19, 20, 25, 30, 35, 40, or a number or a range between any two of these values) editable units. The editable units can have the same length and/or sequence. The lineage barcode can comprise from about 2 tandem copies to about 40 tandem copies (e.g., 2, 3, 4, 5, 6, 7, 8, 9, 10, 11, 12, 13, 14, 15, 16, 17, 18, 19, 20, 25, 30, 35, 40, ora number ora range between any two of these values) of an editable unit. In some embodiments, an editable unit can be about 2 nucleotides to about 40 (e.g., 2, 3, 4, 5, 6, 7, 8, 9, 10, 11, 12, 13, 14, 15, 16, 17, 18, 19, 20, 25, 30, 35, 40, or a number or a range between any two of these values) nucleotides in length. In some embodiments, an editable unit can comprise at least 2, 3, 4, 5, 6, 7, 8, 9, or 10, bases that can be converted from A to G or from C to T by the base editor, wherein the fraction of editable bases converted from A to G or from C to T by the base editor increases through time. The lineage barcode can comprise one or more gRNA targeting sequences, and a gRNA can be capable of targeting gRNA targeting sequence(s). The gRNA targeting sequence can be about 13, 14, 15, 16, 17, 18, 19, 20, 21, 22, 23, 24, or 25, nucleotides in length. In some embodiments, each editable unit can comprise a gRNA targeting sequence. In some embodiments, each editable unit can comprise a Protospacer Adjacent Motif (PAM). The PAM can be downstream of the gRNA targeting sequence. In some embodiments, said base editing can comprise base editing the lineage barcode in one reporter cell, at least one reporter cell, or each of a population of reporter cells at one or more predetermined time points. In some embodiments, said base editing can comprise base editing the lineage barcode in one reporter cell, at least one reporter cell, or each of a population of reporter cells at an edit rate. The edit rate can be predetermined. The edit rate can be about 1% to about 100% (e.g., 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, or a number or a range between any two of these values) edit per unit time. The edit rate can be about 1% to 100% (e.g., 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, or a number or a range between any two of these values) edit per cell per cell division cycle.

In some embodiments, the RNA exporter protein is a chimeric fusion protein or a multi-subunit protein. The RNA exporter protein can comprise two or more components configured to dimerize via dimerization domain(s). The RNA binding domain can be capable of binding the packing signal(s), and the packing RNA molecule(s) and/or reporter RNA molecule(s) can be specifically packaged into the LNs via interaction of the packing signal(s) with the RNA-binding domain of the RNA exporter protein. The abundance of reporter RNA molecule(s) exported to the exterior of a reporter cell can be at least about 1.1-fold (e.g., 0.1-fold, 1.2-fold, 1.3-fold, 1.4-fold, 1.5-fold, 1.6-fold, 1.7-fold, 1.8-fold, 1.8-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) higher as compared to a reporter cell wherein (i) the packing signal(s) are absent from the reporter RNA molecule(s) and/or packing RNA molecule(s) and/or (ii) the RNA exporter protein does not comprise an RNA binding domain. The packing signal(s) can comprise an array of at least about 2, 3, 4, 5, 6, 7, 8, 9, 10, 11, 12, 13, or 14, tandem repeats of an aptamer (e.g., a MS2 stem-loop aptamer). The rate of export of packing RNA molecule(s) and/or reporter RNA molecule(s) can be capable of being configured by varying the number of tandem repeats.

The RNA exporter protein can be configured to be minimally perturbative to cellular physiology and/or minimally immunogenic. The RNA exporter protein can comprise or can be derived from one or more components of viral origin, one or more components of a non-viral compartmentalization and secretion system, and/or one or more components of de novo designed proteins. The RNA exporter protein can comprise a capsid protein of viral origin, optionally fused with an RNA binding protein. The one or more first polynucleotide(s) encoding the RNA exporter protein can comprise packing signal(s), and LNs thereby can comprise RNA molecules encoding the RNA exporter protein. The RNA binding domain can comprise or can be derived from an RNA binding protein. The packing signal(s) can comprise a Ku binding hairpin and the RNA binding protein can be Ku. The packing signal(s) can comprise a telomerase Sm7 binding motif and the RNA binding protein can be Sm7. The packing signal(s) can comprise an MS2 phage operator stem-loop and the RNA binding protein can be MS2 Coat Protein (MCP). The packing signal(s) can comprise a PP7 phage operator stem-loop and the RNA binding protein can be PP7 Coat Protein (PCP). The packing signal(s) can comprise an SfMu phage Com stem-loop and the RNA binding protein can be Com RNA binding protein. The packing signal(s) can comprise a PUF binding site (PBS) and the RNA binding protein can be Pumilio/fem-3 mRNA binding factor (PUF). The packing signal(s) can comprise an MMLV packing signal (Psi) and the RNA binding protein can be MMLV.

The RNA binding domain can comprise or can be derived from a catalytically inactivated programmable nuclease configured to bind the packing signal(s) (e.g., catalytically inactivated CRISPR-Cas9 or -Cas13). The RNA exporter protein can comprise at least a portion of a viral capsid protein (e.g., a retroviral Gag protein). At least a portion of the nucleocapsid domain, matrix domain, a zinc finger domain, a p1 domain, capsid domain and/or a p1-p6 domain can be removed from said viral capsid protein. The nucleocapsid domain can be replaced with a leucine zipper (e.g., the leucine zipper of GCN4). The interaction domain can comprise or can be derived from a viral capsid protein (e.g., a retroviral Gag protein). The interaction domain can comprise dimerization domain(s), e.g., leucine zipper dimerization domain from GCN4 (Zip). The dimerization domain(s) can comprise or can be derived from SYNZIP1, SYNZIP2, SYNZIP3, SYNZIP4, SYNZIP5, SYNZIP6, SYNZIP7, SYNZIP8, SYNZIP9, SYNZIP10, SYNZIP11, SYNZIP12, SYNZIP13, SYNZIP14, SYNZIP15, SYNZIP16, SYNZIP17, SYNZIP18, SYNZIP19, SYNZIP20, SYNZIP21, SYNZIP22, SYNZIP23, BATF, FOS, ATF4, BACH1, JUND, NFE2L3, AZip, BZip, a PDZ domain ligand, an SH3 domain, a PDZ domain, a GTPase binding domain, a leucine zipper domain, an SH2 domain, a PTB domain, an FHA domain, a WW domain, a 14-3-3 domain, a death domain, a caspase recruitment domain, a bromodomain, a chromatin organization modifier, a shadow chromo domain, an F-box domain, a HECT domain, a RING finger domain, a sterile alpha motif domain, a glycine-tyrosine-phenylalanine domain, a SNAP domain, a VHS domain, an ANK repeat, an armadillo repeat, a WD40 repeat, an MH2 domain, a calponin homology domain, a Dbl homology domain, a gelsolin homology domain, a PB1 domain, a SOCS box, an RGS domain, a Toll/IL-1 receptor domain, a tetratricopeptide repeat, a TRAF domain, a Bcl-2 homology domain, a coiled-coil domain, a bZIP domain, portions thereof, variants thereof, or any combination thereof. The dimerization domain(s) can be a homodimerization domain or a multimerization domain (e.g., a homo- or hetero-dimerizing or multimerizing leucine zipper, a PDZ domains, a SH3 domain, aGBD domain, or any combination thereof).

The RNA exporter protein can self-assemble to form nanocages, and the LNs can comprise a plurality of said nanocages. The interaction domain can comprise 13-01 and/or the membrane binding domain can comprise rat phospholipase C delta Pleckstrin Homology domain. The RNA exporter protein can comprise enveloped protein nanocage domain EPN24 fused with an RNA binding protein. In some embodiments, the RNA exporter protein comprises: a myristoylation motif of HIV-1 NL4-3 Gag, optionally amino acid residues 2-6; a myristoylation/palmitoylation motif of Lyn kinase, optionally amino acid residues 2-13; a Pleckstrin Homology domain of rat phospholipase Cδ; and/or a p6 domain of HIV-1 NL4-3 Gag. The RNA exporter protein can be configured to operate in a cell type and/or species different than the cell type and/or species in which at least one parental component of said RNA exporter protein operates. The RNA exporter protein can be a species-chimeric RNA exporter protein. The RNA exporter protein can comprise a chimeric viral capsid protein wherein the matrix domain is replaced with the matrix domain of another virus. The RNA exporter protein can comprise an amino acid sequence at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values, identical to any one of SEQ ID NOS: 1-24 (Table 1). In some embodiments, the expression of the RNA exporter protein in the reporter cell(s) does not alter the endogenous transcriptome, morphology, and/or physiology of said reporter cell(s). The RNA exporter protein can be constitutively expressed by the reporter cell(s). The RNA exporter protein can be configured to exhibit minimal immunogenicity in a subject, e.g., derived from commensal viruses or endogenous viruses, and/or novo designed proteins and/or humanized. Table 2 shows exemplary implementations of RNA exporters and exported RNA reporters.

TABLE 1
AMINO ACID SEQUENCES OF RNA EXPORTER PROTEINS

| | SEQ | |
|---|---|---|
| Name | ID NO | Sequence |
| EPN1 | 1 | MGARASGSKSGSGSDSGSKIEELFKKHKIVAVLRANSVEEAKKKALAVFLGG |
| | | VHLIEITFTVPDADTVIKELSFLKEMGAIIGAGTVTSVEQCRKAVESGAEFI |
| | | VSPHLDEEISQFCKEKGVFYMPGVMTPTELVKAMKLGHTILKLFPGEVVGPQ |
| | | FVKAMKGPFPNVKFVPTGGVNLDNVCEWFKAGVLAVGVGSALVKGTPVEVAE |
| | | KAKAFVEKIRGCTEQKLISEEDLQSRPEPTAPPEESFRSGVETTTPPQKQEP |
| | | IDKELYPLTSLRSLFGNDPSSQ |
| EPN1-MCP | 2 | MGARASGSKSGSGSDSGSKIEELFKKHKIVAVLRANSVEEAKKKALAVFLGG |
| | | VHLIEITFTVPDADTVIKELSFLKEMGAIIGAGTVTSVEQCRKAVESGAEFI |
| | | VSPHLDEEISQFCKEKGVFYMPGVMTPTELVKAMKLGHTILKLFPGEVVGPQ |
| | | FVKAMKGPFPNVKFVPTGGVNLDNVCEWFKAGVLAVGVGSALVKGTPVEVAE |
| | | KAKAFVEKIRGCTEQKLISEEDLQSRPEPTAPPEESFRSGVETTTPPQKQEP |
| | | IDKELYPLTSLRSLFGNDPSSQASNFTQFVLVDNGGTGDVTVAPSNFANGVA |
| | | EWISSNSRSQAYKVTCSVRQSSAQKRKYTIKVEVPKVATQTVGGVELPVAAW |
| | | RSYLNMELTIPIFATNSDCELIVKAMQGLLKDGNPIPSAIAANSGIY |
| EPN1-MCP_I | 3 | MGARASGSKSGSGSDSGSKIEELFKKHKIVAVLRANSVEEAKKKALAVFLGG |
| | | VHLIEITFTVPDADTVIKELSFLKEMGAIIGAGTVTSVEQCRKAVESGAEFI |
| | | VSPHLDEEISQFCKEKGVFYMPGVMTPTELVKAMKLGHTILKLFPGEVVGPQ |
| | | FVKAMKGPFPNVKFVPTGGVNLDNVCEWFKAGVLAVGVGSALVKGTPVEVAE |
| | | KAKAFVEKIRGCTASNFTQFVLVDNGGTGDVTVAPSNFANGVAEWISSNSRS |
| | | QAYKVTCSVRQSSAQKRKYTIKVEVPKVATQTVGGVELPVAAWRSYLNMELT |
| | | IPIFATNSDCELIVKAMQGLLKDGNPIPSAIAANSGIYEQKLISEEDLQSRP |
| | | EPTAPPEESFRSGVETTTPPQKQEPIDKELYPLTSLRSLFGNDPSSQ |
| EPN1-MCP_Myc | 4 | MGARASGSKSGSGSDSGSKIEELFKKHKIVAVLRANSVEEAKKKALAVFLGG |
| | | VHLIEITFTVPDADTVIKELSFLKEMGAIIGAGTVTSVEQCRKAVESGAEFI |
| | | VSPHLDEEISQFCKEKGVFYMPGVMTPTELVKAMKLGHTILKLFPGEVVGPQ |
| | | FVKAMKGPFPNVKFVPTGGVNLDNVCEWFKAGVLAVGVGSALVKGTPVEVAE |
| | | KAKAFVEKIRGCTEQKLISEEDLASNFTQFVLVDNGGTGDVTVAPSNFANGV |
| | | AEWISSNSRSQAYKVTCSVRQSSAQKRKYTIKVEVPKVATQTVGGVELPVAA |
| | | WRSYLNMELTIPIFATNSDCELIVKAMQGLLKDGNPIPSAIAANSGIYLQSR |
| | | PEPTAPPEESFRSGVETTTPPQKQEPIDKELYPLTSLRSLFGNDPSSQ |
| EPN11 | 5 | MGCIKSKRKDNLNLQSRPEPTAPPEESFRSGVETTTPPQKQEPIDKELYPLT |
| | | SLRSLFGNDPSSQKIEELFKKHKIVAVLRANSVEEAKKKALAVFLGGVHLIE |
| | | ITFTVPDADTVIKELSFLKEMGAIIGAGTVTSVEQCRKAVESGAEFIVSPHL |
| | | DEEISQFCKEKGVFYMPGVMTPTELVKAMKLGHTILKLFPGEVVGPQFVKAM |
| | | KGPFPNVKFVPTGGVNLDNVCEWFKAGVLAVGVGSALVKGTPVEVAEKAKAF |
| | | VEKIRGCTEQKLISEEDL |
| EPN11-MCP | 6 | MGCIKSKRKDNLNLQSRPEPTAPPEESFRSGVETTTPPQKQEPIDKELYPLT |
| | | SLRSLFGNDPSSQKIEELFKKHKIVAVLRANSVEEAKKKALAVFLGGVHLIE |
| | | ITFTVPDADTVIKELSFLKEMGAIIGAGTVTSVEQCRKAVESGAEFIVSPHL |
| | | DEEISQFCKEKGVFYMPGVMTPTELVKAMKLGHTILKLFPGEVVGPQFVKAM |
| | | KGPFPNVKFVPTGGVNLDNVCEWFKAGVLAVGVGSALVKGTPVEVAEKAKAF |
| | | VEKIRGCTEQKLISEEDLASNFTQFVLVDNGGTGDVTVAPSNFANGVAEWIS |
| | | SNSRSQAYKVTCSVRQSSAQKRKYTIKVEVPKVATQTVGGVELPVAAWRSYL |
| | | NMELTIPIFATNSDCELIVKAMQGLLKDGNPIPSAIAANSGIY |
| EPN11-MCP_I | 7 | MGCIKSKRKDNLNLQSRPEPTAPPEESFRSGVETTTPPQKQEPIDKELYPLT |
| | | SLRSLFGNDPSSQKIEELFKKHKIVAVLRANSVEEAKKKALAVFLGGVHLIE |
| | | ITFTVPDADTVIKELSFLKEMGAIIGAGTVTSVEQCRKAVESGAEFIVSPHL |
| | | DEEISQFCKEKGVFYMPGVMTPTELVKAMKLGHTILKLFPGEVVGPQFVKAM |
| | | KGPFPNVKFVPTGGVNLDNVCEWFKAGVLAVGVGSALVKGTPVEVAEKAKAF |
| | | VEKIRGCTASNFTQFVLVDNGGTGDVTVAPSNFANGVAEWISSNSRSQAYKV |
| | | TCSVRQSSAQKRKYTIKVEVPKVATQTVGGVELPVAAWRSYLNMELTIPIFA |
| | | TNSDCELIVKAMQGLLKDGNPIPSAIAANSGIYEQKLISEEDL |
| EPNII-MCP_p6 | 8 | MGCIKSKRKDNLNLQSRPEPTAPPEESFRSGVETTTPPQKQEPIDKELYPLT |
| | | SLRSLFGNDPSSQASNFTQFVLVDNGGTGDVTVAPSNFANGVAEWISSNSRS |
| | | QAYKVTCSVRQSSAQKRKYTIKVEVPKVATQTVGGVELPVAAWRSYLNMELT |
| | | IPIFATNSDCELIVKAMQGLLKDGNPIPSAIAANSGIYKIEELFKKHKIVAV |
| | | LRANSVEEAKKKALAVFLGGVHLIEITFTVPDADTVIKELSFLKEMGAIIGA |
| | | GTVTSVEQCRKAVESGAEFIVSPHLDEEISQFCKEKGVFYMPGVMTPTELVK |
| | | AMKLGHTILKLFPGEVVGPQFVKAMKGPFPNVKFVPTGGVNLDNVCEWFKAG |
| | | VLAVGVGSALVKGTPVEVAEKAKAFVEKIRGCTEQKLISEEDL |
| EPN24 | 9 | MHGLQDDPDLQALLKGSQLLKVKSSSWRRERFYKLQEDCKTIWQESRKVMRS |
| | | PESQLFSIEDIQEVRMGHRTEGLEKFARDIPEDRCFSIVFKDQRNTLDLIAP |
| | | SPADAQHWVQGLRKIIHHSGSMDQRQKLQSRPEPTAPPEESFRSGVETTTPP |
| | | QKQEPIDKELYPLTSLRSLFGNDPSSQKIEELFKKHKIVAVLRANSVEEAKK |
| | | KALAVFLGGVHLIEITFTVPDADTVIKELSFLKEMGAIIGAGTVTSVEQCRK |
| | | AVESGAEFIVSPHLDEEISQFCKEKGVFYMPGVMTPTELVKAMKLGHTILKL |
| | | FPGEVVGPQFVKAMKGPFPNVKFVPTGGVNLDNVCEWFKAGVLAVGVGSALV |
| | | KGTPVEVAEKAKAFVEKIRGCTEQKLISEEDL |
| EPN24-MCP | 10 | MHGLQDDPDLQALLKGSQLLKVKSSSWRRERFYKLQEDCKTIWQESRKVMRS |
| | | PESQLFSIEDIQEVRMGHRTEGLEKFARDIPEDRCFSIVFKDQRNTLDLIAP |
| | | SPADAQHWVQGLRKIIHHSGSMDQRQKLQSRPEPTAPPEESFRSGVETTTPP |
| | | QKQEPIDKELYPLTSLRSLFGNDPSSQKIEELFKKHKIVAVLRANSVEEAKK |
| | | KALAVFLGGVHLIEITFTVPDADTVIKELSFLKEMGAIIGAGTVTSVEQCRK |
| | | AVESGAEFIVSPHLDEEISQFCKEKGVFYMPGVMTPTELVKAMKLGHTILKL |
| | | FPGEVVGPQFVKAMKGPFPNVKFVPTGGVNLDNVCEWFKAGVLAVGVGSALV |
| | | KGTPVEVAEKAKAFVEKIRGCTEQKLISEEDLASNFTQFVLVDNGGTGDVTV |
| | | APSNFANGVAEWISSNSRSQAYKVTCSVRQSSAQKRKYTIKVEVPKVATQTV |
| | | GGVELPVAAWRSYLNMELTIPIFATNSDCELIVKAMQGLLKDGNPIPSAIAA |
| | | NSGIY |
| EPN24-MCP_I | 11 | MHGLQDDPDLQALLKGSQLLKVKSSSWRRERFYKLQEDCKTIWQESRKVMRS |
| | | PESQLFSIEDIQEVRMGHRTEGLEKFARDIPEDRCFSIVFKDQRNTLDLIAP |
| | | SPADAQHWVQGLRKIIHHSGSMDQRQKLQSRPEPTAPPEESFRSGVETTTPP |
| | | QKQEPIDKELYPLTSLRSLFGNDPSSQKIEELFKKHKIVAVLRANSVEEAKK |
| | | KALAVFLGGVHLIEITFTVPDADTVIKELSFLKEMGAIIGAGTVTSVEQCRK |
| | | AVESGAEFIVSPHLDEEISQFCKEKGVFYMPGVMTPTELVKAMKLGHTILKL |
| | | FPGEVVGPQFVKAMKGPFPNVKFVPTGGVNLDNVCEWFKAGVLAVGVGSALV |
| | | KGTPVEVAEKAKAFVEKIRGCTASNFTQFVLVDNGGTGDVTVAPSNFANGVA |
| | | EWISSNSRSQAYKVTCSVRQSSAQKRKYTIKVEVPKVATQTVGGVELPVAAW |
| | | RSYLNMELTIPIFATNSDCELIVKAMQGLLKDGNPIPSAIAANSGIYEQKLI |
| | | SEEDL |
| EPN24-MCP_p6 | 12 | MHGLQDDPDLQALLKGSQLLKVKSSSWRRERFYKLQEDCKTIWQESRKVMRS |
| | | PESQLFSIEDIQEVRMGHRTEGLEKFARDIPEDRCFSIVFKDQRNTLDLIAP |
| | | SPADAQHWVQGLRKIIHHSGSMDQRQKLQSRPEPTAPPEESFRSGVETTTPP |
| | | QKQEPIDKELYPLTSLRSLFGNDPSSQASNFTQFVLVDNGGTGDVTVAPSNF |
| | | ANGVAEWISSNSRSQAYKVTCSVRQSSAQKRKYTIKVEVPKVATQTVGGVEL |
| | | PVAAWRSYLNMELTIPIFATNSDCELIVKAMQGLLKDGNPIPSAIAANSGIY |
| | | KIEELFKKHKIVAVLRANSVEEAKKKALAVFLGGVHLIEITFTVPDADTVIK |
| | | ELSFLKEMGAIIGAGTVTSVEQCRKAVESGAEFIVSPHLDEEISQFCKEKGV |
| | | FYMPGVMTPTELVKAMKLGHTILKLFPGEWGPQFVKAMKGPFPNVKFVPTG |
| | | GVNLDNVCEWFKAGVLAVGVGSALVKGTPVEVAEKAKAFVEKIRGCTEQKLI |
| | | SEEDL |
| Gag (wild-type | 13 | MGARASVLSGGELDRWEKIRLRPGGKKKYKLKHIVWASRELERFAVNPGLLE |
| HIV) | | TSEGCRQILGQLQPSLQTGSEELRSLYNTVATLYCVHQRIEIKDTKEALDKI |
| | | EEEQNKSKKKAQQAAADTGHSNQVSQNYPIVQNIQGQMVHQAISPRTLNAWV |
| | | KVVEEKAFSPEVIPMFSALSEGATPQDLNTMLNTVGGHQAAMQMLKETINEE |
| | | AAEWDRVHPVHAGPIAPGQMREPRGSDIAGTTSTLQEQIGWMTNNPPIPVGE |
| | | IYKRWIILGLNKIVRMYSPTSILDIRQGPKEPFRDYVDRFYKTLRAEQASQE |
| | | VKNWMTETLLVONANPDCKTILKALGPAATLEEMMTACQGVGGPGHKARVLA |
| | | EAMSQVTNSATIMMQRGNFRNQRKIVKCFNCGKEGHTARNCRAPRKKGCWKC |
| | | GKEGHQMKDCTERQANFLGKIWPSYKGRPGNFLQSRPEPTAPPEESFRSGVE |
| | | TTTPPQKQEPIDKELYPLTSLRSLFGNDPSSQ |
| Gag (wild-type | 14 | MGQTVTTPLSLTLGHWKDVERIAHNQSVDVKKRRWVTFCSAEWPTFNVGWPR |
| MMLV) | | DGTFNRDLITQVKIKVFSPGPHGHPDQVPYIVTWEALAFDPPPWVKPFVHPK |
| | | PPPPLPPSAPSLPLEPPRSTPPRSSLYPALTPSLGAKPKPQVLSDSGGPLID |
| | | LLTEDPPPYRDPRPPPSDRDGNGGEATPAGEAPDPSPMASRLRGRREPPVAD |
| | | STTSQAFPLRAGGNGQLQYWPFSSSDLYNWKNNNPSFSEDPGKLTALIESVL |
| | | ITHQPTWDDCQQLLGTLLTGEEKQRVLLEARKAVRGDDGRPTQLPNEVDAAF |
| | | PLERPDWDYTTQAGRNHLVHYRQLLLAGLQNAGRSPTNLAKVKGITQGPNES |
| | | PSAFLERLKEAYRRYTPYDPEDPGQETNVSMSFIWQSAPDIGRKLERLEDLK |
| | | NKTLGDLVREAEKIFNKRETPEEREERIRRETEEKEERRRTEDEQKEKERDR |
| | | RRHREMSKLLATVVSGQKQDRQGGERRRSQLDRDQCAYCKEKGHWAKDCPKK |
| | | PRGPRGPRPQTSLL |
| Gag_MHIV_MA- | 15 | MGQTVTTPLSLTLGHWKDVERIAHNQSVDVKKRRWVTFCSAEWPTFNVGWPR |
| MCP | | DGTFNRDLITQVKIKVFSPGPHGHPDQVPYIVTWEALAFDPPPWVKPFVHPK |
| | | PPPPLPPSAPSLPLEPPRSTPPRSSLYPIVQNIQGQMVHQAISPRTLNAWVK |
| | | VVEEKAFSPEVIPMFSALSEGATPQDLNTMLNTVGGHQAAMQMLKETINEEA |
| | | AEWDRVHPVHAGPIAPGQMREPRGSDIAGTTSTLQEQIGWMTNNPPIPVGEI |
| | | YKRWIILGLNKIVRMYSPTSILDIRQGPKEPFRDYVDRFYKTLRAEQASQEV |
| | | KNWMTETLLVQNANPDCKTILKALGPAATLEEMMTACQGVGGPGHKARVLAE |
| | | AMSQVTNSATIMMQRGNFRNQRKIVKCFNCGKEGHTARNCRAPRKKGCWKCG |
| | | KEGHQMKDCTERQANFLGKIWPSYKGRPGNFLQSRPEPTAPPEESFRSGVET |
| | | TTPPQKQEPIDKELYPLTSLRSLFGNDPSSQASNFTQFVLVDNGGTGDVTVA |
| | | PSNFANGVAEWISSNSRSQAYKVTCSVRQSSAQKRKYTIKVEVPKVATQTVG |
| | | GVELPVAAWRSYLNMELTIPIFATNSDCELIVKAMQGLLKDGNPIPSAIAAN |
| | | SGIY |
| Gag-MCP | 16 | MGARASVLSGGELDRWEKIRLRPGGKKKYKLKHIVWASRELERFAVNPGLLE |
| | | TSEGCRQILGQLQPSLQTGSEELRSLYNTVATLYCVHQRIEIKDTKEALDKI |
| | | EEEQNKSKKKAQQAAADTGHSNQVSQNYPIVQNIQGQMVHQAISPRTLNAWV |
| | | KVVEEKAFSPEVIPMFSALSEGATPQDLNTMLNTVGGHQAAMQMLKETINEE |
| | | AAEWDRVHPVHAGPIAPGQMREPRGSDIAGTTSTLQEQIGWMTNNPPIPVGE |
| | | IYKRWIILGLNKIVRMYSPTSILDIRQGPKEPFRDYVDRFYKTLRAEQASQE |
| | | VKNWMTETLLVQNANPDCKTILKALGPAATLEEMMTACQGVGGPGHKARVLA |
| | | EAMSQVTNSATIMMQRGNFRNQRKIVKCFNCGKEGHTARNCRAPRKKGCWKC |
| | | GKEGHQMKDCTERQANFLGKIWPSYKGRPGNFLQSRPEPTAPPEESFRSGVE |
| | | TTTPPQKQEPIDKELYPLTSLRSLFGNDPSSQASNFTQFVLVDNGGTGDVTV |
| | | APSNFANGVAEWISSNSRSQAYKVTCSVRQSSAQKRKYTIKVEVPKVATQTV |
| | | GGVELPVAAWRSYLNMELTIPIFATNSDCELIVKAMQGLLKDGNPIPSAIAA |
| | | NSGIY |
| Gag-MCPZF2 | 17 | MGARASVLSGGELDRWEKIRLRPGGKKKYKLKHIVWASRELERFAVNPGLLE |
| | | TSEGCRQILGQLQPSLQTGSEELRSLYNTVATLYCVHQRIEIKDTKEALDKI |
| | | EEEQNKSKKKAQQAAADTGHSNQVSQNYPIVQNIQGQMVHQAISPRTLNAWV |
| | | KVVEEKAFSPEVIPMFSALSEGATPQDLNTMLNTVGGHQAAMQMLKETINEE |
| | | AAEWDRVHPVHAGPIAPGQMREPRGSDIAGTTSTLQEQIGWMTNNPPIPVGE |
| | | IYKRWIILGLNKIVRMYSPTSILDIRQGPKEPFRDYVDRFYKTLRAEQASQE |
| | | VKNWMTETLLVQNANPDCKTILKALGPAATLEEMMTACQGVGGPGHKARVLA |
| | | EAMSQVTNSATIMMQRGNFRNQRKIVKCFNCGKEGHTARNCRAPRKKGCWKC |
| | | GKEGHQMKDCTEASNFTQFVLVDNGGTGDVTVAPSNFANGVAEWISSNSRSQ |
| | | AYKVTCSVRQSSAQKRKYTIKVEVPKVATQTVGGVELPVAAWRSYLNMELTI |
| | | PIFATNSDCELIVKAMQGLLKDGNPIPSAIAANSGIYROANFLGKIWPSYKG |
| | | RPGNFLQSRPEPTAPPEESFRSGVETTTPPQKQEPIDKELYPLTSLRSLFGN |
| | | DPSSQ |
| Gag-MCPΔZF2 | 18 | MGARASVLSGGELDRWEKIRLRPGGKKKYKLKHIVWASRELERFAVNPGLLE |
| | | TSEGCRQILGQLQPSLQTGSEELRSLYNTVATLYCVHQRIEIKDTKEALDKI |
| | | EEEQNKSKKKAQQAAADTGHSNQVSQNYPIVQNIQGQMVHQAISPRTLNAWV |
| | | KVVEEKAFSPEVIPMFSALSEGATPQDLNTMLNTVGGHQAAMQMLKETINEE |
| | | AAEWDRVHPVHAGPIAPGQMREPRGSDIAGTTSTLQEQIGWMTHNPPIPVGE |
| | | IYKRWIILGLNKIVRMYSPTSILDIRQGPKEPFRDYVDRFYKTLRAEQASQE |
| | | VKNWMTETLLVQNANPDCKTILKALGPGATLEEMMTACQGVGGPGHKARVLA |
| | | EAMSQVTNPATIMIQKGNFRNQRKTVKCFNCGKEGHIAKNCRAPRKKGVASN |
| | | FTQFVLVDNGGTGDVTVAPSNFANGVAEWISSNSRSQAYKVTCSVRQSSAQK |
| | | RKYTIKVEVPKVATQTVGGVELPVAAWRSYLNMELTIPIFATNSDCELIVKA |
| | | MQGLLKDGNPIPSAIAANSGIYTERQANFLGKIWPSHKGRPGNFLQSRPEPT |
| | | APPEESFRFGEETTTPSQKQEPIDKELYPLASLRSLFGSDPSSQ |
| GagZip-MCP | 19 | MGARASVLSGGELDRWEKIRLRPGGKKKYKLKHIVWASRELERFAVNPGLLE |
| | | TSEGCRQILGQLQPSLQTGSEELRSLYNTVATLYCVHQRIEIKDTKEALDKI |
| | | EEEQNKSKKKAQQAAADTGHSNQVSQNYPIVQNIQGQMVHQAISPRTLNAWV |
| | | KVVEEKAFSPEVIPMFSALSEGATPQDLNTMLNTVGGHQAAMQMLKETINEE |
| | | AAEWDRVHPVHAGPIAPGQMREPRGSDIAGTTSTLQEQIGWMTNNPPIPVGE |
| | | IYKRWIILGLNKIVRMYSPTSILDIRQGPKEPFRDYVDRFYKTLRAEQASQE |
| | | VKNWMTETLLVQNANPDCKTILKALGPAATLEEMMTACQGVGGPGHKARVLA |
| | | EAMSQVTNSATIMLQRMKQLEDKVEELLSKNYHLENEVARLKKLVGEFLGKI |
| | | WPSYKGRPGNFLQSRPEPTAPPEESFRSGVETTTPPQKQEPIDKELYPLTSL |
| | | RSLFGNDPSSQASNFTQFVLVDNGGTGDVTVAPSNFANGVAEWISSNSRSQA |
| | | YKVTCSVRQSSAQKRKYTIKVEVPKVATQTVGGVELPVAAWRSYLNMELTIP |
| | | IFATNSDCELIVKAMQGLLKDGNPIPSAIAANSGIY |
| GagZip-MCP- | 20 | MGARASVLSGGELDRWEKIRLRPGGKKKYKLKHIVWASRELERFAVNPGLLE |
| dpol | | TSEGCRQILGQLQPSLQTGSEELRSLYNTVATLYCVHQRIEIKDTKEALDKI |
| | | EEEQNKSKKKAQQAAADTGHSNQVSQNYPIVQNIQGQMVHQAISPRTLNAWV |
| | | KVVEEKAFSPEVIPMFSALSEGATPQDLNTMLNTVGGHQAAMQMLKETINEE |
| | | AAEWDRVHPVHAGPIAPGQMREPRGSDIAGTTSTLQEQIGWMTNNPPIPVGE |
| | | IYKRWIILGLNKIVRMYSPTSILDIRQGPKEPFRDYVDRFYKTLRAEQASQE |
| | | VKNWMTETLLVQNANPDCKTILKALGPAATLEEMMTACQGVGGPGHKARVLA |
| | | EAMSQVTNSATIMMQRGNFRNQRKIVKCFNCGKEGHTARNCRAPRKKGCWKC |
| | | GKEGHQMKDCTERQANFLGKIWPSYKGRPGNFLQSRPEPTAPPEESFRSGVE |
| | | TTTPPQKQEPIDKELYPLTSLRSLFGNDPSSQASNFTQFVLVDNGGTGDVTV |
| | | APSNFANGVAEWISSNSRSQAYKVTCSVRQSSAQKRKYTIKVEVPKVATQTV |
| | | GGVELPVAAWRSYLNMELTIPIFATNSDCELIVKAMQGLLKDGNPIPSAIAA |
| | | NSGIY |
| GagZip-MCPZip | 21 | MGARASVLSGGELDRWEKIRLRPGGKKKYKLKHIVWASRELERFAVNPGLLE |
| | | TSEGCRQILGQLQPSLQTGSEELRSLYNTVATLYCVHQRIEIKDTKEALDKI |
| | | EEEQNKSKKKAQQAAADTGHSNQVSQNYPIVQNIQGQMVHQAISPRTLNAWV |
| | | KVVEEKAFSPEVIPMFSALSEGATPQDLNTMLNTVGGHQAAMQMLKETINEE |
| | | AAEWDRVHPVHAGPIAPGQMREPRGSDIAGTTSTLQEQIGWMTNNPPIPVGE |
| | | IYKRWIILGLNKIVRMYSPTSILDIRQGPKEPFRDYVDRFYKTLRAEQASQE |
| | | VKNWMTETLLVQNANPDCKTILKALGPAATLEEMMTACQGVGGPGHKARVLA |
| | | EAMSQVTNSATIMLQRMKQLEDKVEELLSKNYHLENEVARLKKLVGEASNFT |
| | | QFVLVDNGGTGDVTVAPSNFANGVAEWISSNSRSQAYKVTCSVRQSSAQKRK |
| | | YTIKVEVPKVATQTVGGVELPVAAWRSYLNMELTIPIFATNSDCELIVKAMQ |
| | | GLLKDGNPIPSAIAANSGIYFLGKIWPSYKGRPGNFLQSRPEPTAPPEESFR |
| | | SGVETTTPPQKQEPIDKELYPLTSLRSLFGNDPSSQ |
| GagZip-Δp1p6- | 22 | MGARASVLSGGELDRWEKIRLRPGGKKKYKLKHIVWASRELERFAVNPGLLE |
| MCP | | TSEGCRQILGQLQPSLQTGSEELRSLYNTVATLYCVHQRIEIKDTKEALDKI |
| | | EEEQNKSKKKAQQAAADTGHSNQVSQNYPIVQNIQGQMVHQAISPRTLNAWV |
| | | KVVEEKAFSPEVIPMFSALSEGATPQDLNTMLNTVGGHQAAMQMLKETINEE |
| | | AAEWDRVHPVHAGPIAPGQMREPRGSDIAGTTSTLQEQIGWMTNNPPIPVGE |
| | | IYKRWIILGLNKIVRMYSPTSILDIRQGPKEPFRDYVDRFYKTLRAEQASQE |
| | | VKNWMTETLLVQNANPDCKTILKALGPAATLEEMMTACQGVGGPGHKARVLA |
| | | EAMSQVTNSATIMLQRMKQLEDKVEELLSKNYHLENEVARLKKLVGEASNFT |
| | | QFVLVDNGGTGDVTVAPSNFANGVAEWISSNSRSQAYKVTCSVRQSSAQKRK |
| | | YTIKVEVPKVATQTVGGVELPVAAWRSYLNMELTIPIFATNSDCELIVKAMQ |
| | | GLLKDGNPIPSAIAANSGIY |
| MiniGagZip- | 23 | MGARASVSPTSILDIRQGPKEPFRDYVDRFYKTLRAEQASQEVKNWMTETLL |
| MCP | | VQNANPDCKTILKALGPAATLEEMMTACQGVGGPGHKARVLAEAMSQVTNSA |
| | | TIMLQRMKQLEDKVEELLSKNYHLENEVARLKKLVGELQSRPEPTAPPEESF |
| | | RSGVETTTPPQKQEPIDKELYPLTSLRSLFGNDPSSQASNFTQFVLVDNGGT |
| | | GDVTVAPSNFANGVAEWISSNSRSQAYKVTCSVRQSSAQKRKYTIKVEVPKV |
| | | ATQTVGGVELPVAAWRSYLNMELTIPIFATNSDCELIVKAMQGLLKDGNPIP |
| | | SAIAANSGIY |
| MiniGagZip- | 24 | MGARASVSPTSILDIRQGPKEPFRDYVDRFYKTLRAEQASQEVKNWMTETLL |
| MCPZip | | VQNANPDCKTILKALGPAATLEEMMTACQGVGGPGHKARVLAEAMSQVTNSA |
| | | TIMLQRMKQLEDKVEELLSKNYHLENEVARLKKLVGEASNFTQFVLVDNGGT |
| | | GDVTVAPSNFANGVAEWISSNSRSQAYKVTCSVRQSSAQKRKYTIKVEVPKV |
| | | ATQTVGGVELPVAAWRSYLNMELTIPIFATNSDCELIVKAMQGLLKDGNPIP |
| | | SAIAANSGIY |

TABLE 2
RNA EXPORTERS AND EXPORTED RNA REPORTERS

| Name | Component | Description |
|---|---|---|
| Gag | Exporter | Gag protein from HIV (Gag) |
| Gag-MCP | Exporter | Fusion of Gag and MS2 coat protein (MCP) with MCP |
| | | located after C-terminus of Gag |
| Gag-MCP_ZF2 | Exporter | Fusion of Gag and MCP with MCP located after second |
| | | zinc finger domain of Gag (ZF2) |
| Gag_dZF2-MCP | Exporter | Fusion of Gag and MCP with ZF2 deleted from Gag and |
| | | replaced by MCP |
| GagZip-MCP | Exporter | Fusion of Gag and MCP with nucleocapsid domain |
| | | deleted from Gag and replaced by leucine zipper from |
| | | GCN4 (GagZip), and MCP located after C-terminus of |
| | | Gag |
| GagZip-MCP_Zip | Exporter | Fusion of Gag and MCP with nucleocapsid domain |
| | | deleted from Gag and replaced by leucine zipper from |
| | | GCN4, and MCP located after leucine zipper |
| GagZip-dp1dp6-MCP | Exporter | Fusion of Gag and MCP with p1p6 domain deleted from |
| | | Gag, nucleocapsid domain deleted from Gag and replaced |
| | | by leucine zipper from GCN4, and MCP located after |
| | | leucine zipper |
| GagZip-MCP-dpol | Exporter | Fusion of Gag and MCP with nucleocapsid domain |
| | | deleted from Gag and replaced by leucine zipper from |
| | | GCN4 (GagZip), and MCP located after C-terminus of |
| | | Gag; coding sequence of Gag recoded to remove slippery |
| | | frameshift sequence at natural junction with Pol |
| MiniGagZip-MCP | Exporter | Fusion of Gag and MCP with portions of matrix, capsid, |
| | | and p1 domains deleted; nucleocapsid domain deleted and |
| | | replaced by leucine zipper from GCN4; and MCP located |
| | | after C terminus of Gag |
| MiniGagZip- | Exporter | Fusion of Gag and MCP with portions of matrix, capsid, |
| MCP_Zip | | and p1 domains deleted; nucleocapsid domain deleted and |
| | | replaced by leucine zipper from GCN4; and MCP located |
| | | after leucine zipper |
| mCherry | Reporter | mRNA encoding mCherry fluorescent protein |
| mCherry-Psi | Reporter | mRNA encoding mCherry fluorescent protein with Psi |
| | | packaging signal sequence from HIV in 3′ untranslated |
| | | region (UTR) |
| mCherry-MS2x2 | Reporter | mRNA encoding mCherry fluorescent protein with two |
| | | tandem repeats of MS2 stem-loop aptamer in 3′ UTR |
| mCherry-MS2x4 | Reporter | mRNA encoding mCherry fluorescent protein with four |
| | | tandem repeats of MS2 stem-loop aptamer in 3′ UTR |
| mCherry-MS2x6 | Reporter | mRNA encoding mCherry fluorescent protein with six |
| | | tandem repeats of MS2 stem-loop aptamer in 3′ UTR |
| mCherry-MS2x8 | Reporter | mRNA encoding mCherry fluorescent protein with eight |
| | | tandem repeats of MS2 stem-loop aptamer in 3′ UTR |
| mCherry-MS2x12 | Reporter | mRNA encoding mCherry fluorescent protein with twelve |
| | | tandem repeats of MS2 stem-loop aptamer in 3′ UTR |

The reporter cells can comprise cells situated in an organ and/or tissue, e.g., an organ and/or tissue or a subject (e.g., different organs and/or tissues of a subject). The population of reporter cells can comprise a plurality of reporter cells that differ with respect to cell type and/or cell state. The one or more reporter RNA molecule(s) can comprise a plurality of reporter RNA molecules. Each of the reporter RNA molecules can comprise a unique reporter barcode indicating a unique cell type and/or a unique cell state of the reporter cell from which it is derived. The plurality of reporter RNA molecules can comprise at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, or a number or a range between any two of these values, different reporter RNA molecules each comprising a unique reporter barcode. The presence and/or amount of an exported reporter RNA molecule comprising a unique reporter barcode can be correlated with the presence and/or amount of the unique cell type and/or a unique cell state in said reporter cell. The degree of expression and/or degradation of the plurality of reporter RNA molecules can be associated with the presence and/or amount of the unique cell type and/or a unique cell state. One or more second promoter(s) can be operably linked to each of the one or more second polynucleotide(s) each encoding one or more reporter RNA molecule(s). A second promoter can be capable of inducing transcription of a second polynucleotide to generate a reporter RNA molecule comprising a unique reporter barcode depending on the presence and/or amount of a unique cell type and/or a unique cell state associated with said unique reporter barcode. Second promoter(s) can be transcription factor-dependent, signal-responsive, metabolic, and/or circadian promoters. The one or more second polynucleotide(s) can comprise one or more silencer effector binding sequences. The silencer effector can comprise a microRNA (miRNA), a precursor microRNA (pre-miRNA), a small interfering RNA (siRNA), a short-hairpin RNA (shRNA), precursors thereof, derivatives thereof, or a combination thereof. Said silencer effector can be capable of binding the one or more silencer effector binding sequences, thereby reducing the stability of reporter RNA molecule(s). In some embodiments, the expression and/or activity of the silencer effector configured to be responsive to changes in cell state and/or cell type. The reporter cell can comprise a circuit configured to regulate the expression and/or stability of the plurality of reporter RNA molecules in response to the cell type and/or cell state of the reporter cell, optionally said circuit comprises one or more components encoded by one or more fifth polynucleotide(s).

Synthetic biology allows for rational design of circuits that confer new functions in living cells. Many natural cellular functions are implemented by protein-level circuits, in which proteins specifically modify each other's activity, localization, or stability. Synthetic protein circuits have been described in, Gao, Xiaojing J., et al. “Programmable protein circuits in living cells.” Science 361.6408 (2018): 1252-1258; and WO2019/147478; the content of each of these, including any supporting or supplemental information or material, is incorporated herein by reference in its entirety. In some embodiments, synthetic protein circuits respond to inputs only above or below a certain tunable threshold concentration, such as those provided in US2020/0277333, the content of which is incorporated herein by reference in its entirety. In some embodiments, synthetic protein circuits comprise one or more synthetic protein circuit design components and/or concepts of US2020/0071362, the content of which is incorporated herein by reference in its entirety. In some embodiments, synthetic protein circuits comprise rationally designed circuits, including miRNA-level and/or protein-level incoherent feed-forward loop circuits, that maintain the expression of a payload at an efficacious level, such as those provided in US2021/0171582, the content of which is incorporated herein by reference in its entirety. The compositions, methods, systems and kits provided herein can be employed in concert with those described in International Patent Application No. PCT/US2021/048100, entitled “Synthetic Mammalian Signaling Circuits For Robust Cell Population Control” filed on Aug. 27, 2021, the content of which is incorporated herein by reference in its entirety. Said reference discloses circuits, compositions, nucleic acids, populations, systems, and methods enabling cells to sense, control, and/or respond to their own population size and can be employed with the circuits provided herein. In some embodiments, an orthogonal communication channel allows specific communication between engineered cells. Also described therein, in some embodiments, is an evolutionarily robust ‘paradoxical’ regulatory circuit architecture in which orthogonal signals both stimulate and inhibit net cell growth at different signal concentrations. In some embodiments, engineered cells autonomously reach designed densities and/or activate therapeutic or safety programs at specific density thresholds. The systems, methods, compositions, and kits provided herein can, in some embodiments, be employed in concert with the systems, methods, compositions, and kits described in PCT Patent Application Publication No. WO2022/125590, entitled, “A synthetic circuit for cellular multistability,” the content of which is incorporated herein by reference in its entirety. The systems, methods, compositions, and kits provided herein can, in some embodiments, be employed in concert with the systems, methods, compositions, and kits described in U.S. Patent Application No. 2018/0142307 and 2020/0172968, the contents of which are incorporated herein by reference in their entirety.

A unique cell type and/or a unique cell state can comprise a unique gene expression pattern, e.g. the unique cell type and/or unique cell state can comprise a unique anatomic location. The unique cell type and/or the unique cell state can comprise anatomically locally unique gene expression. A unique cell type and/or a unique cell state can be caused by hereditable, environmental, and/or idiopathic factors. In some embodiments, the unique cell type and/or the cell in the unique cell state (i) causes and/or aggravates a disease or disorder and/or (ii) is associated with the pathology of a disease or disorder. The unique cell state can comprise a senescent cell state induced by a tumor microenvironment. The senescent cell state induced by a tumor microenvironment can comprise expression of CD57, KRLG1, TIGIT, or any combination thereof. The unique cell state and/or unique cell type can be characterized by aberrant signaling of one or more signal transducer(s). In some embodiments, the unique cell state comprises: a physiological state (e.g., a cell cycle state, a differentiation state, a development state a metabolic state, or a combination thereof); and/or a pathological state (e.g., a disease state, a human disease state, a diabetic state, an immune disorder state, a neurodegenerative disorder state, an oncogenic state, or a combination thereof).

The unique cell state and/or unique cell type can be characterized by one or more of cell proliferation, stress pathways, oxidative stress, stress kinase activation, DNA damage, lipid metabolism, carbohydrate regulation, metabolic activation including Phase I and Phase II reactions, Cytochrome P-450 induction or inhibition, ammonia detoxification, mitochondrial function, peroxisome proliferation, organelle function, cell cycle state, morphology, apoptosis, DNA damage, metabolism, signal transduction, cell differentiation, cell-cell interaction and cell to non-cellular compartment.

The unique cell state and/or unique cell type can be characterized by one or more of acute phase stress, cell adhesion, AH-response, anti-apoptosis and apoptosis, antimetabolism, anti-proliferation, arachidonic acid release, ATP depletion, cell cycle disruption, cell matrix disruption, cell migration, cell proliferation, cell regeneration, cell-cell communication, cholestasis, differentiation, DNA damage, DNA replication, early response genes, endoplasmic reticulum stress, estogenicity, fatty liver, fibrosis, general cell stress, glucose deprivation, growth arrest, heat shock, hepatotoxicity, hypercholesterolemia, hypoxia, immunotox, inflammation, invasion, ion transport, liver regeneration, cell migration, mitochondrial function, mitogenesis, multidrug resistance, nephrotoxicity, oxidative stress, peroxisome damage, recombination, ribotoxic stress, sclerosis, steatosis, teratogenesis, transformation, disrupted translation, transport, and tumor suppression.

The unique cell state and/or unique cell type can be characterized by one or more of nutrient deprivation, hypoxia, oxidative stress, hyperproliferative signals, oncogenic stress, DNA damage, ribonucleotide depletion, replicative stress, and telomere attrition, promotion of cell cycle arrest, promotion of DNA-repair, promotion of apoptosis, promotion of genomic stability, promotion of senescence, and promotion of autophagy, regulation of cell metabolic reprogramming, regulation of tumor microenvironment signaling, inhibition of cell stemness, survival, and invasion.

In some embodiments, the cell type is: an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof. The stem cell can comprise an embryonic stem cell, an induced pluripotent stem cell (iPSC), a hematopoietic stem/progenitor cell (HSPC), or any combination thereof.

The LNs contacted with RNase can be capable of protecting reporter RNA molecule(s) comprised therein from RNase-mediated degradation (e.g., in the absence of detergent). The average diameter of the LNs of the population of LNs can be about 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, 205 nm, 210 nm, 215 nm, 220 nm, 225 nm, 230 nm, 235 nm, 240 nm, 245 nm, 250 nm, 300 nm, 400 nm, or 500 nm. The average can be the mean (e.g., arithmetic mean, geometric mean, and/or harmonic mean), median or mode. In some embodiments, the LNs have a minimum diameter and/or a maximum diameter of about 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, 205 nm, 210 nm, 215 nm, 220 nm, 225 nm, 230 nm, 235 nm, 240 nm, 245 nm, 250 nm, 300 nm, 400 nm, or 500 nm. The diameter can be hydrodynamic diameter, e.g., as measured by dynamic light scattering (DLS).

In some embodiments, the population of LNs differ from each other with respect the RNA contents. The LNs can comprise a lipid bilayer (e.g., a lipid bilayer derived from the reporter cell from which the LN was secreted). The reporter cells can be situated in a tissue. The methods provided herein can comprise administering nucleic acid composition(s) to a subject (e.g., a mammal) to generate reporter cells in vivo. In some embodiments, reporter RNA molecule(s) encode a dosage indicator protein. The dosage indicator protein can be detectable. The dosage indicator protein can comprise green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCherry, mruby3, rsCherry, rsCherryRev, derivatives thereof, or any combination thereof. The LNs can comprise exported endogenous RNA molecule(s). The endogenous RNA molecule(s) can comprise one or more of messenger RNAs (mRNAs), microRNAs, small interfering RNAs (siRNAs), and RNA degradation products. In some embodiments, the endogenous RNA molecule(s) are not mitochondrial RNA molecules. The exported endogenous RNA molecule(s) can comprise an unbiased sample of the non-mitochondrial transcriptome of the reporter cell. The packing signal(s) can comprise an array of at least about 2, 3, 4, 5, 6, 7, 8, 9, 10, 11, 12, 13, or 14, tandem repeats of an aptamer (e.g., a MS2 stem-loop aptamer). In some embodiments, the relative abundance of endogenous RNA molecules to reporter RNA molecule(s) in secreted LNs can be configured by varying the number of tandem repeats.

One or more of the first polynucleotide(s), the second polynucleotide(s), the third polynucleotide(s), the fourth polynucleotide(s), and the fifth polynucleotide(s), can be operably connected to a promoter selected from the group comprising: a minimal promoter, optionally TATA, miniCMV, and/or miniPromo; a ubiquitous promoter; a tissue-specific promoter and/or a lineage-specific promoter; and/or a ubiquitous promoter, optionally a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CASI promoter, a CBH promoter, or any combination thereof.

The nucleic acid composition can be complexed or associated with one or more lipids or lipid-based carriers, thereby forming liposomes, lipid nanoparticles (LNPs), lipoplexes, and/or nanoliposomes, optionally encapsulating the nucleic acid composition. In some embodiments, the nucleic acid composition is, comprises, or further comprises, one or more vectors. At least one of the one or more vectors can be a viral vector, a plasmid, a transposable element, a naked DNA vector, a lipid nanoparticle (LNP), or any combination thereof. The viral vector can be an AAV vector, a lentivirus vector, a retrovirus vector, an adenovirus vector, a herpesvirus vector, a herpes simplex virus vector, a cytomegalovirus vector, a vaccinia virus vector, a MVA vector, a baculovirus vector, a vesicular stomatitis virus vector, a human papillomavirus vector, an avipox virus vector, a Sindbis virus vector, a VEE vector, a Measles virus vector, an influenza virus vector, a hepatitis B virus vector, an integration-deficient lentivirus (IDLV) vector, or any combination thereof. The transposable element can be piggybac transposon or sleeping beauty transposon. The polynucleotide(s) encoding the RNA exporter protein, the reporter RNA molecule(s), and/or the packing RNA molecule(s) can be comprised in the one or more vectors. The polynucleotide(s) encoding the RNA exporter protein, the reporter RNA molecule(s), and/or the packing RNA molecule(s) can be comprised in the same vector and/or different vectors. The polynucleotide(s) encoding the RNA exporter protein, the reporter RNA molecule(s), and/or the packing RNA molecule(s) can be situated on the same nucleic acid and/or different nucleic acids. The nucleic acid compositions provided herein can include libraries of vectors comprising cell barcode(s) that are the same or different.

Vectors provided herein include integrating vectors and non-integrating vectors. Integrating vectors have their delivered RNA/DNA permanently incorporated into the host cell chromosomes. Non-integrating vectors remain episomal which means the nucleic acid contained therein is never integrated into the host cell chromosomes. Examples of integrating vectors include retroviral vectors, lentiviral vectors, hybrid adenoviral vectors, and herpes simplex viral vector. One example of a non-integrative vector is a non-integrative viral vector. Non-integrative viral vectors eliminate the risks posed by integrative retroviruses, as they do not incorporate their genome into the host DNA. One example is the Epstein Barr oriP/Nuclear Antigen-1 (“EBNA1”) vector, which is capable of limited self-replication and known to function in mammalian cells. As containing two elements from Epstein-Barr virus, oriP and EBNA1, binding of the EBNA1 protein to the virus replicon region oriP maintains a relatively long-term episomal presence of plasmids in mammalian cells. This particular feature of the oriP/EBNA1 vector makes it ideal for generation of integration-free iPSCs. Another non-integrative viral vector is adenoviral vector and the adeno-associated viral (AAV) vector. Other non-integrative viral vectors contemplated herein are single-strand negative-sense RNA viral vectors, such Sendai viral vector and rabies viral vector. Another example of a non-integrative vector is a minicircle vector. Minicircle vectors are circularized vectors in which the plasmid backbone has been released leaving only the eukaryotic promoter and cDNA(s) that are to be expressed. As used herein, the term “viral vector” refers to a nucleic acid vector construct that includes at least one element of viral origin and has the capacity to be packaged into a viral vector particle. The viral vector can contain a nucleic acid encoding a polypeptide as described herein in place of nonessential viral genes. The vector and/or particle may be utilized for the purpose of transferring nucleic acids into cells either in vitro or in vivo. Numerous forms of viral vectors are known in the art.

Disclosed herein include systems for export of reporter RNA molecules. In some embodiments, the kit comprises: an RNA exporter protein provided herein; and one or more reporter RNA molecule(s) disclosed herein. Disclosed herein include systems for non-destructive live continuous cell measurement of cell state and/or cell type. In some embodiments, the kit comprises: an RNA exporter protein provided herein; and one or more reporter RNA molecule(s) disclosed herein. The system can comprise: one or more packing RNA molecule(s) disclosed herein.

Disclosed herein include populations of lipid-enveloped nanoparticles (LNs). In some embodiments, the population of LNs comprise: an RNA exporter protein provided herein; and one or more reporter RNA molecule(s) disclosed herein. The population of LNs can comprise: one or more packing RNA molecule(s) disclosed herein. The population of LNs can be derived from expression of a nucleic acid composition disclosed herein.

Disclosed herein include methods for determining the cell type and/or cell state of one or more reporter cells. In some embodiments, the method comprises: providing a population of reporter cells provided herein; isolating a plurality of exported reporter RNA molecule(s) at one or more time points; and obtaining sequence information of the plurality of exported reporter RNA molecule(s), or products thereof, to determine the cell type and/or cell state of the reporter cell(s).

The method can comprise: isolating a plurality of exported packing RNA molecule(s) at one or more time points; and obtaining sequence information of the plurality of exported packing RNA molecule(s), or products thereof, to determine the cell type and/or cell state of the reporter cell(s).

The providing step can comprise incubation of the reporter cell(s). In some embodiments, one or more reporter cell(s) divide and/or die during said incubation. The providing step can comprise introducing into the reporter cell(s) nucleic acid composition(s) comprising the one or more first polynucleotide(s), the one or more second polynucleotide(s), the one or more third polynucleotide(s), the one or more fourth polynucleotide(s), and/or the one or more fifth polynucleotide(s). In some embodiments, the one or more first polynucleotide(s), the one or more second polynucleotide(s), the one or more third polynucleotide(s), the one or more fourth polynucleotide(s), and/or the one or more fifth polynucleotide(s) become integrated in the genome of the reporter cell(s) after the introducing step. In some embodiments, providing the reporter cells can comprise transducing reporter cells with a library of vectors encoding one or more cell barcode(s) at a multiplicity of infection (MOI) configured to increase the likelihood that each cell is transduced with only a single vector, e.g., an MOI between about 0.3 and 0.75. In some embodiments, each of the library members of the library of vectors encodes cell barcode(s) of a different sequence.

The method can comprise exposing the reporter cell(s) to one or more agents before, during, and/or after the one or more time points. In some embodiments, the one or more agents comprise: (i) one or more of a chemical agent, a pharmaceutical, small molecule, a biologic, a CRISPR single-guide RNA (sgRNA), a small interfering RNA (siRNA), CRISPR RNA (crRNA), a small hairpin RNA (shRNA), a microRNA (miRNA), a piwi-interacting RNA (piRNA), an antisense oligonucleotide, a peptide or peptidomimetic inhibitor, an aptamer, an antibody, an intrabody, or any combination thereof; (ii) an expression vector, wherein the expression vector encodes one or more of the following: an mRNA, an antisense nucleic acid molecule, a RNAi molecule, a shRNA, a mature miRNA, a pre-miRNA, a pri-miRNA, an anti-miRNA, a ribozyme, any combination thereof; (iii) an infectious agent, an anti-infectious agent, or a mixture thereof; (iv) a cytotoxic agent (e.g., a chemotherapeutic agent, a biologic agent, a toxin, a radioactive isotope, or any combination thereof); (v) one or more of an epigenetic modifying agent, epigenetic enzyme, a bicyclic peptide, a transcription factor, a DNA or protein modification enzyme, a DNA-intercalating agent, an efflux pump inhibitor, a nuclear receptor activator or inhibitor, a proteasome inhibitor, a competitive inhibitor for an enzyme, a protein synthesis inhibitor, a nuclease, a protein fragment or domain, a tag or marker, an antigen, an antibody or antibody fragment, a ligand or a receptor, a synthetic or analog peptide from a naturally-bioactive peptide, an anti-microbial peptide, a pore-forming peptide, a targeting or cytotoxic peptide, a degradation or self-destruction peptide, a CRISPR component system or component thereof, DNA, RNA, artificial nucleic acids, a nanoparticle, an oligonucleotide aptamer, a peptide aptamer, or any combination thereof; and/or (vi) at least one effector activity selected from the group consisting of: modulating a biological activity, binding a regulatory protein, modulating enzymatic activity, modulating substrate binding, modulating receptor activation, modulating protein stability/degradation, modulating transcript stability/degradation, or any combination thereof.

The isolating step can comprise isolating LNs and/or extracting RNA to generate a plurality of RNA molecules. In some embodiments, the isolating step comprises: separating cells from the extracellular environment to generate a supernatant; clarifying the supernatant; and extracting RNA from the supernatant to generate a plurality of RNA molecules. In some embodiments, clarifying the supernatant comprises: centrifuging the supernatant (e.g., centrifugation at 3000 g for 5 minutes); and/or filtering the supernatant through a filter (e.g., a 0.45 um filter).

The method can comprise: contacting a first strand primer with the plurality of RNA molecules, optionally the first strand primer is a target-specific primer, further optionally the target-specific primer comprises oligo(dT) and/or a first universal sequence. The method can comprise: conducting a first strand synthesis reaction using a reverse transcriptase to generate a plurality of first strand synthesis products. Obtaining sequence information can comprise obtaining sequence information of the plurality of first strand synthesis products, or products thereof. The method can comprise: amplifying the plurality of first strand synthesis products using a first amplification primer and a second amplification primer, thereby generating a plurality of amplicons. Obtaining sequence information can comprise obtaining sequence information of the plurality of amplicons, or products thereof. Amplifying the plurality of first strand synthesis products can comprise adding sequences of binding sites of sequencing primers and/or sequencing adaptors, complementary sequences thereof, and/or portions thereof, to the plurality of first strand synthesis products. The sequencing adaptors can comprise a P5 sequence, a P7 sequence, complementary sequences thereof, and/or portions thereof. The sequencing primers can comprise a Read 1 sequencing primer, a Read 2 sequencing primer, complementary sequences thereof, and/or portions thereof. The first amplification primer and the second amplification primer can be target-specific primers. The first amplification primer can be capable of hybridizing to the first universal sequence, or a complement thereof, and the second amplification primer can be a target-specific primer. The target-specific primers can be configured to amplify the reporter barcode(s) and/or cell barcode(s).

The method can comprise: adding spike-in RNA molecules of known quantity to the supernatant, extracted RNA, or isolated LNs; and obtaining sequence information of the spike-in RNA molecules, or products thereof, to determine relative abundance of exported reporter RNA molecule(s) and/or endogenous RNA molecule(s) across samples of reporter cells. In some embodiments, the spike-in RNA molecules are not homologous to genomic sequences of the reporter cell(s). Spike-in RNA molecules can be homologous to genomic sequences of a species, optionally the species is a non-mammalian species. The non-mammalian species can be a phage species, optionally said phage species is T7 phage, a PhiX phage, or any combination thereof.

In some embodiments, obtaining sequence information comprises: obtaining sequencing data comprising a plurality of sequencing reads of the plurality of exported reporter RNA molecule(s), the plurality of exported package RNA molecule(s), the plurality of first strand synthesis products, the plurality of amplicons, and/or products thereof, wherein each of the plurality of sequencing reads comprise at least one cell barcode sequence, and a reporter barcode sequence.

The method can comprise: for each unique lineage barcode sequence, which indicates a single reporter cell of the population of reporter cells: detecting the presence and/or amount of each reporter barcode of the plurality of reporter barcodes, thereby generating a profile of each reporter cell, wherein the profile comprises collective cell type(s) and cell state(s) of said reporter cell. In some embodiments, the method further comprises performing phylogenetic reconstruction to determine lineage dynamics.

The method can comprise: for each unique subpopulation barcode sequence, which indicates a single subpopulation of reporter cells of the population of reporter cells: detecting the presence and/or amount of each reporter barcode of the plurality of reporter barcodes, thereby generating a profile of each subpopulation, wherein the profile comprises collective cell type(s) and cell state(s) of said subpopulation.

The method can comprise: for each unique clone barcode sequence, which indicates a clonal population of the population of reporter cells: detecting the presence and/or amount of each reporter barcode of the plurality of reporter barcodes, thereby generating a profile of each clonal population, wherein the profile comprises collective cell type(s) and cell state(s) of said clonal population.

The one or more time points can comprise a plurality of time points. The method can comprise longitudinally monitoring single-cell state dynamics, subpopulation dynamics, clonal population dynamics, and/or lineage dynamics, optionally one or more lineages do not exist at the last time point. The method can comprise further identifying genetic or molecular perturbations which affect single-cell state dynamics, subpopulation dynamics, clonal population dynamics, and/or lineage dynamics. The genetic or molecular perturbations can be associated with a disease or disorder, or treatment thereof.

In some embodiments, the LNs comprise exported endogenous RNA molecule(s) and can further comprise reporter RNA molecule(s) and/or packing RNA molecule(s) comprising cell barcode(s). In some embodiments, the method comprises physically linking said cell barcode(s) to said endogenous RNA molecule(s), e.g., via ligation, polymerization, primer extension, or physical co-compartmentalization.

The method can comprise: obtaining sequence information of the endogenous RNA molecule(s), or product thereof. In some embodiments, obtaining sequence information comprises: immobilizing individual LNs of the population of LNs on a surface; and performing fluorescence in situ hybridization or in situ sequencing.

In some embodiments, obtaining sequence information of the endogenous RNA molecule(s), or products thereof, comprises: obtaining sequencing data comprising a plurality of sequencing reads of the endogenous RNA molecule(s), or products thereof, wherein each of the plurality of sequencing reads comprise at least one cell barcode sequence, and a sequence of at least a portion of an endogenous RNA molecule.

The method can comprise: for each unique lineage barcode sequence, which indicates a single reporter cell of the population of reporter cells: detecting the presence and/or amount of endogenous RNA molecules, thereby generating a transcriptomic profile of each reporter cell.

The method can comprise: for each unique subpopulation barcode sequence, which indicates a single subpopulation of reporter cells of the population of reporter cells: detecting the presence and/or amount of endogenous RNA molecules, thereby generating a transcriptomic profile of each subpopulation.

The method can comprise: for each unique clone barcode sequence, which indicates a clonal population of the population of reporter cells: detecting the presence and/or amount of endogenous RNA molecules, thereby generating a transcriptomic profile of each clonal population.

The one or more time points can comprise a plurality of time points, and the method can comprise longitudinally monitoring single-cell transcriptome dynamics.

Disclosed herein include methods for generating an enriched population of lipid-enveloped nanoparticles (LNs). In some embodiments, the method comprises: providing a population of reporter cells provided herein, wherein the reporter cells secrete LNs comprising an affinity tag; and enriching the LNs using the affinity tag, optionally via a column, bead, and/or continuous flow. The LNs can comprise an affinity tag and the method can comprise enriching the LNs using the affinity tag, optionally via a column, bead, and/or continuous flow. In some embodiments, the affinity tag is present on the surface of the LNs, the affinity tag is fused to the RNA exporter protein or the affinity tag is separate from the RNA exporter protein, and/or the affinity tag is selected from the group comprising biotin, azido group, acetylene group, HIS-tag, Calmodulin-tag, CBP, CYD, Strep II, FLAG-tag, HA-tag, Myc-tag, S-tag, SBP-tag, Softag-1, Softag-3, V5-tag, Xpress-tag, Isopeptag, SpyTag, B, HPC peptide tags, GST, MBP, biotin carboxyl carrier protein, glutathione-S-transferase-tag, green fluorescent protein-tag, maltose binding protein-tag, Nus-tag, Strep-tag, and thioredoxin-tag.

### EXAMPLES

Some aspects of the embodiments discussed above are disclosed in further detail in the following examples, which are not in any way intended to limit the scope of the present disclosure.

### Example 1

### Exported RNA Reporters for Live-Cell Measurement

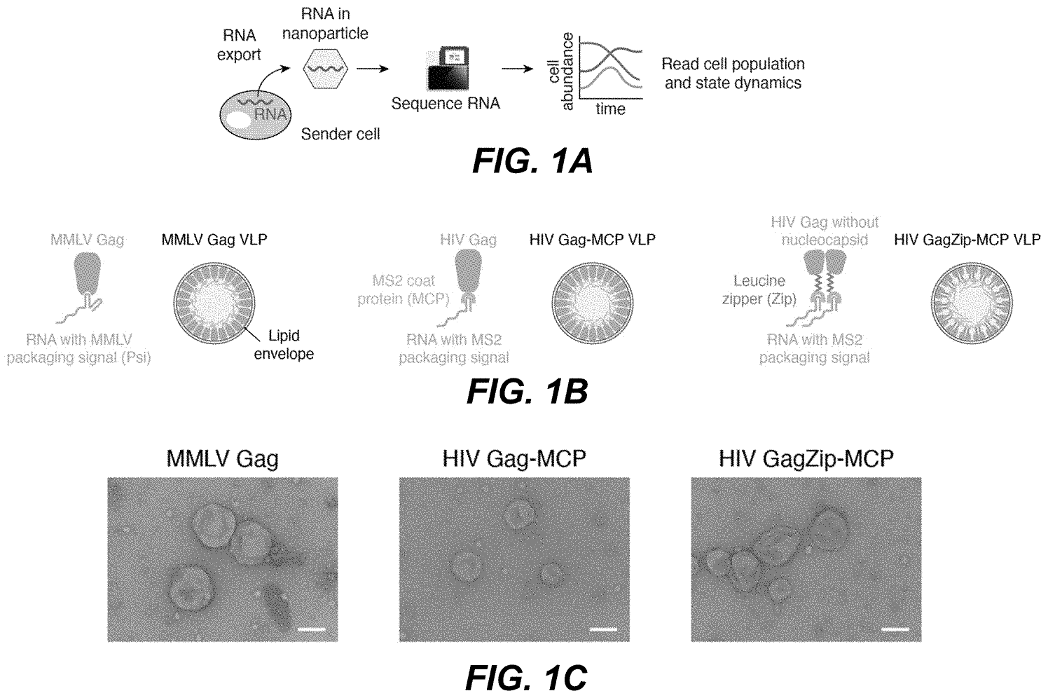

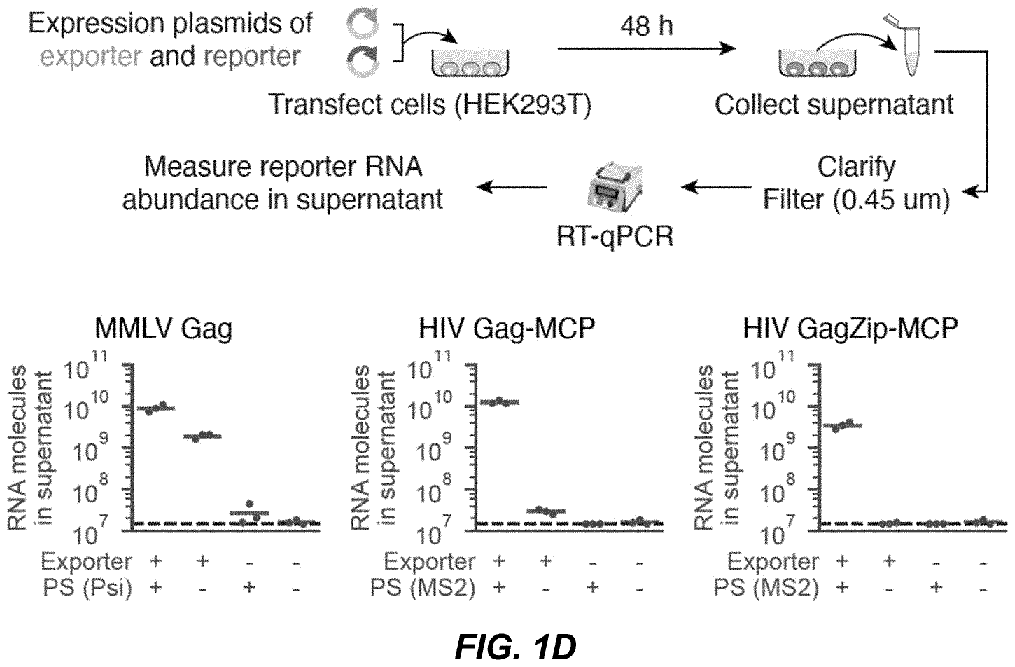

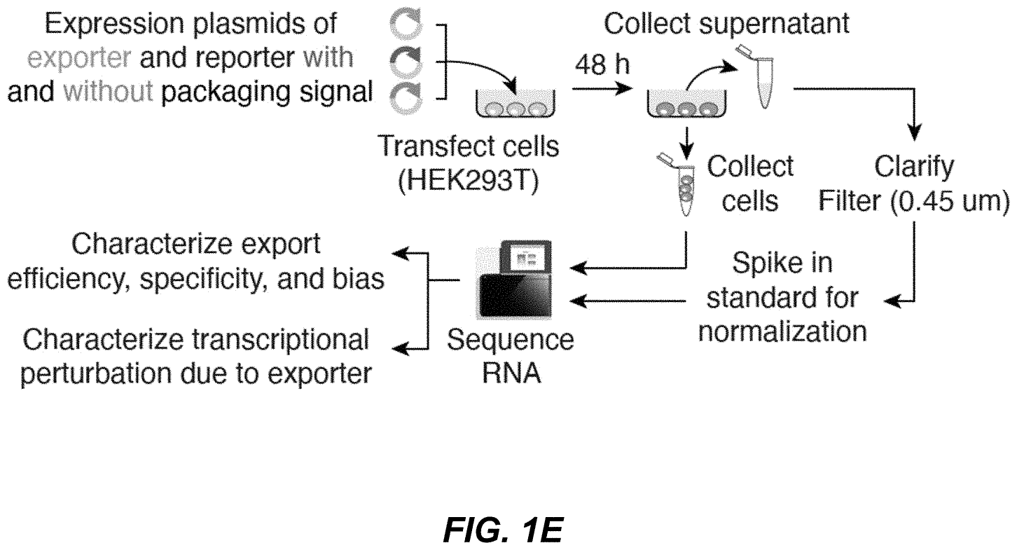

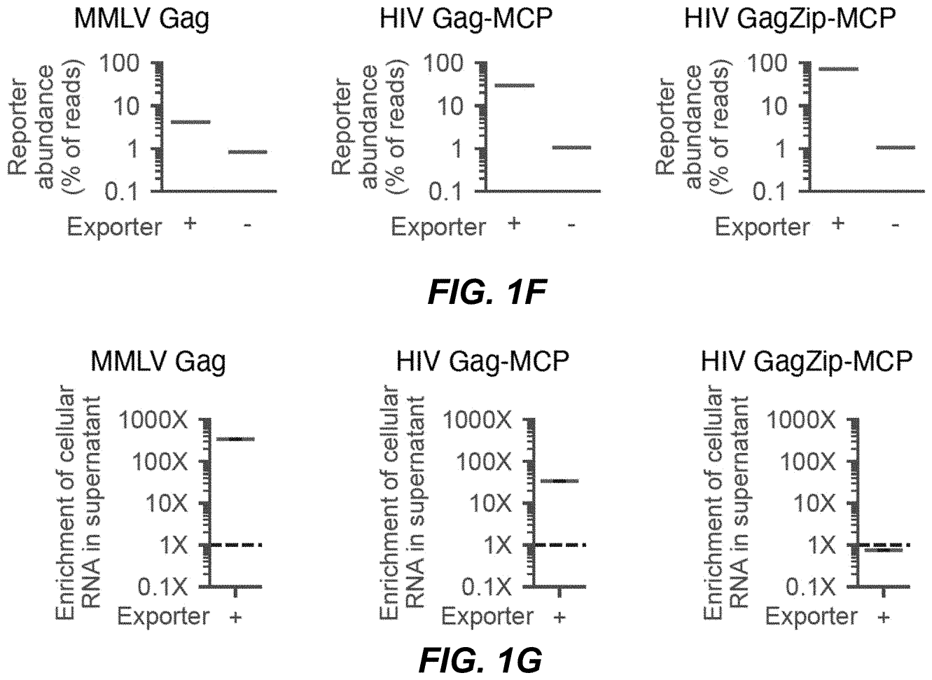

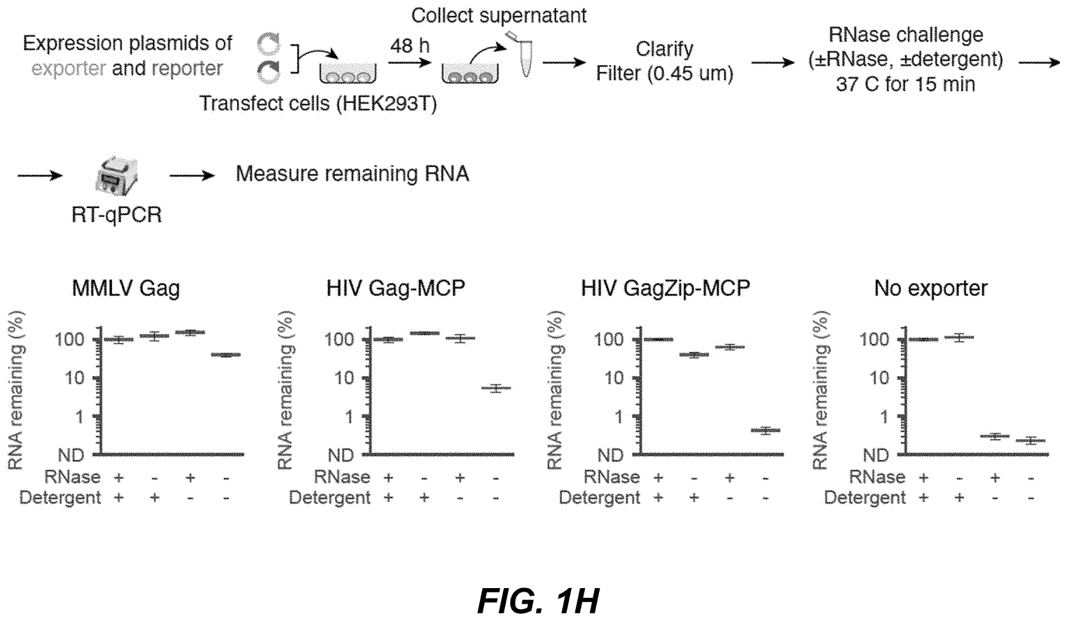

FIGS. 1A-1H depict non-limiting exemplary schematics and data related to engineered viral RNA exporters efficiently and specifically packaging, secreting, and protecting RNA. FIG. 1A depicts a non-limiting exemplary schematic of non-destructive measurement cell population and state dynamics. Sender cell exports RNA within genetically encoded nanoparticles. RNA sequence encodes information about the sender cell, such as its identity and state, which is read out by collecting and sequencing the exported RNA. Longitudinal samples directly measure cell population and state dynamics. FIG. 1B depicts non-limiting exemplary schematics of viral RNA exporter designs. Left, exporter based on Moloney Murine Leukemia Virus (MMLV) Gag capsid protein. MMLV Gag self-assembles to form viral capsid and packages RNA tagged with MMLV packaging signal (Psi) via interaction between MMLV Gag and Psi. Capsid is secreted from cell within a lipid envelope. Middle, exporter based on Human Immunodeficiency Virus (HIV) Gag capsid protein fused with RNA binding domain MS2 coat protein (MCP). HIV Gag domain self-assembles to form viral capsid. MCP binds RNA tagged with MS2 packaging signal, enabling specific packaging of target RNA. Right, exporter based on HIV Gag without nucleocapsid domain fused with leucine zipper dimerization domain from GCN4 (Zip), designated GagZip, which nucleates self-assembly via homodimerization of Zip to form viral capsid. RNA is specifically packaged via interaction of MS2 packaging signal with MCP. FIG. 1C depicts data related to negative stain transmission electron microscopy showing secretion of enveloped particles from HEK293T cells transfected with plasmids encoding RNA exporters. Scale bar, 100 nm. FIG. 1D depicts data related to the efficiency and specificity of RNA export to supernatant determined by reverse transcription followed by quantitative PCR (RT-qPCR). Top, schematic of experiment. HEK293T cells were transfected with expression plasmids of RNA exporter and reporter, the supernatant was collected after 48 hours, the supernatant was clarified by centrifugation at 3000 g for 5 minutes, the supernatant was passed through a 0.45 um filter, the RNA was extracted, and RT-qPCR was carried out to measure reporter RNA abundance in supernatant. Bottom, reporter RNA molecules detected in supernatant. Results indicate that viral RNA export systems efficiently release RNA from cells, and the specificity of release is improved by engineering specific RNA binding interactions into HIV Gag-MCP and GagZip-MCP. FIG. 1E depicts a non-limiting exemplary schematic of an experiment to characterize RNA exporters by sequencing. HEK293T cells were transfected with expression plasmids of RNA exporter and reporters with and without packaging signals, supernatant and cells were collected after 48 hours, the supernatant was clarified by centrifugation at 3000 g for 5 minutes, and the supernatant was passed through a 0.45 um filter. Then RNA standards were spiked into supernatant to enable determination of relative abundance of exported RNA across samples. RNA was extracted, libraries were prepared, and the RNA was sequenced. The reads from supernatant and cells were used to characterize export efficiency, specificity, and bias. The reads from cells were used to characterize transcriptional perturbations due to exporter expression. FIG. 1F depicts data related to the specificity of RNA export determined by sequencing. Improved packaging specificity of Gag-MCP and, to an even greater degree, GagZip-MCP results in a greater abundance of target reporter RNA among supernatant RNA, in comparison with MMLV Gag. This improvement may enhance the sensitivity of RNA export-based measurement systems. FIG. 1G depicts data related to the quantification of endogenous cellular RNA among supernatant RNA. Improved packaging specificity of Gag-MCP and, to an even greater degree, GagZip-MCP reduces the abundance of endogenous cellular RNA among supernatant RNA. FIG. 1H depicts data related to RNA exporters packaging and protecting RNA from degradation. Top, schematic of experiment to measure protection from RNase challenge. Bottom, RNA remaining after RNase challenge. Results show that RNA packaged and secreted by RNA exporters is protected from degradation, except in the presence of detergent, supporting a model of RNA export within lipid enveloped nanoparticles.

FIGS. 2A-2D depict non-limiting exemplary schematics and data related to the design and characterization of engineered viral RNA exporters. FIG. 2A depicts non-limiting exemplary domain architectures of viral RNA exporters. Left to right, N- to C-terminus. Colors indicate distinct protein domains. FIG. 2B depicts data related to the quantification of RNA export by viral RNA exporters with various architectures using reverse transcription followed by quantitative PCR (RT-qPCR). Cells were transfected with the indicated components of the RNA export system, supernatant was collected after 48 hours, RNA was extracted, and RT-qPCR was performed to measure the number of reporter RNA (mCherry mRNA) molecules in supernatant. Bars indicate mean of three technical replicates of qPCR, which are shown individually as dots. Colors indicate experimental condition, with grays showing key technical controls of omitting exporter or packaging signal. Results indicate that viral RNA export systems with a variety of protein architectures are capable of efficient and specific export of target RNA. Export can yield>1000-fold increased abundance of reporter RNA in supernatant, in comparison with no exporter and no packaging signal controls. FIG. 2C depicts data related to the quantification of RNA export system components in cellular RNA by RT-qPCR, using the same specimens as (B). Results indicate that expression of system components cannot explain the observed variation in RNA export efficiency in key technical controls, supporting that RNA export is exporter- and packaging signal-dependent. Dashed line indicates limit of detection, as determined by signal in control sample lacking DNA in transfection. FIG. 2D depicts data related to technical characteristics of RT-qPCR assay for RNA export to supernatant, indicating that assay faithfully and reproducibly measures RNA, not DNA, abundance. qPCR was performed with omission of key protocol steps: DNase treatment (labeled No DNase) or reverse transcription (labeled No RT). These conditions reveal that DNase treatment substantially reduces DNA contamination (which likely originates from transfected plasmid) to levels below the abundance of cDNA in samples lacking exporter. Thus, the assay faithfully measures RNA abundance within the relevant range for characterization of exporter performance. Assay was performed on three replicate wells, which were transfected independently and are indicated by different color dots, with three technical replicates of qPCR per well. Bars indicate mean of all replicates per condition. Consistency across replicate wells demonstrates that the assay is reproducible. Dashed line indicates limit of detection, as determined by signal in control sample lacking DNA in transfection.

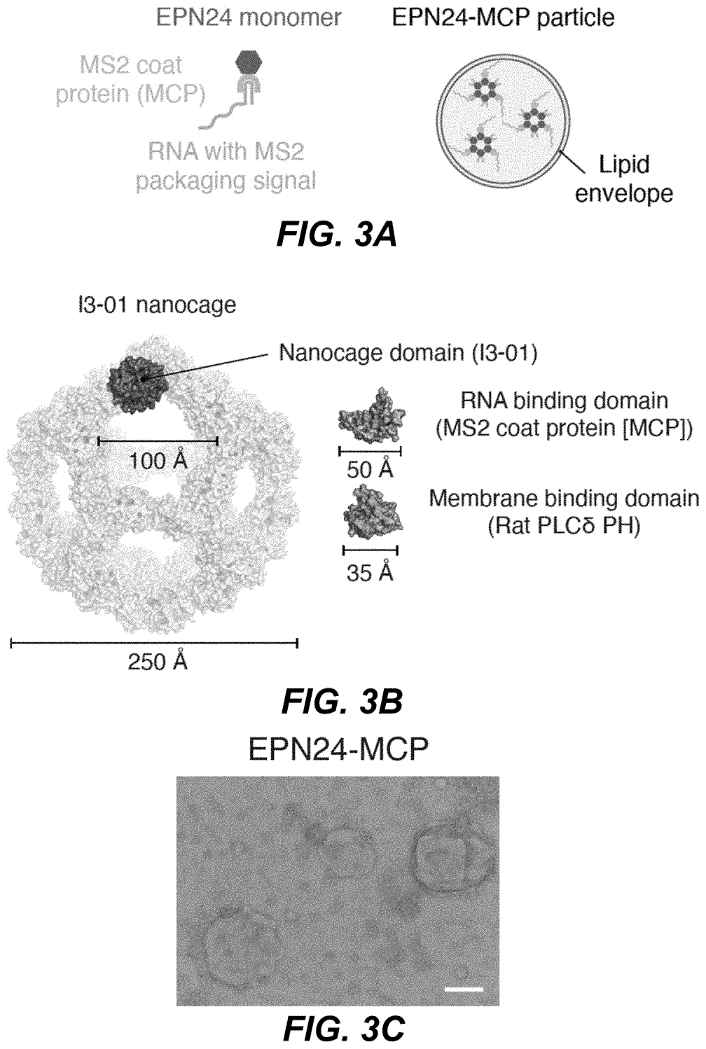

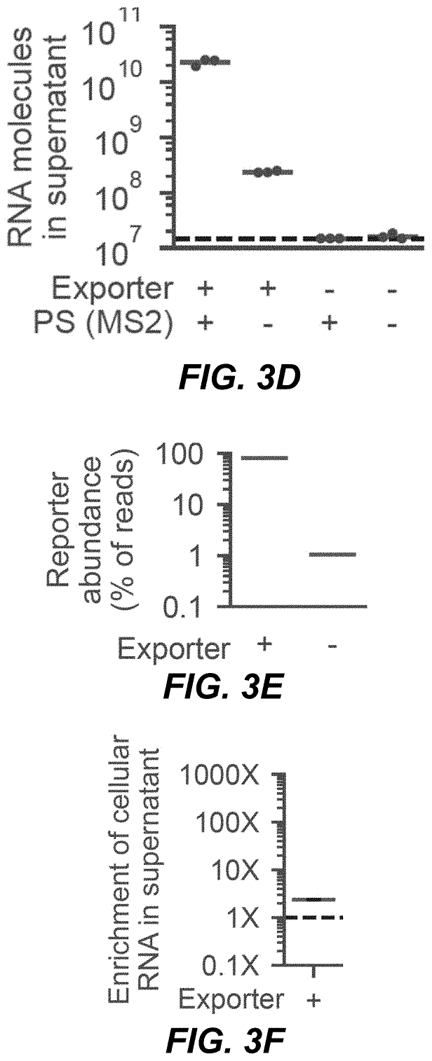

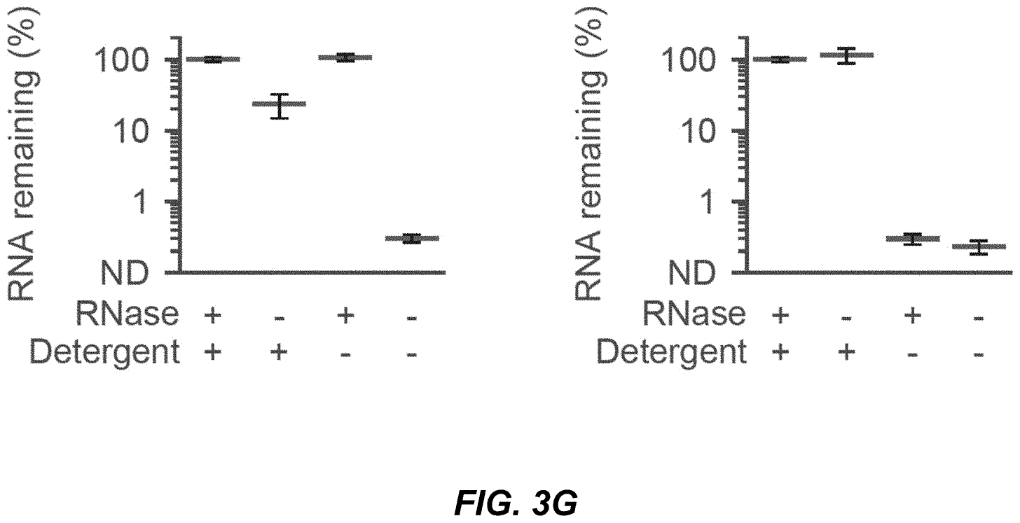

FIGS. 3A-3G depict non-limiting exemplary schematics and data related to synthetic non-viral RNA exporters efficiently and specifically packaging, secreting, and protecting RNA. FIG. 3A depicts a non-limiting exemplary schematic of synthetic non-viral RNA exporter design. Exporter comprises enveloped protein nanocage domain EPN24 fused with RNA binding domain MS2 coat protein (MCP). Exporter self-assembles to form nanocages, which package RNA tagged with MS2 packaging signal via interaction between MCP and MS2 aptamer. Exporter particles are composed of multiple nanocages secreted from the cell within an enveloped vesicle. FIG. 3B depicts a non-limiting exemplary protein design model of an RNA-exporting nanocage. Left, experimentally determined model of protein nanocage (PDB 5KP9). Monomer of 13-01 interaction domain is shown in purple. Right, x-ray crystal structures of MCP (PDB 1MSC) and membrane binding domain (rat phospholipase C delta Pleckstrin Homology domain, PLCD PH; PDB 1MAI), which are fused to 13-01 to form EPN24-MCP. Physical dimensions suggest that sufficient space exists within the 13-01 nanocage to accommodate the MCP and PLCD PH domains. FIG. 3C depicts data related to negative stain transmission electron microscopy showing secretion of enveloped particles from HEK293T cells transfected with plasmids encoding RNA exporter. Scale bar, 100 nm. FIG. 3D depicts data related to the efficiency and specificity of RNA export to supernatant determined by reverse transcription followed by quantitative PCR (RT-qPCR). Schematic of experiment is shown in FIG. 1D. Results indicate that synthetic RNA exporter EPN24-MCP releases reporter RNA from cells similarly efficiently and specifically as viral RNA exporters. FIG. 3E depicts data related to the specificity of RNA export determined by sequencing. Results show that the reporter RNA is the vast majority of exported RNA, indicating that RNA export is efficient and specific, similar to engineered viral RNA exporters. FIG. 3F depicts data related to the quantification of endogenous cellular RNA among supernatant RNA showing specific RNA export, similar to engineered viral RNA exporters. FIG. 3G depicts data related to the RNA exporters packaging and protecting RNA from degradation. Results show that RNA packaged and secreted by synthetic RNA exporters is protected from degradation, except in the presence of detergent, supporting a model of RNA export within lipid enveloped nanoparticles.

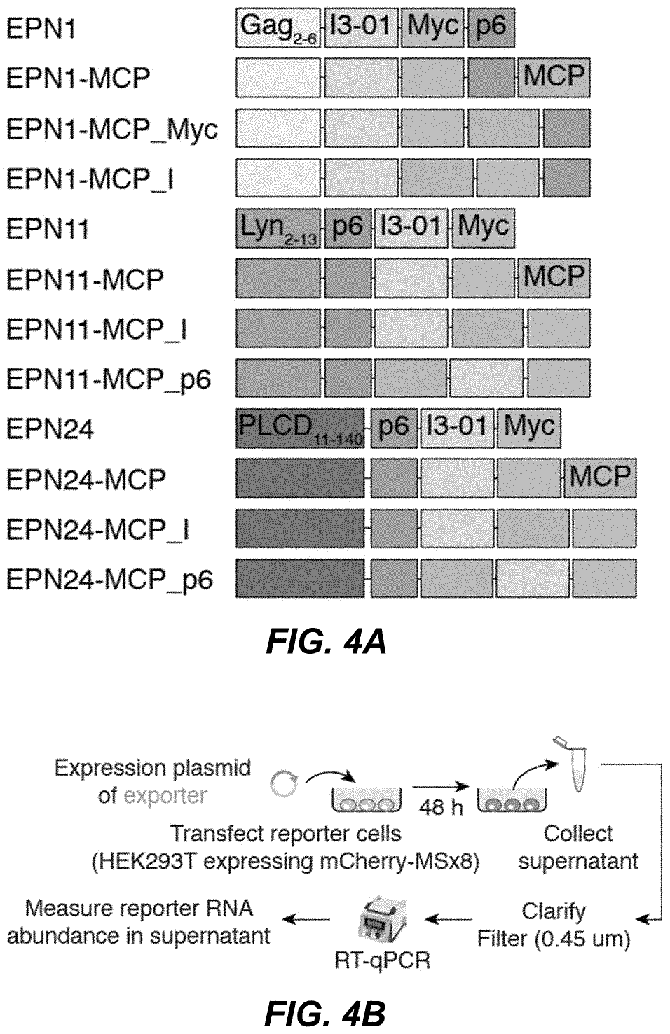

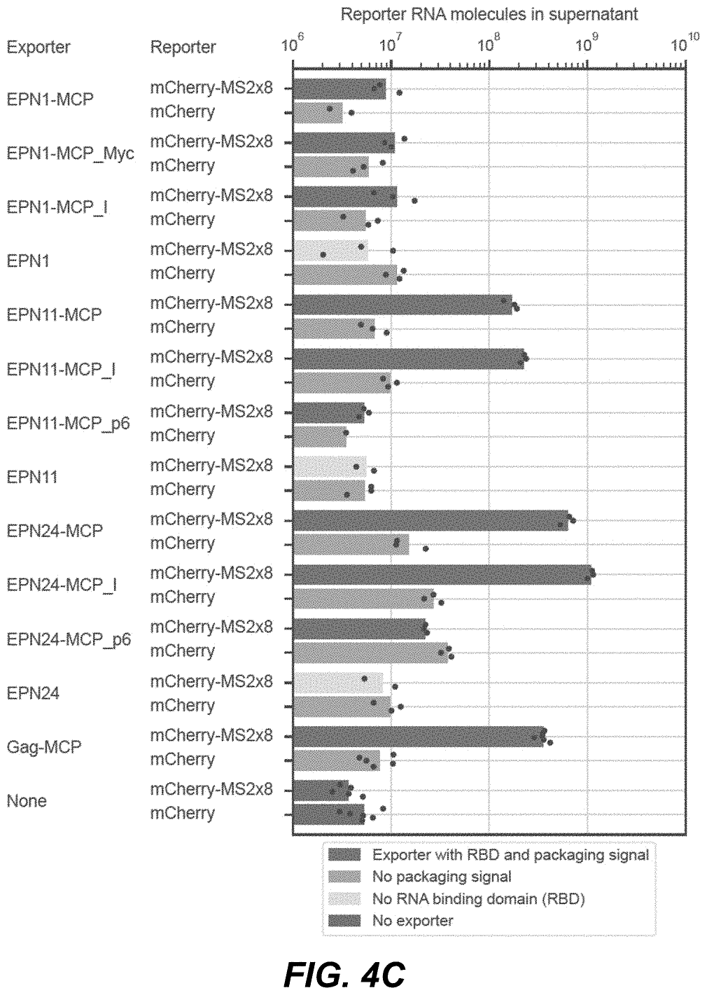

FIGS. 4A-4C depict non-limiting exemplary schematics and data related to the design and characterization of synthetic RNA exporters. FIG. 4A depicts non-limiting exemplary domain architectures of synthetic RNA exporters. Left to right, N- to C-terminus. Colors indicate distinct protein domains. Gag₂₋₆ is the myristoylation motif from HIV-1 NL4-3 Gag (amino acid (AA) residues 2-6). Lyn₂₋₁₃ is the myristoylation/palmitoylation motif from Lyn kinase (AA residues 2-13). PLCD₁₁₋₁₄₀ is the Pleckstrin Homology domain from rat phospholipase Cδ. p6 is the p6 domain from HIV-1 NL4-3 Gag. MCP is the MS2 coat protein, which serves as an RNA binding domain. MCP was fused in various positions within the RNA exporter sequence. FIG. 4B depicts a non-limiting exemplary schematic of assay for RNA export using stable reporter cell line. Lentivirus was used to create stable reporter cell lines that constitutively express mCherry either with or without the MS2 packaging signal, consisting of a tandem array of eight MS2 aptamers in the 3′ untranslated region (3′ UTR) of the mCherry transcript. To perform the assay, an expression plasmid of the RNA exporter was transfected into reporter cells, supernatant was collected after 48 hours, the supernatant was clarified by centrifugation at 3000 g for 5 minutes, the supernatant was passed through a 0.45 um filter, RNA was extracted, and RT-qPCR was carried out to measure reporter RNA abundance in supernatant. FIG. 4C depicts data related to the quantification of RNA export by synthetic RNA exporters with various architectures using RT-qPCR. Bars indicate mean of three technical replicates of qPCR, which are shown individually as dots. Colors indicate experimental condition, with grays showing key technical controls of omitting exporter, RNA binding domain, or packaging signal. Results indicate that synthetic RNA exporters with various architectures are capable of efficient and specific export of RNA. Export yields similarly increased abundance of reporter RNA in supernatant compared with the viral exporter Gag-MCP, suggesting similar export efficiency.

FIGS. 5A-5E depict data related to physical and functional features of RNA export systems. FIG. 5A depicts data related to the quantification of RNA export by RT-qPCR revealing that the rate of RNA export depends on copy number of the packaging signal, which is a tandem array of MS2 aptamers. Cells were transfected with RNA export system components, including reporters with varying numbers of MS2 aptamers within a tandem repeat array in the 3′ UTR, supernatant was collected, and exported RNA was measured using RT-qPCR, as shown in FIG. 1D. Results show that the rate of RNA export increases with the copy number of MS2 aptamers, and saturates at 8 copies. Dashed line indicates limit of detection, as determined by signal in control sample lacking DNA in transfection. FIG. 5B depicts data related to the quantification of RNA export by RT-qPCR reveals that RNA exporters can package their own RNA. An RNA export system was created that packages its own transcript by incorporating the packaging signal, consisting of a tandem array of eight MS2 aptamers, into the 3′ UTR of the RNA exporter transcript itself. Cells were transfected with this single RNA export system component, which serves as its own reporter, collected supernatant, and exported RNA was measured using RT-qPCR targeting GFP. Results indicate that the RNA exporter Gag-MCP efficiently exports its own transcript, if its transcript contains the packaging signal. Dashed line indicates limit of detection, as determined by signal in control sample lacking DNA in transfection. FIG. 5C depicts data related to negative stain transmission electron microscopy showing the lack of enveloped particles in supernatant collected from HEK293T cells transfected with plasmids encoding the reporter, but not the exporter. Scale bar, 100 nm. FIG. 5D depicts data related to the physical size of RNA export particles, as determined by dynamic light scattering. FIG. 5E depicts data related to the quantification of RNA export by RT-qPCR from mouse embryonic stem cells (mESCs). Species-chimeric viral RNA exporters were designed that are adapted to mouse by replacing the matrix domain (MA) from HIV Gag with MA domain from MMLV, then fusing the RNA binding domain MCP, designated Gag_MHIV_MA-MCP (top). mESCs were transfected with this chimeric RNA exporter and other components of the RNA export system and quantified RNA export (bottom). This revealed that this engineered viral species chimera can export RNA in mESCs, indicating that RNA exporters can be adapted to a broad range of cell types and species.

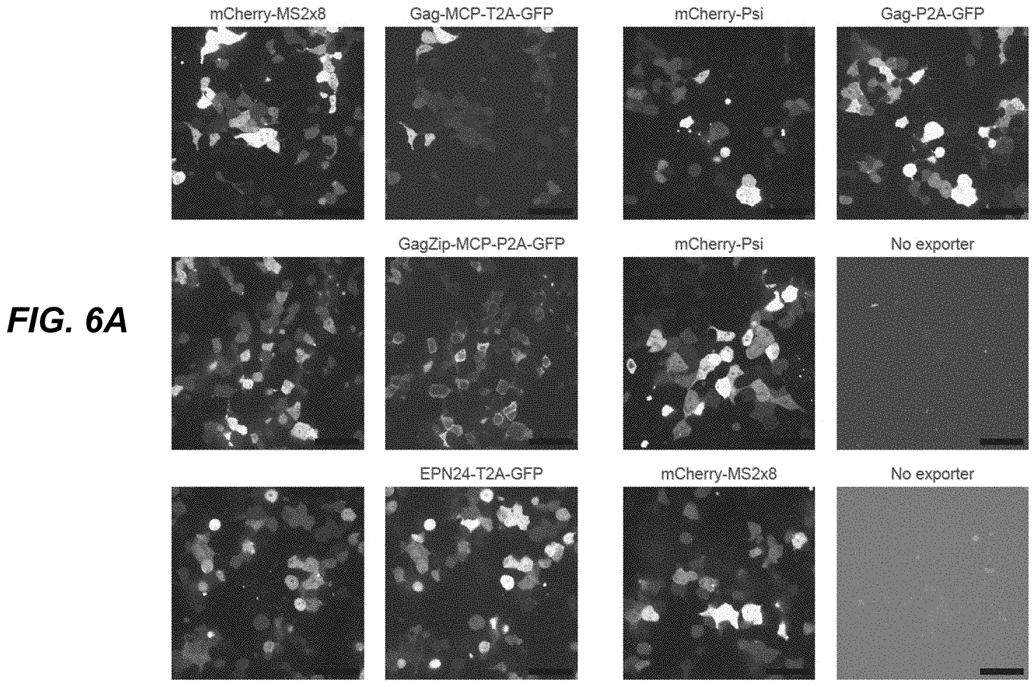

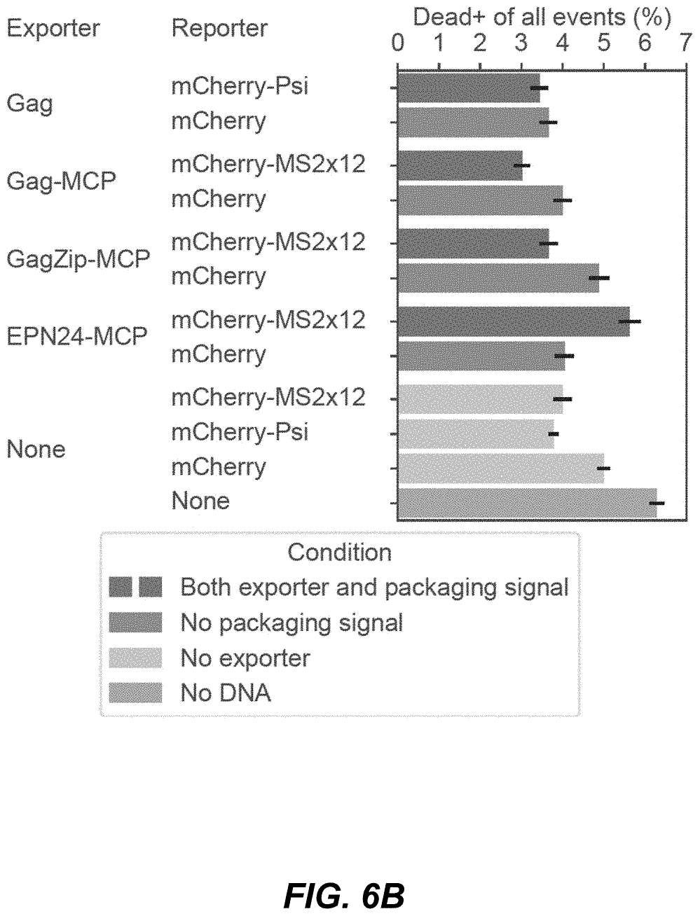

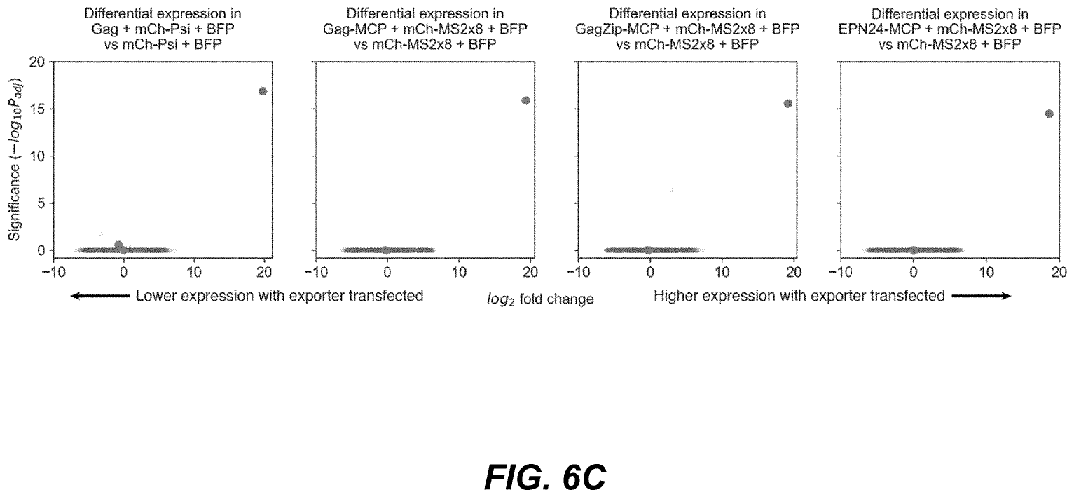

FIGS. 6A-6C depict data showing RNA exporters are non-toxic and do not perturb cellular morphology and transcriptome. FIG. 6A depicts non-limiting exemplary images of HEK293T cells transfected with RNA exporters and reporters. Scale bar, 50 um. Images reveal no apparent differences in cellular morphology between cells expressing RNA exporters and those that are not. FIG. 6B depicts data related to the quantification of toxicity of RNA exporter expression in HEK293T cells using flow cytometry with dead stain (ethidium homodimer-1). Results show that RNA exporter expression is non-toxic. Error bars show 95% confidence interval based on binomial sampling. FIG. 6C depicts data related to differential expression analysis of cellular transcriptomes of cells transfected with and without exporters. Each dot is a gene. Transgenes are shown in color. Results indicate that no genes are significantly differentially expressed in association with exporter expression, indicating that exporters do not detectably perturb cellular transcriptomes.

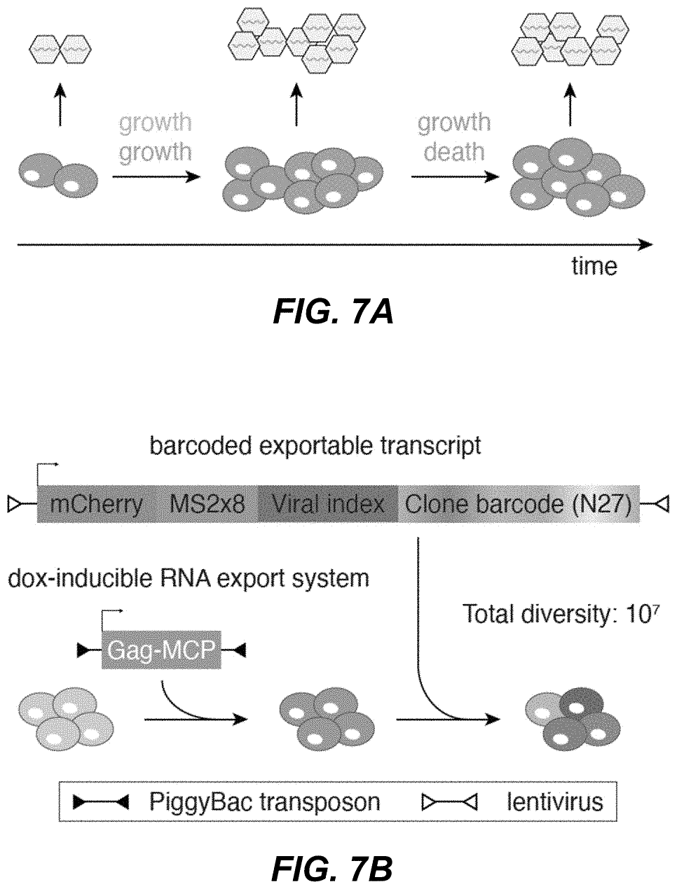

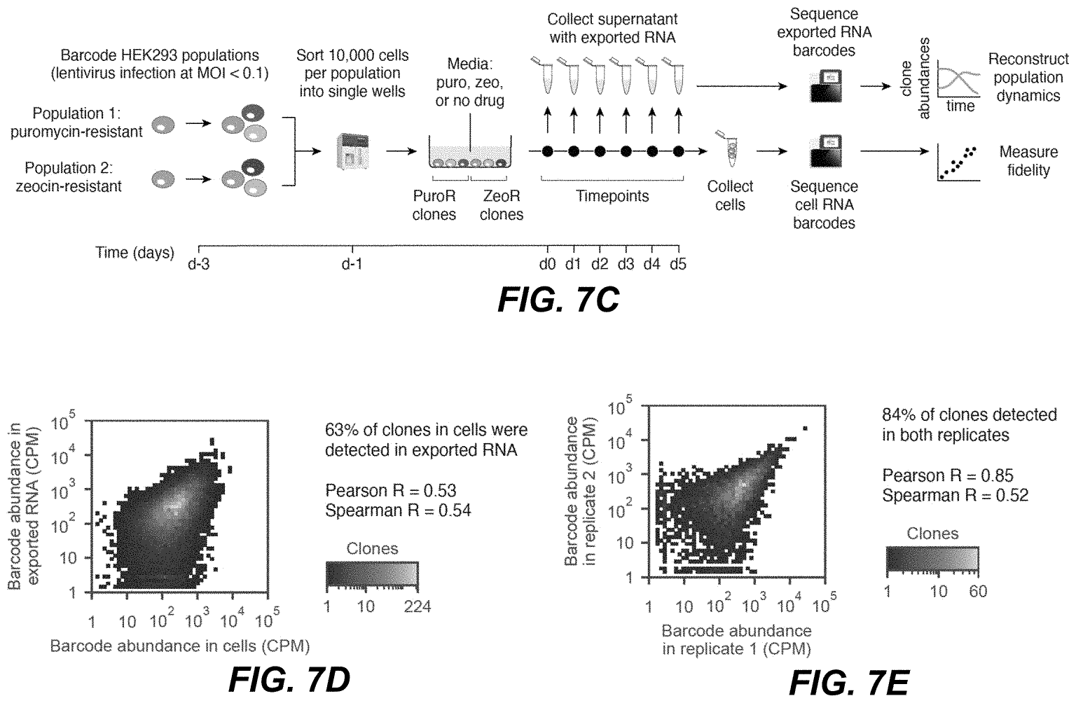

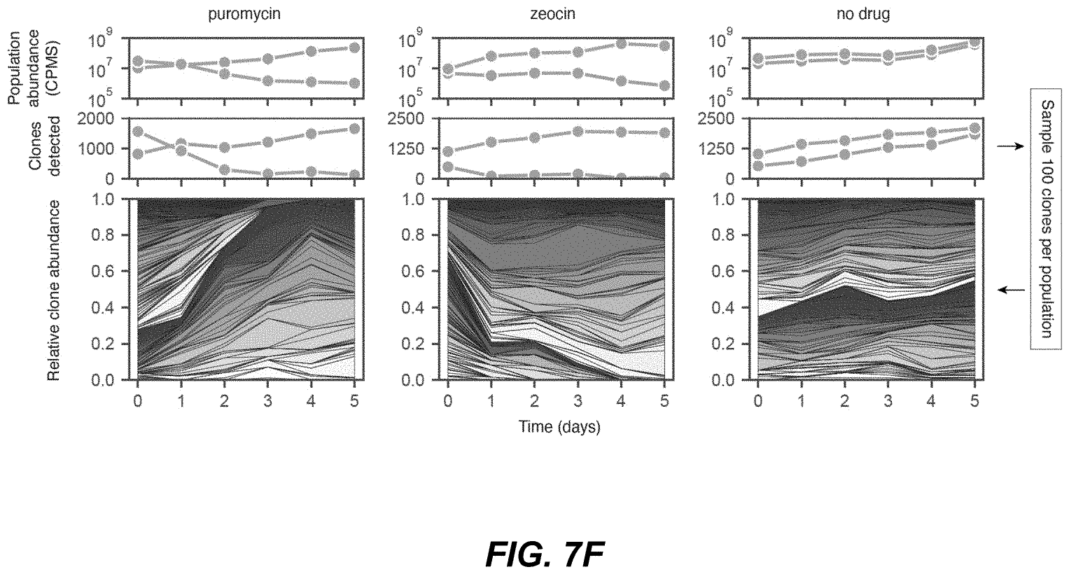

FIGS. 7A-7F depict non-limiting exemplary schematics and data related to RNA export enabling continuous monitoring of mammalian cell population dynamics. FIG. 7A depicts a non-limiting exemplary schematic of monitoring cell population dynamics using exported RNA barcodes. Each cell population is marked with a different sequence barcode, indicated by color. These barcodes are transcribed to RNA and exported. Abundance of the RNA barcodes reflects the sizes of the cell populations. FIG. 7B depicts a non-limiting exemplary design and construction of exportable RNA clone barcodes. HEK293 cells were stably transfected with doxycycline (dox)-inducible RNA export system (Gag-MCP) via PiggyBac transposon. Next, a diverse library of clone barcodes was delivered via lentivirus. These barcodes are constitutively transcribed into mRNA encoding the mCherry fluorescent protein, a tandem array of eight MS2 aptamers, a viral index barcode, and the clone barcode. FIG. 7C depicts a non-limiting exemplary schematic of an experiment to measure population dynamics. Two HEK293 populations were prepared with resistance to either puromycin or zeocin. These populations were barcoded by lentiviral infection of diverse clone barcode libraries with distinct viral indices, as shown in panel B. 5,000 cells of each population were sorted into a single well of a 48-well cell culture plate, and were cultured in media containing puromycin (puro), zeocin (zeo), or no drug. Supernatant was collected at daily timepoints. At the final timepoint, the cells were collected. RNA was extracted, libraries were prepared, and barcodes from supernatant and cells were sequenced. These barcode abundances were used to reconstruct clonal population dynamics, and compared the barcode abundances measured in supernatant and cells to determine the fidelity of the exported RNA reporter system. FIG. 7D depicts data related to the fidelity of clone abundance measured by exported RNA reporter system. A comparison was performed of the abundances of barcodes measured in cells, which serves as a “ground truth”, and supernatant. Strong correlation between the two abundances indicates that the measurement has high fidelity. FIG. 7E depicts data related to the reproducibility of clone abundance measured by exported RNA reporter system. The collected supernatant was split into two technical replicate samples, then measured and compared barcode abundances in each. Strong correlation between replicates indicates that the measurement is reproducible. FIG. 7F depicts data related to clone population dynamics revealed by exported RNA reporter system. Top, total abundance of each barcoded population, with puromycin-resistant shown in purple and zeocin-resistant shown in green. Middle, clones detected within each barcoded population. Bottom, relative abundance of 100 clones sampled at random from each population. Left, well containing puromycin; middle, well containing zeocin; right, well containing no drug. CPMS, counts per million of standard, which was spiked in prior to RNA extraction to enable normalization and quantitative comparison across samples.

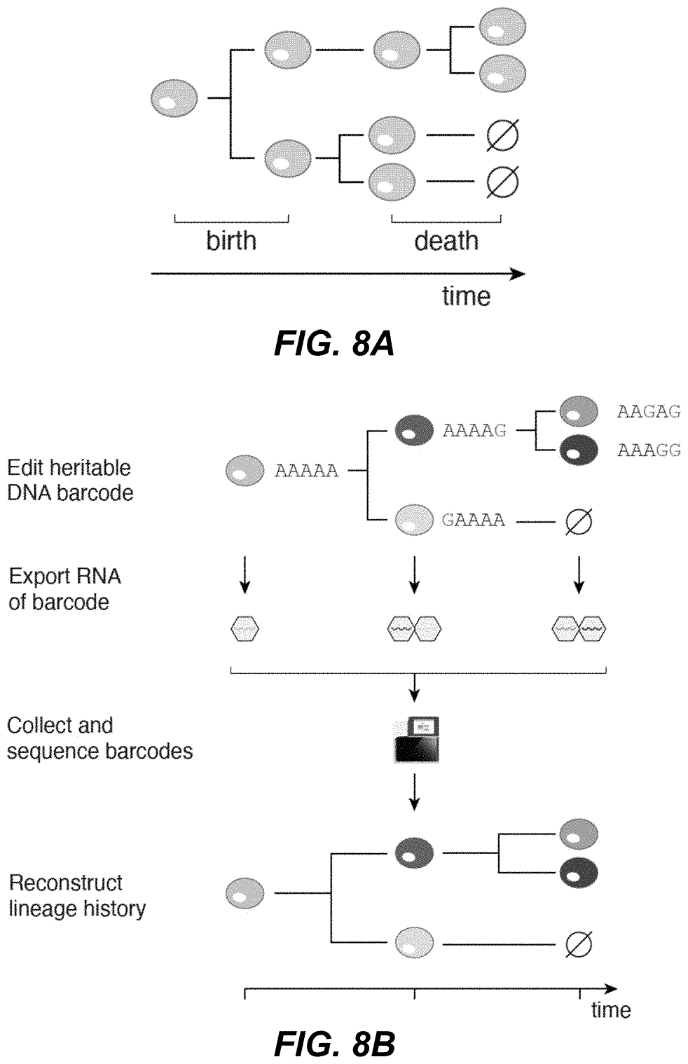

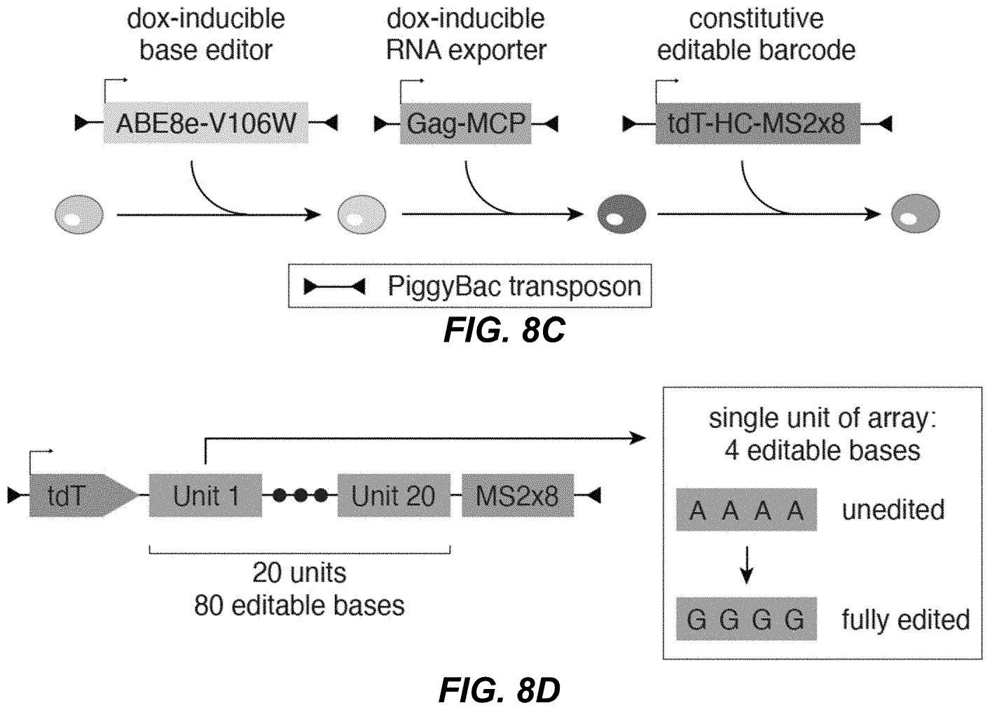

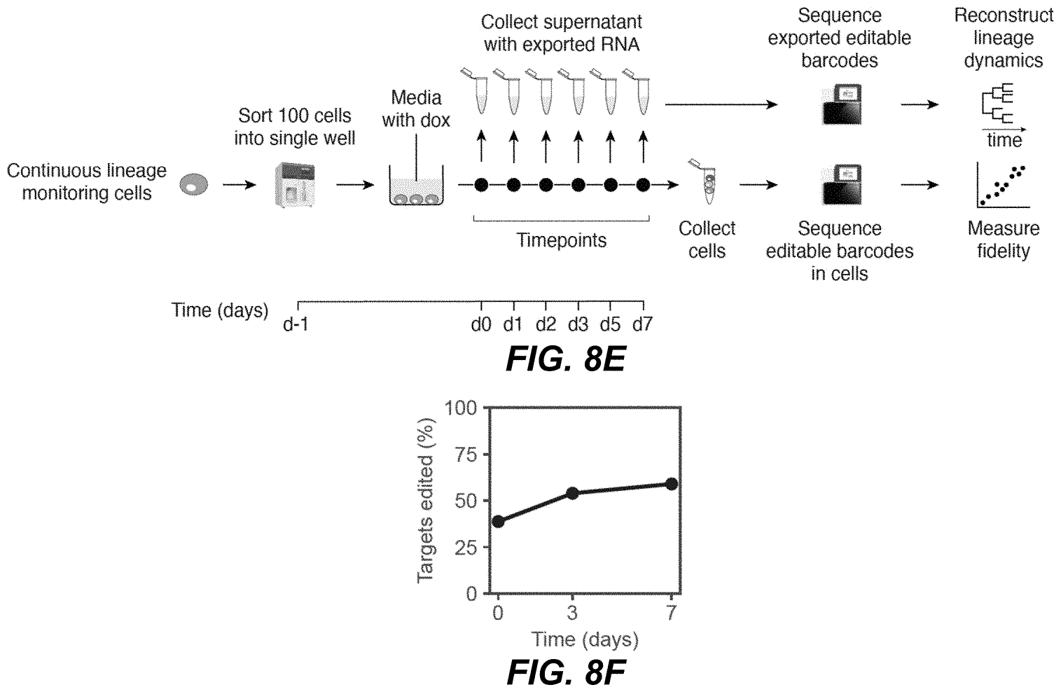

FIGS. 8A-8F depict non-limiting exemplary schematics and data related to RNA export enabling continuous monitoring of mammalian cell lineage dynamics. FIG. 8A depicts a non-limiting exemplary schematic of cell lineage illustrating changes across time, including birth and death events, and that measurements at the very end of an experiment (endpoint) can yield incomplete lineage histories. FIG. 8B depicts a non-limiting exemplary schematic of using RNA export of editable barcodes to continuously monitor cell lineage history. Cells undergo birth and death while editing a heritable DNA barcode (top), and transcribe and export the barcode (middle top). The barcodes were collected and sequenced (middle bottom) and used to reconstruct lineage history (bottom). The exported barcodes are collected without destroying the cells at various timepoints, enabling direct determination of cell lineage dynamics, and detection of lineages which do not exist at the endpoint. FIG. 8C depicts a non-limiting exemplary schematic of construction of cell lines for edit and export of lineage barcodes. HEK293 cells were stably transfected via PiggyBac transposon with doxycycline (dox)-inducible DNA base editor (ABE8e-V106W), dox-inducible RNA exporter (Gag-MCP), and constitutively transcribed editable barcodes (tdT-HC-MS2×8). Barcode transcripts contain a tandem array of eight MS2 aptamers, enabling export. FIG. 8D depicts a non-limiting exemplary design of editable exportable barcode. Editable barcode consists of 20 tandem copies of an editable unit, which itself has 4 bases that can be converted from A to G by the base editor. This barcode is transcribed in the 3′ UTR of a transcript encoding the fluorescent protein tdTomato, together with a tandem array of eight MS2 aptamers, enabling export. FIG. 8E depicts a non-limiting exemplary schematic of an experiment to monitor cell lineage dynamics. After constructing the cell lines, described in panel C, 100 cells were sorted into a single well of a 96-well cell culture plate, and cultured for 7 days. Supernatant was collected at various timepoints. At the final timepoint, the cells were collected. RNA was extracted, libraries were prepared, and barcodes from supernatant and cells were sequenced. Using these barcode sequences, phylogenetic reconstruction was performed to determine lineage dynamics. A comparison was performed of the barcodes detected in supernatant and cells to determine the fidelity of the exported RNA reporter system. FIG. 8F depicts data related to the quantification of lineage barcode editing across time. The fraction of editable bases converted from A to G within barcode sequencing reads obtained from supernatant increases through time, demonstrating progressive editing and export of barcodes.

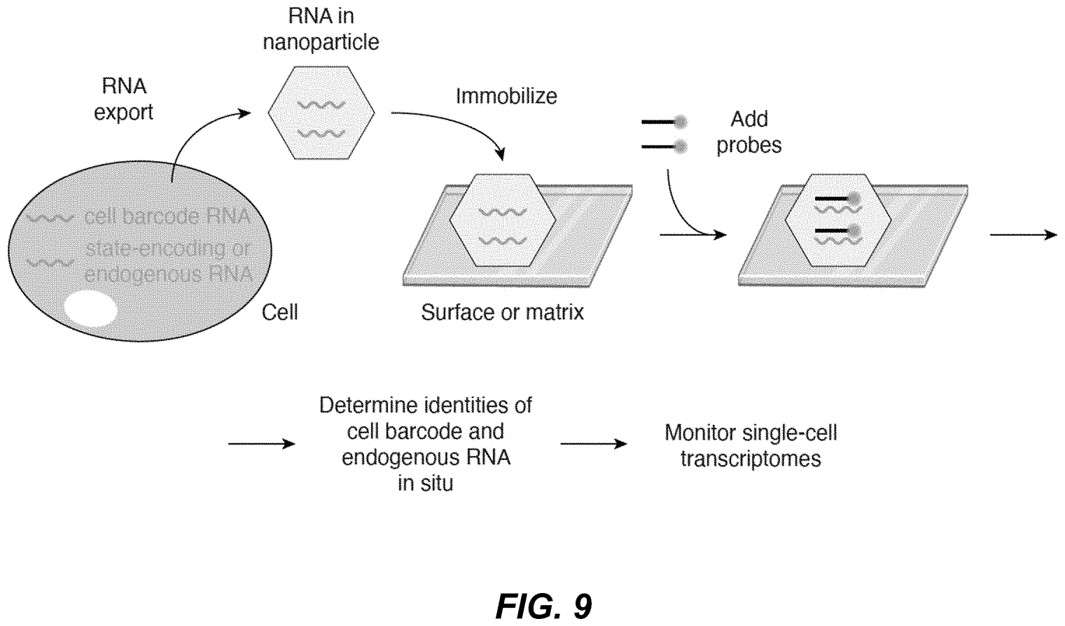

FIG. 9 depicts a non-limiting exemplary schematic of a technique to non-destructively monitor single-cell transcriptome dynamics using export of RNA. Cell packages and secretes RNA within nanoparticle. Importantly, the nanoparticle co-packages at least two types of transcript: (1) cell barcode RNA, which harbors a sequence barcode that uniquely identifies the cell of origin, and (2) state-encoding RNA, which reflects the state of the cell of origin. The state-encoding RNA could be an endogenous RNA, an engineered RNA whose abundance reflects the state of the cell, or an RNA which becomes chemically modified in a way that reflects the state of the cell. The RNA-containing nanoparticle is immobilized on a surface. The identifies of the RNA within the particle are determined using in situ measurement, such as fluorescence in situ hybridization or in situ sequencing, linking the cell barcode to the state of the cell of origin. By collecting and analyzing samples of exported RNA at various timepoints, single-cell transcriptomes can be dynamically monitored.

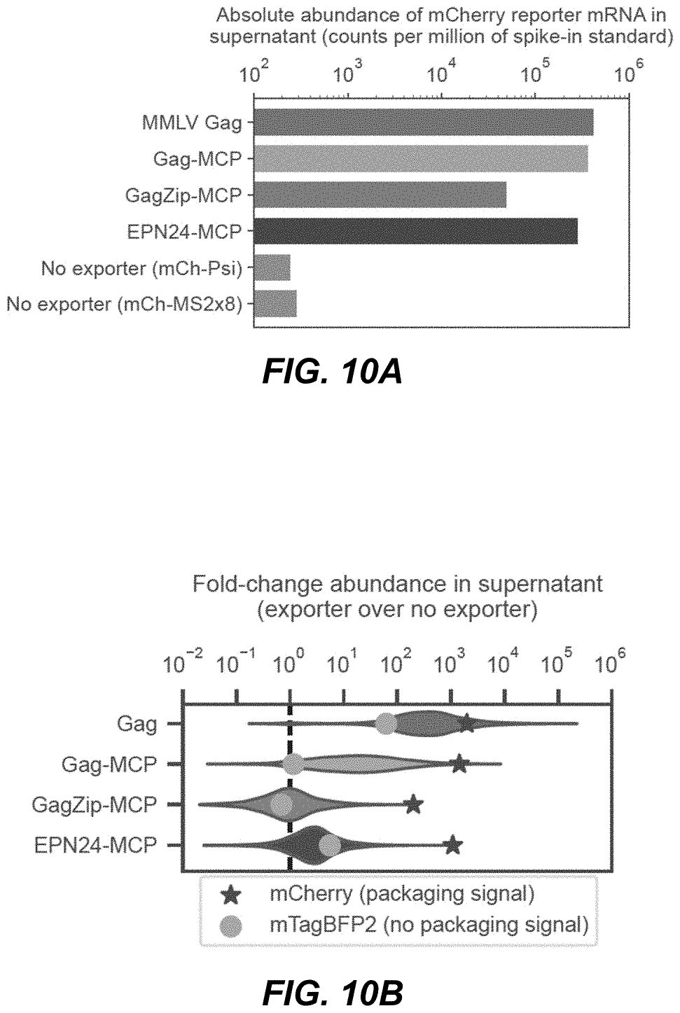

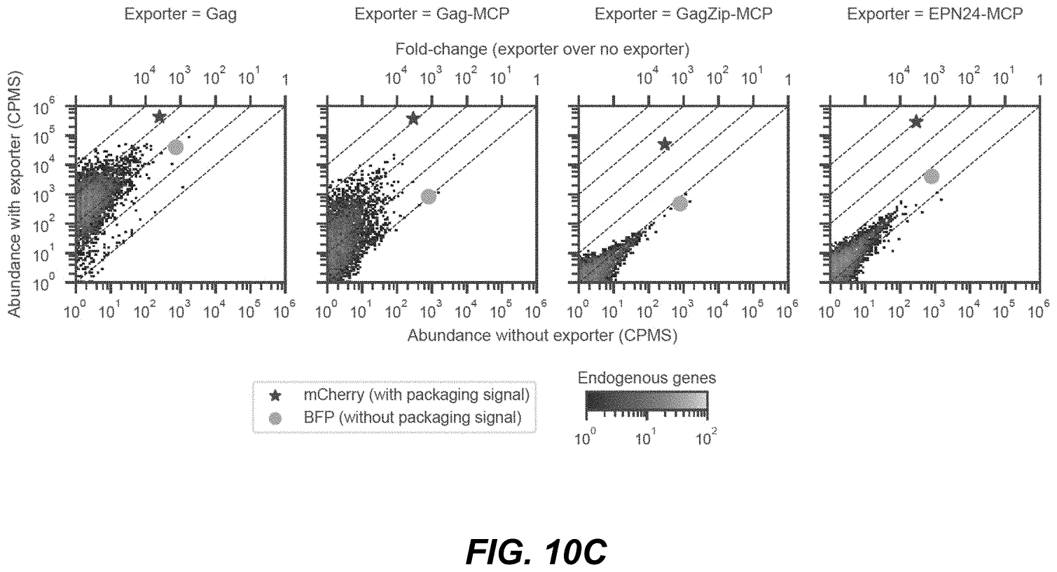

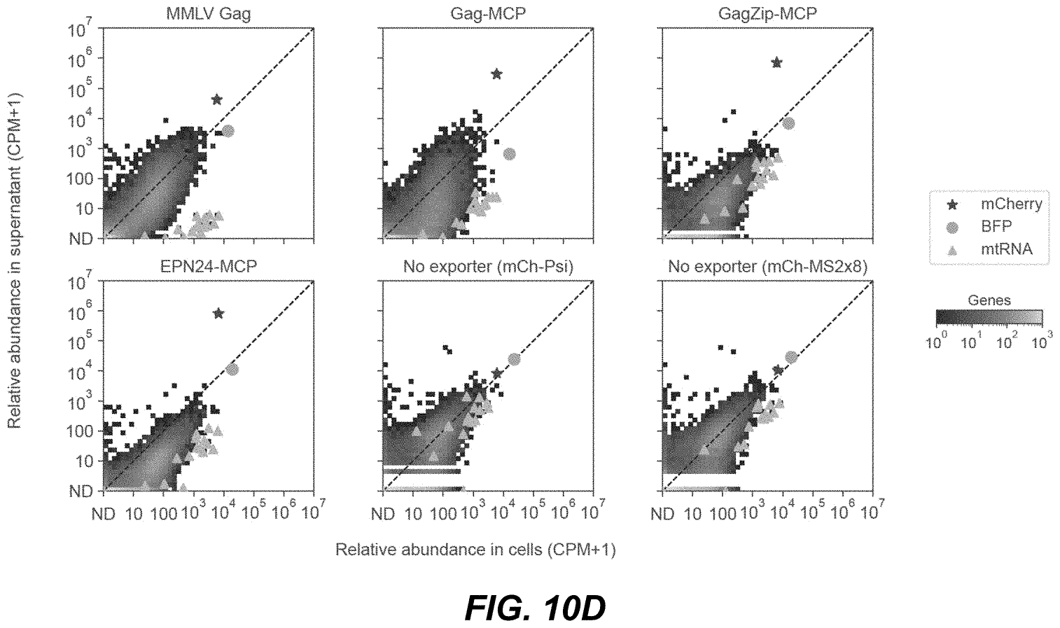

FIGS. 10A-10D depict data related to transcriptomic characterization of RNA export. To characterize the spectrum of RNA export, RNA sequencing of poly-adenylated transcripts from supernatant and cells was performed as shown in FIG. 1E. FIG. 10A depicts data related to the quantification of the absolute abundance of reporter RNA in supernatant. Using spike-in standards for calibration, the relative abundance of reporter RNA was determined, which contains the packaging signal, in supernatant of cells transfected with various exporters. Results indicate that the absolute abundance of exported RNA in supernatant is ˜1000-fold higher with RNA exporter compared to without exporter, indicating that export is efficient. FIG. 10B depicts data related to the quantification of the abundance of reporter and endogenous genes in supernatant of cells transfected with various exporters. Abundances are shown as fold-change compared with a comparable experimental condition lacking the exporter. Violins indicate the distribution of fold-changes for endogenous cellular genes. Symbols indicate the fold-changes of transgenic reporters, having (star) or lacking (circle) packaging signals. Dashed line indicates no change in abundance. FIG. 10C depicts data related to the abundance of endogenous genes and transgenes in supernatant of cells transfected with and without exporter. Top axis indicates fold-change between exporter and no exporter conditions. Results show that engineering improves RNA exporter specificity and reduces transcriptome-wide export. CPMS, counts per million of standard. FIG. 10D depicts data related to the relative abundance of endogenous genes and transgenes in RNA from cells and supernatant. Results indicate that exporters secrete an unbiased sample of cellular transcriptome, with the notable exception of mitochondrial RNA (mtRNA), which are predicted to be localized within mitochondria and thus not accessible for packaging to exporters assembling within the cytoplasm. CPM, counts per million. ND, not detected.

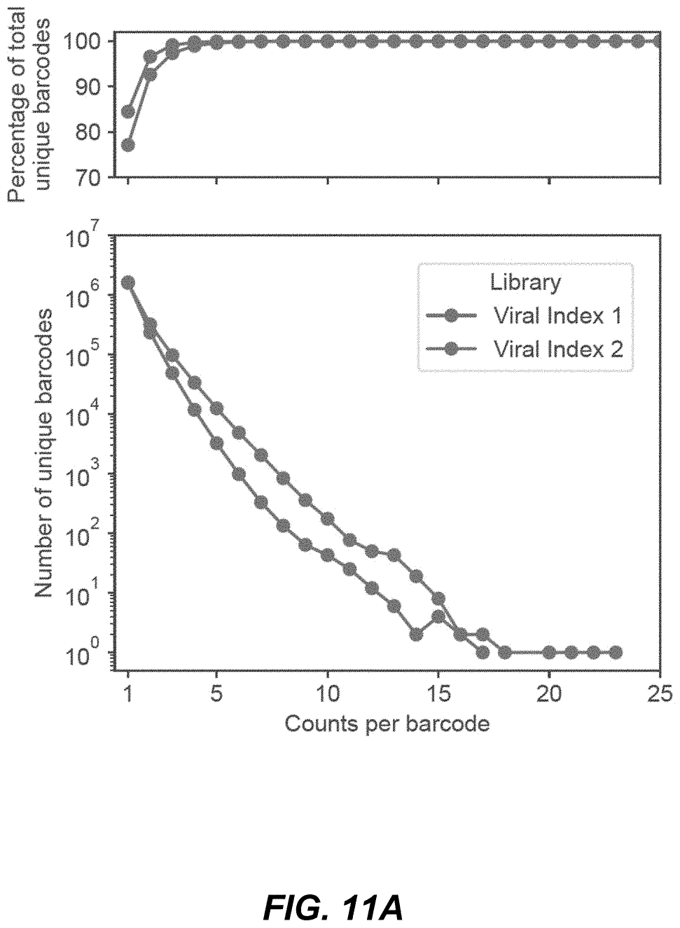

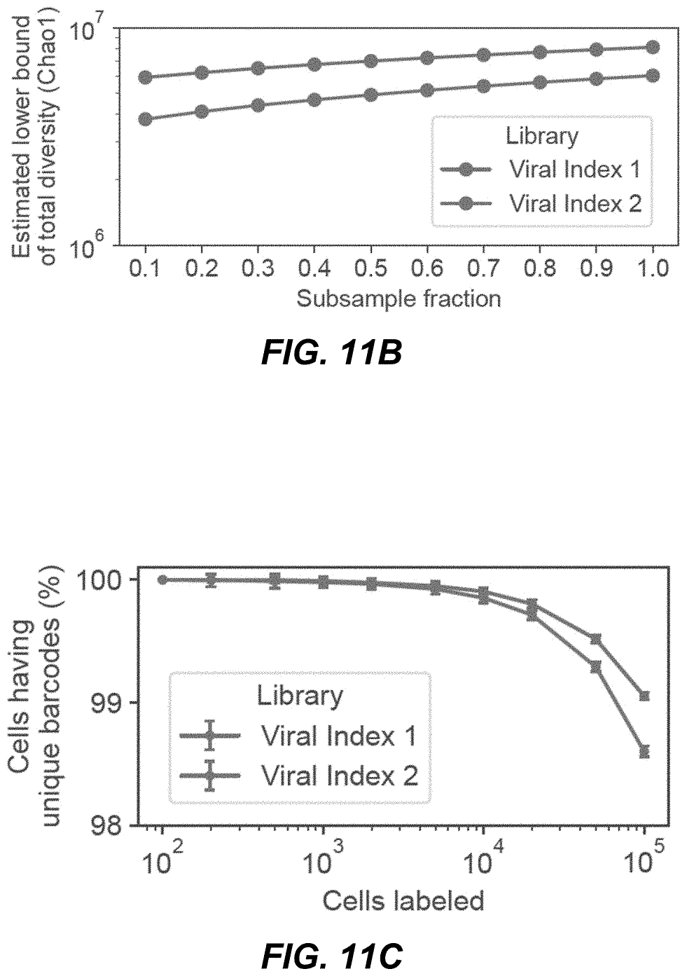

FIGS. 11A-11C depict data related to characteristics of barcode library used for clone labeling. Viral barcode libraries were prepared by cloning and sequenced to characterize their diversity. Two such libraries were prepared having distinct viral indexes. FIG. 11A depicts a non-limiting exemplary graph related to diversity of barcodes. FIG. 11B depicts a non-limiting exemplary graph related to estimation of total diversity of barcode libraries based on capture-recapture statistics. FIG. 11C depicts a non-limiting exemplary graph related to labeling capacity of barcode libraries based on the collision rate within samples of varying size.

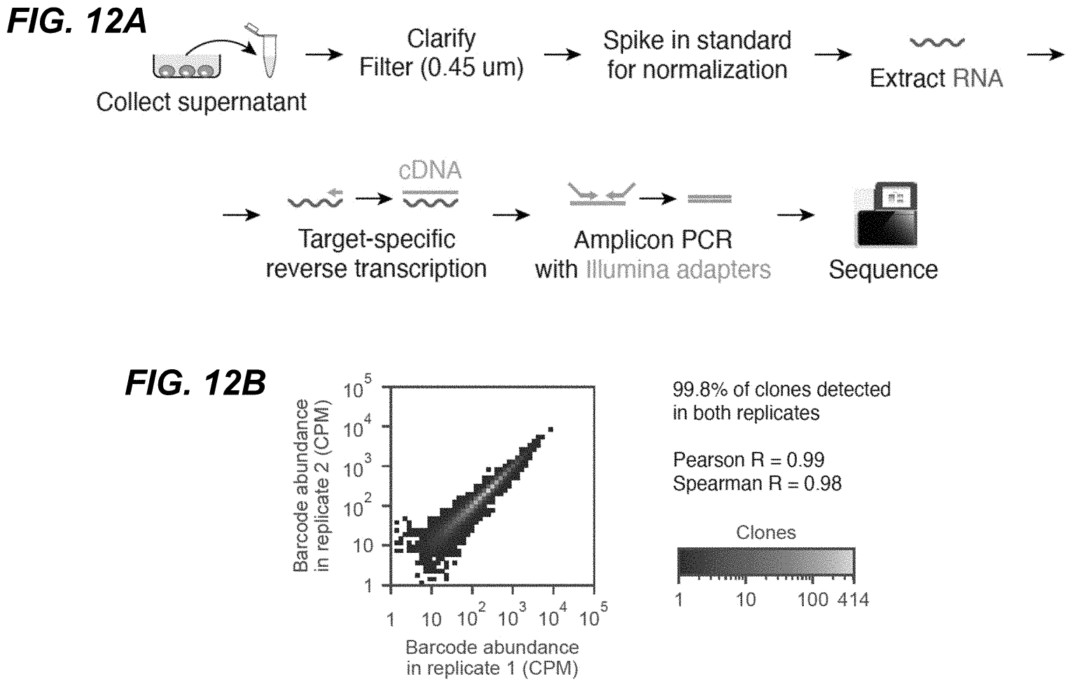

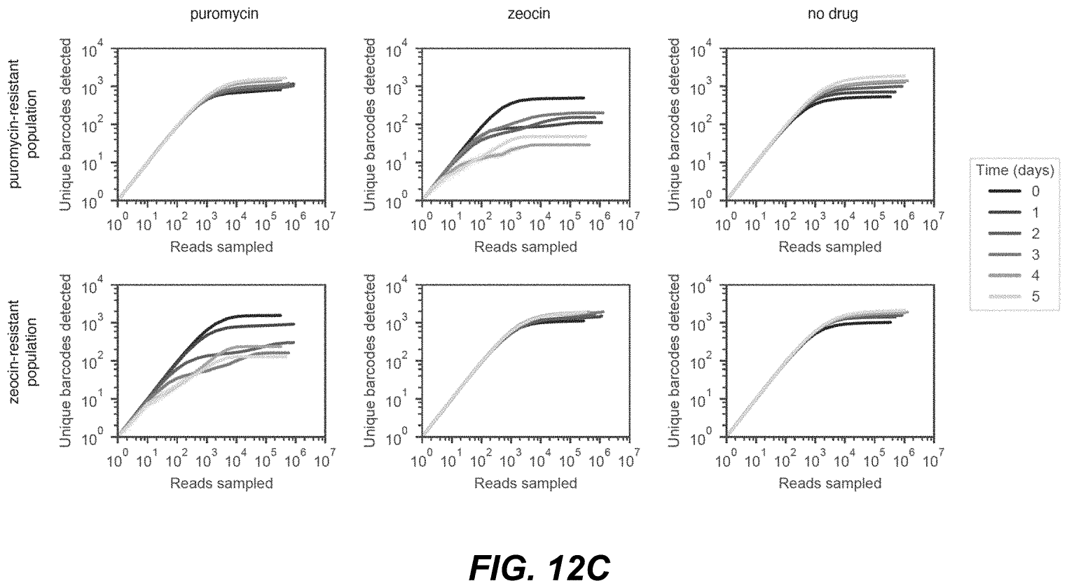

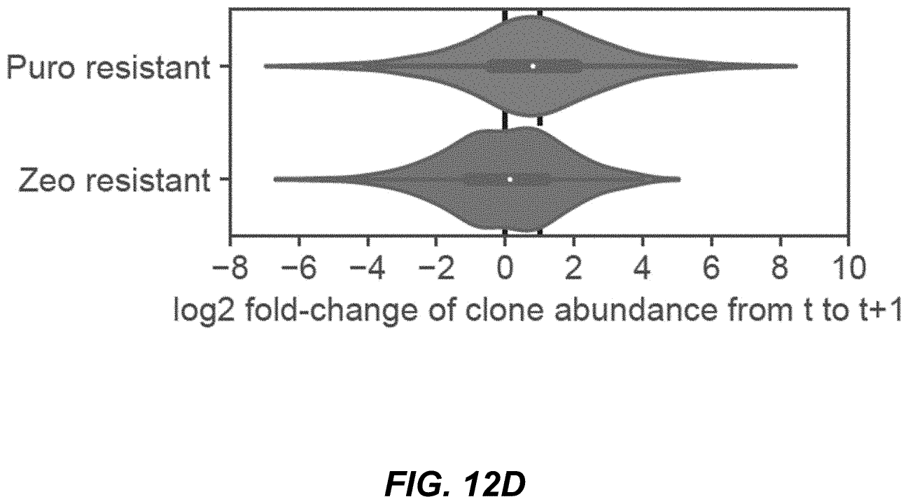

FIGS. 12A-12D depict non-limiting exemplary schematics and data related to characteristics and performance of measurement of clone dynamics using RNA export and sequencing. FIG. 12A depicts a non-limiting exemplary schematic of experimental workflow for measuring clone abundances by RNA export and sequencing. Supernatant was collected, the supernatant was clarified by centrifugation at 3000 g for 5 minutes, the supernatant was passed through a 0.45 um filter, an RNA standard was spiked in to enable comparison of abundance across samples, and RNA was extracted. Then reverse transcription was carried out with target-specific primers binding to the reporter RNA, and polymerase chain reaction (PCR) with primers flanking the reporter barcode region. PCR primers also included sequencing adapters, enabling one-step library preparation. Amplicons were sequenced using Illumina platform. FIG. 12B depicts data related to the quantification of clone abundances in replicate samples of cellular RNA. FIG. 12C depicts data related to rarefaction analysis of sequencing depth. Results show that clone discovery is saturated at <1M reads/sample. Related to experiment shown in FIG. 7F. FIG. 12D depicts data related to distributions of clone growth rates based on changes in abundance from one timepoint (t) to the next timepoint (t+1) in well containing puromycin. Left dashed line indicates no change, right dashed line indicates two-fold change. Results indicate that puromycin-resistant clones approximately double in abundance in 24 hours, in line with their doubling time observed by microscopy, whereas zeocin-resistant clones do not grow, due to lack of resistance to puromycin. Related to experiment shown in FIG. 7F.

FIG. 13 depicts a non-limiting exemplary schematic of the methods, compositions, and systems provided herein. An RNA reporter (e.g. mCherry-MSx12, consisting of mRNA encoding mCherry with twelve tandem repeats of the MS2 stem-loop aptamer in the 3′ untranslated region) is transcribed in a living cell, exported to the outside of the cell by an RNA exporter (e.g. Gag-MCP, consisting of HIV Gag protein fused to MS2 coat protein), collected, and sequenced or analyzed. Information about the state of the cell is encoded in the sequence of the RNA reporter. The exported RNA reporter includes a barcode which uniquely identifies the cell from which the RNA originated. To provide a cell barcode that is unique despite the heritability of genomically integrated barcodes during cell division, some embodiments provided herein include the ability to edit the barcode using an editor (e.g. adenine base editor ABEmax), giving rise to distinct barcodes among descendant cells after division.

FIG. 14 depicts a non-limiting schematic related to measurement of cell barcodes by sequencing of exported RNA reporter. HEK293T cells were transiently transfected with plasmid DNA containing the RNA export system and RNA reporter transcripts with an editable barcode in the 3′ UTR. After 48 hours, supernatant was collected, RNA was extracted and reverse transcribed, PCR was used to amplify the ˜600 bp region encompassing the editable barcode, then Sanger sequencing was performed. Agarose gel electrophoresis confirmed the presence of expected ˜600 bp amplicon (data not shown). Sequencing reads confirmed readout of the editable barcode. Results demonstrate that exported RNA reporters containing barcodes can be sequenced and therefore used to conduct longitudinal analysis of population dynamics and lineage.

This example demonstrates that systems disclosed herein successfully export RNA reporters and can enable longitudinal single-cell analysis of population dynamics and lineage. It was shown that several embodiments provided herein based on various engineered RNA exporters successfully export RNA in a specific and quantitatively precise and reproducible manner (See e.g., FIG. 2). It was further shown that optimized RNA reporters can be controllably exported across a broad range of rates of export (See e.g., FIG. 2). Editable barcodes were recovered and sequenced within the exported reporter RNA from supernatant of mammalian cell cultures demonstrating the capability to longitudinally monitor population dynamics and lineage. Finally, it was shown that the engineered RNA exporters do not perturb normal cellular physiology and are non-toxic (See e.g., FIG. 6).

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A nucleic acid composition, comprising:
one or more first polynucleotide(s) encoding an RNA exporter protein and/or one or more second polynucleotide(s) each encoding one or more reporter RNA molecule(s),
wherein the RNA exporter protein comprises:
an RNA-binding domain,
a membrane-binding domain, and
an interaction domain capable of nucleating self-assembly, and
wherein a plurality of RNA exporter proteins are capable of self-assembling into lipid-enveloped nanoparticles (LNs) secreted from a reporter cell in which the RNA exporter proteins are expressed, thereby generating a population of LNs comprising exported reporter RNA molecule(s).

2. The nucleic acid composition of claim 1,
(A) wherein the reporter RNA molecule(s) each comprise packing signal(s), one or more cell barcode(s), and a reporter barcode; or
(B) wherein the nucleic acid composition further comprises one or more third polynucleotide(s) each encoding one or more packing RNA molecule(s), wherein the LNs further comprise exported packing RNA molecule(s), wherein the packing RNA molecule(s) comprise a capture domain, wherein the reporter RNA molecule(s) comprise a hybridization domain capable of hybridizing to the capture domain, and wherein:
(i) the reporter RNA molecule(s) each comprise a reporter barcode and one or more cell barcodes; and the packing RNA molecule(s) each comprise packing signal(s);
(ii) the reporter RNA molecule(s) each comprise a reporter barcode; and the packing RNA molecule(s) each comprise packing signal(s) and one or more cell barcode(s); and/or
(iii) the reporter RNA molecule(s) each comprise packing signal(s) and a reporter barcode; and the packing RNA molecule(s) each comprise one or more cell barcode(s).

3. The nucleic acid composition of claim 2, wherein the cell barcode(s) comprise:
a clone barcode, where each reporter cell of a population of reporter cells has a single clone barcode, wherein the sequence of clone barcode is unique to each reporter cell of the population of reporter cells at an initial time point, and wherein progeny cells arising from cell division of the same reporter cell constitute a clonal population wherein each clone comprises the same clone barcode;
a subpopulation barcode, wherein a population of reporter cells comprises one or more reporter cell subpopulations, where each reporter cell subpopulation has a single subpopulation barcode, wherein the sequence of the subpopulation barcode is unique to each reporter cell subpopulation of the population of reporter cells at an initial time point, and wherein progeny cells arising from cell division of the same reporter cell share the same subpopulation barcode; and/or
a lineage barcode, where each reporter cell of a population of reporter cells has a single lineage barcode, wherein the lineage barcode is not static, wherein the lineage barcode is an editable barcode, wherein at least about 10 percent of progeny cells arising from cell division of a reporter cell have a lineage barcode different than progeny cells arising from cell division of the same reporter cell.

4. The nucleic acid composition of claim 2, wherein the packing RNA molecule(s) and/or reporter RNA molecule(s) are mRNA, and wherein the reporter barcode, cell barcode(s) and/or packing signal(s) are situated in the 5′UTR and/or 3′UTR.

5. The nucleic acid composition of claim 3, comprising one or more fourth polynucleotide(s) encoding an editor and/or a targeting molecule, wherein the editor is a base editor capable of base editing the lineage barcode, and wherein said base editing comprises: adenine (A)-to-guanine (G) base editing and/or cytosine (C)- to-thymine (T) base editing.

6. The nucleic acid composition of claim 2, wherein the RNA binding domain is capable of binding the packing signal(s), and wherein the packing RNA molecule(s) and/or reporter RNA molecule(s) is specifically packaged into the LNs via interaction of the packing signal(s) with the RNA-binding domain of the RNA exporter protein.

7. The nucleic acid composition of claim 2, wherein the packing signal(s) comprise an array of at least about 2, 3, 4, 5, 6, 7, 8, 9, 10, 11, 12, 13, or 14, tandem repeats of an aptamer.

8. The nucleic acid composition of claim 2, wherein the RNA binding domain comprises or is derived from an RNA binding protein, and wherein:
the packing signal(s) comprise a Ku binding hairpin and the RNA binding protein is Ku;
the packing signal(s) comprise a telomerase Sm7 binding motif and the RNA binding protein is Sm7;
the packing signal(s) comprise an MS2 phage operator stem-loop and the RNA binding protein is MS2 Coat Protein (MCP),
the packing signal(s) comprise a PP7 phage operator stem-loop and the RNA binding protein is PP7 Coat Protein (PCP);
the packing signal(s) comprise an SfMu phage Com stem-loop and the RNA binding protein is Com RNA binding protein;
the packing signal(s) comprise a PUF binding site (PBS) and the RNA binding protein is Pumilio/fem-3 mRNA binding factor (PUF); and/or
the packing signal(s) comprise an MMLV packing signal (Psi) and the RNA binding protein is MMLV.

9. The nucleic acid composition of claim 1, wherein the RNA exporter protein comprises at least a portion of a viral capsid protein.

10. The nucleic acid composition of claim 1, wherein the RNA exporter protein comprises an amino acid sequence that is at least 80%, 85%, 90%, 95%, 98%, 99%, or 100% identical to any one of SEQ ID NOS: 1-24.

11. The nucleic acid composition of claim 1,
wherein the one or more reporter RNA molecule(s) comprise a plurality of reporter RNA molecules; and
wherein each of the reporter RNA molecules comprises a unique reporter barcode indicating a unique cell type and/or a unique cell state of the reporter cell from which it is derived.

12. The nucleic acid composition of claim 11, wherein the presence and/or amount of an exported reporter RNA molecule comprising a unique reporter barcode is correlated with the presence and/or amount of the unique cell type and/or a unique cell state in said reporter cell.

13. The nucleic acid composition of claim 11, wherein the degree of expression and/or degradation of the plurality of reporter RNA molecules is associated with the presence and/or amount of the unique cell type and/or a unique cell state.

14. The nucleic acid composition of claim 11, wherein a unique cell type and/or a unique cell state:
comprises a unique gene expression pattern; and/or
is caused by hereditable, environmental, and/or idiopathic factors.

15. The nucleic acid composition of claim 11, wherein the unique cell state and/or unique cell type is characterized by:
(i) aberrant signaling of one or more signal transducer(s);
(ii) one or more of cell proliferation, stress pathways, oxidative stress, stress kinase activation, DNA damage, lipid metabolism, carbohydrate regulation, metabolic activation including Phase I and Phase II reactions, Cytochrome P-450 induction or inhibition, ammonia detoxification, mitochondrial function, peroxisome proliferation, organelle function, cell cycle state, morphology, apoptosis, DNA damage, metabolism, signal transduction, cell differentiation, cell-cell interaction and cell to non-cellular compartment;
(iii) one or more of acute phase stress, cell adhesion, AH-response, anti-apoptosis and apoptosis, antimetabolism, anti-proliferation, arachidonic acid release, ATP depletion, cell cycle disruption, cell matrix disruption, cell migration, cell proliferation, cell regeneration, cell-cell communication, cholestasis, differentiation, DNA damage, DNA replication, early response genes, endoplasmic reticulum stress, estogenicity, fatty liver, fibrosis, general cell stress, glucose deprivation, growth arrest, heat shock, hepatotoxicity, hypercholesterolemia, hypoxia, immunotox, inflammation, invasion, ion transport, liver regeneration, cell migration, mitochondrial function, mitogenesis, multidrug resistance, nephrotoxicity, oxidative stress, peroxisome damage, recombination, ribotoxic stress, sclerosis, steatosis, teratogenesis, transformation, disrupted translation, transport, and tumor suppression; and/or
(iv) one or more of nutrient deprivation, hypoxia, oxidative stress, hyperproliferative signals, oncogenic stress, DNA damage, ribonucleotide depletion, replicative stress, and telomere attrition, promotion of cell cycle arrest, promotion of DNA-repair, promotion of apoptosis, promotion of genomic stability, promotion of senescence, and promotion of autophagy, regulation of cell metabolic reprogramming, regulation of tumor microenvironment signaling, inhibition of cell stemness, survival, and invasion.

16. The nucleic acid composition of claim 1, wherein the LNs contacted with RNase are capable of protecting reporter RNA molecule(s) comprised therein from RNase-mediated degradation.

17. The nucleic acid composition of claim 1, wherein:
the average diameter of the LNs of the population of LNs is about 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, 205 nm, 210 nm, 215 nm, 220 nm, 225 nm, 230 nm, 235 nm, 240 nm, 245 nm, 250 nm, 300 nm, 400 nm, or 500 nm; and/or
the LNs have a minimum diameter and/or a maximum diameter of about 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, 205 nm, 210 nm, 215 nm, 220 nm, 225 nm, 230 nm, 235 nm, 240 nm, 245 nm, 250 nm, 300 nm, 400 nm, or 500 nm.

18. The nucleic acid composition of claim 2, wherein the abundance of reporter RNA molecule(s) exported to the exterior of a reporter cell is at least about 2-fold higher as compared to a reporter cell wherein (i) the packing signal(s) are absent from the reporter RNA molecule(s) and/or packing RNA molecule(s) and/or (ii) the RNA exporter protein does not comprise an RNA binding domain.

19. A population of reporter cells comprising:
one or more first polynucleotide(s) encoding an RNA exporter protein and one or more second polynucleotide(s) each encoding one or more reporter RNA molecule(s),
wherein each of the reporter RNA molecules comprises a unique reporter barcode indicating a unique cell type and/or a unique cell state of the reporter cell from which it is derived,
wherein the RNA exporter protein comprises:
an RNA-binding domain,
a membrane-binding domain; and
an interaction domain capable of nucleating self-assembly, and
wherein a plurality of RNA exporter proteins are capable of self-assembling into lipid-enveloped nanoparticles (LNs) secreted from a reporter cell in which the RNA exporter proteins are expressed, thereby generating a population of LNs comprising exported reporter RNA molecule(s).

20. A method of determining the cell type and/or cell state of one or more reporter cells, comprising:
providing a population of reporter cells comprising: one or more first polynucleotide(s) encoding an RNA exporter protein and one or more second polynucleotide(s) each encoding one or more reporter RNA molecule(s),
wherein each of the reporter RNA molecules comprises a unique reporter barcode indicating a unique cell type and/or a unique cell state of the reporter cell from which it is derived,
wherein the RNA exporter protein comprises:
an RNA-binding domain,
a membrane-binding domain; and
an interaction domain capable of nucleating self-assembly, and
wherein a plurality of RNA exporter proteins are capable of self-assembling into lipid-enveloped nanoparticles (LNs) secreted from a reporter cell in which the RNA exporter proteins are expressed, thereby generating a population of LNs comprising exported reporter RNA molecule(s)
isolating a plurality of exported reporter RNA molecule(s) at one or more time points; and
obtaining sequence information of the plurality of exported reporter RNA molecule(s), or products thereof, to determine the cell type and/or cell state of the reporter cell(s).
